(19) United States
(12) Patent Application Publication
(10) Pub. No.: US 2004/0258663 A1
(43) Pub. Date: Dec. 23, 2004

(54) Compositions and methods for enhanced mucosal delivery of interferon alpha

(75) Inventors: Steven C. Quay, Edmonds, WA (US); Mohammed Abd El-Shafy, Hauppauge, NY (US)

Correspondence Address: Nastech Pharmaceutical Company Inc., 3450 Monte Villa Parkway, Bothell, WA, 98021-8906, US

(73) Assignee: Nastech Pharmaceutical Company Inc.

(21) Appl. No.: 10/840,536

(22) Filed: May 6, 2004

Related U.S. Application Data
(60) Provisional application No. 60/469,079, filed on May 8, 2003.

(51) Int. Cl. A61K 38/21

(52) U.S. Cl. 424/85.7

(57) ABSTRACT

Compositions and methods are provided for intranasal delivery of interferon-α yielding improved pharmacokinetic and pharmacodynamic results. In certain aspects of the invention, the interferon-α is delivered to the intranasal mucosa along with one or more intranasal delivery-enhancing agent(s) to yield substantially increased absorption and/or bioavailability of the interferon-α and/or a substantially decreased time to maximal concentration of interferon-α in a tissue of a subject as compared to controls where the interferon-α is administered to the same intranasal site alone or formulated according to previously disclosed reports. The enhancement of intranasal delivery of interferon-α according to the methods and compositions of the present invention allows for the effective pharmaceutical use of these agents to treat a variety of diseases and conditions in mammalian subjects.

### This claims the benefit under 35 U.S.C. §119 (e) of U. S. Provisional Application No. 60/469,079 filed on May 8, 2003, the entire contents of which are incorporated herein by reference

### BACKGROUND OF THE INVENTION

The teachings of all of the references cited herein are incorporated in their entirety by reference.

Infection with hepatitis C (HCV) virus causes an inflammation of the liver. It is the most common chronic blood-borne infection in the United States. According to the U.S. Centers for Disease Control and Prevention, approximately 1.8% of the U.S. population, or 3.9 million Americans, have been infected with the virus. About 35,000 new cases of hepatitis C infection are estimated to occur in the United States each year. The hepatitis C virus is blood-borne. Common routes of infection include needlestick accidents among healthcare workers; blood transfusions before mid-1992 (after 1992, blood banks began rigorous screening for the hepatitis C virus with effective new testing measures); and the use of recreational injection drugs (e.g., sharing needles). However, there are other modes of transmission and factors that may also put people at risk for contracting hepatitis C.

Interferon α (IFN-α), for example, interferon α-2b, IntronA® (interferon α-2b; Schering Corporation), interferon α-2a, Interferon alfacon, and PEG-Intron™ (PEG covalent conjugate to interferon-α-2b; Schering Corporation) is useful for treatment of hepatitis C infection. A unmet need exists in the art to increase safety and efficacy of IFN-α therapy for viral infection. The wide antiviral range of IFN-α therapy results from modulation of multiple biochemical pathways that have different antiviral effects and act on different parts of the various viral replication cycles. IFN-α induces an array of potent proteins regulating viral and cellular growth. In addition, IFN-α activates key components of the cellular immune system important in viral recognition. Plasma levels of IFN-α are increased in AIDS patients and in other viral infections. IFN-α is one treatment of choice for patients with chronic or acute hepatitis B and C infections. IFN-α is approved for the treatment of condyloma acuminata (genital or venereal warts caused by papilloma virus infection). IFN-α has also been used to treat papillomavirus warts of the larynx and skin (common warts). IFN-α has also been used to prevent and treat rhinovirus infection (common cold). IFN-α in combination with ribavarin has been used to treat hepatitis C infection. IFN-α in combination with zidovudine (azidothymidine) has been tested in patients to treat early HIV infection. Methods and formulations for IFN-α delivery, optimally at sustained levels that correspond substantially to normal physiological patterns of IFN-α secretion and action are needed in order to optimize dosing schedules without causing intolerable side effects. Treatment with IFN-α by intranasal administration by methods and compositions of the prior art has demonstrated an unfavorable side-effect profile. Repeated intranasal administration with compositions of the prior art progressively damages the nasal mucosa, so that long-term prophylaxis is not possible. Long term prophylaxis has resulted in bleeding of the nasal mucosa and poor absorption by the nasal mucosa.

Treatment of HIV infected individuals with IFN-α in combination with GM-CSF and zidovudine reduces HIV viral load but results in toxic side effects. Toxic side effects include IFN-α dose dependent increase in neutropenia, lymphokine-like side effects, anorexia and weight loss, fatigue, and anemia. An unmet need exists in the art to increase safety and efficacy of IFN-α therapy for anti-tumor treatment. Interferons appear to express potent antitumor effects both directly and indirectly—directly by exerting antiproliferative effects on target tumor cells by a cytostatic mechanism that slows the growth of tumor cells; by increasing the length of tumor cell multiplication cycle; by induction of tumor cell differentiation; or by induction of tumor cell apoptosis. Indirect effects include inhibition of angiogenesis, enhancement of the immune response, and activation of host cytotoxic effector cells to more efficiently lyse target tumor cells. Some human tumors respond well to interferon therapy. Beneficial clinical therapeutic activity of IFN-α as a single agent has been demonstrated in hairy cell leukemia, chronic myelogenous leukemia (CML), B and T cell lymphoma, midgut carcinoid tumors, metastasizing renal cell carcinoma, Kaposi's sarcoma, malignant melanoma, follicular lymphoma, and myeloma.

Thus there is a need to provide methods and formulations for enhanced delivery, optimally at sustained levels, of interferon-α via intranasal delivery, and action to optimize dosing schedules without causing intolerable side effects.

### DESCRIPTION OF THE INVENTION

The present invention fulfills the foregoing needs and satisfies additional objects and advantages by providing novel, effective methods and compositions for intranasal delivery of interferon-α yielding improved pharmacokinetic and pharmacodynamic results. In certain aspects of the invention, the interferon-α is delivered to the intranasal mucosa along with one or more intranasal delivery-enhancing agent(s) to yield substantially increased absorption and/or bioavailability of the interferon-α and/or a substantially decreased time to maximal concentration of interferon-α in a tissue of a subject as compared to controls where the interferon-α is administered to the same intranasal site alone or formulated according to previously disclosed reports.

The enhancement of intranasal delivery of interferon-α according to the methods and compositions of the present invention allows for the effective pharmaceutical use of these agents to treat a variety of diseases and conditions in mammalian subjects.

The methods and compositions provided herein provide for enhanced delivery of interferon-α across nasal mucosal barriers to reach novel target sites for drug action in an enhanced, therapeutically effective rate or concentration of delivery. In certain aspects, employment of one or more intranasal delivery-enhancing agents facilitates the effective delivery of interferon-α to a targeted, extracellular or cellular compartment, for example the systemic circulation, a selected cell population, tissue or organ. Exemplary targets for enhanced delivery in this context are target physiological compartments, tissues, organs and fluids (e.g., within the blood serum, liver, central nervous system (CNS) or cerebral spinal fluid (CSF)) or selected tissues or cells of the liver, bone, muscle, cartilage, pituitary, hypothalamus, kidney, lung, heart, testes, skin, or peripheral nervous system.

The enhanced delivery methods and compositions of the present invention provide for therapeutically effective mucosal delivery of interferon-α for prevention or treatment of a variety of disease and conditions in mammalian subjects. The interferon-α can be administered via a variety of mucosal routes, for example by contacting interferon-α to a nasal mucosal epithelium, a bronchial or pulmonary mucosal epithelium, an oral, gastric, intestinal or rectal mucosal epithelium, or a vaginal mucosal epithelium. Typically, the methods and compositions are directed to or formulated for intranasal delivery (e.g., nasal mucosal delivery or intranasal mucosal delivery).

In one aspect of the invention, pharmaceutical formulations suitable for intranasal administration are provided that comprise a therapeutically effective amount of interferon-α and one or more intranasal delivery-enhancing agents as described herein, which formulations are effective in a nasal mucosal delivery method of the invention to prevent the onset or progression of disease related to viral infection or tumor in a mammalian subject, or to alleviate one or more clinically well-recognized symptoms of viral infection or cancer, e.g., a solid tumor, in a mammalian subject.

In another aspect of the invention, pharmaceutical formulations suitable for intranasal administration are provided that comprise a therapeutically effective amount of interferon-α and one or more intranasal delivery-enhancing agents as described herein, which formulation is effective in a nasal mucosal delivery method of the invention to alleviate symptoms or prevent the onset or lower the incidence or severity of, for example, chronic or acute hepatitis B and hepatitis C infection, condyloma acuminata (genital or venereal warts caused by papilloma virus infection), papillomavirus warts of the larynx and skin (common warts), rhinovirus infection (common cold), early HIV infection treated with IFN-α in combination with zidovudine (azidothymidine). Pharmaceutical formulations and methods of the present invention invention are effective to alleviate symptoms or prevent the onset or lower the incidence or severity of, for example, hairy cell leukemia, AIDS-related Kaposi's sarcoma, chronic myelogenous leukemia (CML), B and T cell lymphoma, midgut carcinoid tumors, metastasizing renal cell carcinoma, malignant melanoma, follicular lymphoma, and myeloma. Within these and related methods, the IFN-α may be administered alone or in combination with IFN-β or other immune modifiers such as steroids or glatiramer acetate injection.

In more detailed aspects of the invention, methods and compositions for intranasal delivery of interferon-α incorporate one or more intranasal delivery enhancing agent(s) combined in a pharmaceutical formulation together with, or administered in a coordinate nasal mucosal delivery protocol with, a therapeutically effective amount of IFN-α. These methods and compositions provide enhanced nasal transmucosal delivery of the interferon-α, often in a pulsatile delivery mode to maintain continued release of interferon-α to yield more consistent (normalized) or elevated therapeutic levels of interferon-α in the blood serum, central nervous system (CNS), cerebral spinal fluid (CSF), or in another selected physiological compartment or target tissue or organ for treatment of disease. Normalized and elevated therapeutic levels of interferon-α are determined, for example, by an increase in bioavailability (e.g., as measured by maximal concentration (Cₘₐₓ) or the area under concentration vs. time curve (AUC) for an intranasal effective amount of interferon-α) and/or an increase in delivery rate (e.g., as measured by time to maximal concentration (tₘₐₓ), Cₘₐₓ, and or AUC). Normalized and elevated high therapeutic levels of interferon-α in the blood serum, central nervous system (CNS), or cerebral spinal fluid (CSF) may be achieved in part by repeated intranasal administration to a subject within a selected dosage period, for example an 8, 12, or 24 hour dosage period.

In an alternative embodiment, normalized and elevated therapeutic levels of interferon-α are determined, for example, by an increase in bioavailability and/or an increase in delivery rate as measured in the central nervous system (CNS) or cerebral spinal fluid (CSF), (e.g., as measured by tₘₐₓ, Cₘₐₓ, or AUC for an intranasal effective amount of interferon-α in the CNS or CSF).

To maintain more consistent or normalized therapeutic levels of interferon-α, the pharmaceutical formulations of the present invention are often repeatedly administered to the nasal mucosa of the subject, for example, one, two or more times within a 24 hour period, four or more times within a 24 hour period, six or more times within a 24 hour period, or eight or more times within a 24 hour period. The methods and compositions of the present invention yield improved pulsatile delivery to maintain normalized and/or elevated therapeutic levels of interferon- α, e.g., in the blood serum. The methods and compositions of the invention enhance transnasal mucosal delivery of interferon-α to a selected target tissue or compartment by at least a two- to five- fold increase, more typically a five- to ten-fold increase, and commonly a ten- to twenty-five- up to a fifty-fold increase (e.g., as measured by tₘₐₓCₘₐₓ, and/or AUC, in the hepatic portal vein, blood serum, or in another selected physiological compartment or target tissue or organ for delivery), compared to the efficacy of delivery of interferon-α administered alone or using a previously-described delivery method, for example a previously-described mucosal delivery, intramuscular delivery, subcutaneous delivery, intravenous delivery, and/or parenteral delivery method.

In more detailed aspects of the invention, the methods and compositions of the present invention provide improved and/or sustained delivery of interferon-α to the blood serum, lymphatic system, CNS, and/or CSF. In one exemplary embodiment, an intranasal effective amount of interferon-α and one or more intranasal delivery enhancing agent(s) is contacted with a nasal mucosal surface of a subject to yield enhanced mucosal delivery of interferon-α to the central nervous system (CNS) or cerebral spinal fluid (CSF) of the subject, for example to effectively treat chronic or acute hepatitis C virus (HCV) infection in cases where HCV replication has been demonstrated in the CNS. In certain embodiments, the methods and compositions of the invention provide improved and sustained delivery of interferon-α to hepatic and extrahepatic sites of HCV infection, including the CNS and CSF, and will effectively treat one or more symptoms of HCV infection, including in cases where conventional interferon-α therapy yields poor results or unacceptable adverse side effects.

Often the formulations of the invention are administered to a nasal mucosal surface of the subject. In certain embodiments, the interferon-α is a human interferon-α-2b, (IntronA®, Schering Corporation) or a pharmaceutically acceptable salt or derivative thereof. A mucosally effective dose within the pharmaceutical formulations of the present invention comprises, for example, between about 2 million IU and 36 million IU interferon α-2b recombinant (or between about 8 ng and 140 ng interferon α-2b recombinant). In certain embodiments, an effective dose of the pharmaceufical formulation comprising interferon-α is, for example, 3 million IU, 6 million IU, or 12 million IU of interferon α-2b recombinant (or about 12 ng, 24 ng or 48 ng of interferon α-2b recombinant). The pharmaceutical formulations of the present invention may be administered one or more times daily, or 3 times per week or weekly for between one week and 96 weeks. In certain embodiments, the pharmaceutical formulations of the invention is administered two times daily, four times daily, six times daily, or eight times daily. In related embodiments, the mucosal (e.g., intranasal) formulations comprising interferon-α(s) and one or more delivery-enhancing agent(s) administered via a repeated dosing regimen yields an area under the concentration curve (AUC) for interferon-α in the blood plasma or CSF following repeated dosing that is about 25% or greater compared to an area under the concentration curve (AUC) for interferon-α in the plasma or CSF following one or more subcutaneous injections of the same or comparable amount of interferon-α. In other embodiments, the mucosal formulations of the invention administered via a repeated dosing regimen yields an area under the concentration curve (AUC) for interferon-α in the blood plasma or CSF following repeated dosing that is about 40%, 80%, 100%, 150%, or greater compared to an area under the concentration curve (AUC) for interferon-α in the plasma or CSF, following one or more subcutaneous injections of the same or comparable amount of interferon-α.

In certain detailed aspects of the invention, a stable pharmaceutical formulation is provided which comprises interferon-α and one or more intranasal delivery-enhancing agent(s), wherein the formulation administered intranasally to a mammalian subject yields a peak concentration of interferon-α in the blood plasma (Cₘₐₓ) following intranasal administration to the subject by methods and compositions of the present invention is about 25% or greater compared to a peak concentration of interferon-α in the plasma following subcutaneous injection to the mammalian subject. Within related methods, the formulation is administered to a nasal mucosal surface of the subject.

In other detailed embodiments of the invention, the intranasal formulation of the interferon-α(s) and one or more delivery-enhancing agent(s) yields a peak concentration of interferon-α in the blood plasma (Cₘₐₓ) following intranasal administration to the subject that is about 40% or greater compared to a peak concentration of interferon-αin the plasma following subcutaneous injection of a comparable dose of interferon-α to the subject. Alternately, the intranasal formulation of the present invention may yield a peak concentration of interferon-α in the blood plasma (Cₘₐₓ) that is about 80%, 100% or 150%, or greater compared to the peak concentration of interferon-α in the plasma following subcutaneous injection to the mammalian subject.

The methods and compositions of the invention will often serve to improve interferon-α dosing schedules and thereby maintain normalized and/or elevated, therapeutic levels of interferon-α in the subject. In certain embodiments, the invention provides compositions and methods for intranasal delivery of interferon-α, wherein interferon-α dosage normalized and sustained by repeated, typically pulsatile, delivery to maintain more consistent, and in some cases elevated, therapeutic levels. In exemplary embodiments, the time to maximum concentration (tₘₐₓ) of interferon-α in the blood serum will be from about 0.1 to 4.0 hours, alternatively from about 0.4 to 1.5 hours, and in other embodiments from about 0.7 to 1.5 hours or from about 1.0 to 1.3 hours. Thus, repeated intranasal dosing with the formulations of the invention, on a schedule ranging from about 0.1 to 2.0 hours between doses, will maintain normalized, sustained therapeutic levels of interferon-α to maximize clinical benefits while minimizing the risks of excessive exposure and side effects.

Within other detailed embodiments of the invention, the foregoing methods and formulations are administered to a mammalian subject to yield enhanced blood plasma levels, CNS, CSF or other tissue levels of the interferon-α by administering a formulation comprising an intranasal effective amount of interferon-α and one or more intranasal delivery-enhancing agents and one or more sustained release-enhancing agents. The sustained release-enhancing agents, for example, may comprise a polymeric delivery vehicle. In exemplary embodiments, the sustained release-enhancing agent may comprise polyethylene glycol (PEG) coformulated or coordinately delivered with interferon-α and one or more intranasal delivery-enhancing agents. PEG may be covalently bound to interferon-α. The sustained release-enhancing methods and formulations of the present invention will increase residence time (RT) of the interferon-α at a site of administration and will maintain a basal level of the interferon-α over an extended period of time in blood plasma, CNS, CSF, or other tissue in the mammalian subject.

Within other detailed embodiments of the invention, the foregoing methods and formulations are administered to a mammalian subject to yield enhanced blood plasma levels, CNS, CSF or other tissue levels of the interferon-α to maintain basal levels of interferon-α over an extended period of time. Exemplary methods and formulations involve administering a pharmaceutical formulation comprising an intranasal effective amount of interferon-α and one or more intranasal delivery-enhancing agents to a mucosal surface of the subject, in combination with intramuscular or subcutaneous administration of a second pharmaceutical formulation comprising interferon-α. Maintenance of basal levels of interferon-α is particularly useful for treatment and prevention of disease, for example, chronic renal failure, acute myocardial infarction, congestive heart failure, and autoimmune disease.

The foregoing mucosal drug delivery formulations and preparative and delivery methods of the invention provide improved mucosal delivery of interferon-α to mammalian subjects. These compositions and methods can involve combinatorial formulation or coordinate administration of one or more interferon-α(s) with one or more mucosal (e.g., intranasal) delivery-enhancing agents. Among the mucosal delivery-enhancing agents to be selected from to achieve these formulations and methods are (a) aggregation inhibitory agents; (b) charge modifying agents; (c) pH control agents; (d) degradative enzyme inhibitors; (e) mucolytic or mucus clearing agents; (f) ciliostatic agents; (g) membrane penetration-enhancing agents (e.g., (i) a surfactant, (ii) a bile salt, (ii) a phospholipid or fatty acid additive, mixed micelle, liposome, or carrier, (iii) an alcohol, (iv) an enamine, (v) an NO donor compound, (vi) a long-chain amphipathic molecule (vii) a small hydrophobic penetration enhancer; (viii) sodium or a salicylic acid derivative; (ix) a glycerol ester of acetoacetic acid (x) a clyclodextrin or beta-cyclodextrin derivative, (xi) a medium-chain fatty acid, (xii) a chelating agent, (xiii) an amino acid or salt thereof, (xiv) an N-acetylamino acid or salt thereof, (xv) an enzyme degradative to a selected membrane component, (ix) an inhibitor of fatty acid synthesis, (x) an inhibitor of cholesterol synthesis; or (xi) any combination of the membrane penetration enhancing agents of (i)-(x)); (h) modulatory agents of epithelial junction physiology, such as nitric oxide (NO) stimulators, chitosan, and chitosan derivatives; (i) vasodilator agents; (j) selective transport-enhancing agents; and (k) stabilizing delivery vehicles, carriers, supports or complex-forming species with which the interferon-α(s) is/are effectively combined, associated, contained, encapsulated or bound to stabilize the active agent for enhanced nasal mucosal delivery.

In various embodiments of the invention, interferon-α is combined with one, two, three, four or more of the mucosal (e.g., intranasal) delivery-enhancing agents recited in (a)-(k), above. These mucosal delivery-enhancing agents may be admixed, alone or together, with the interferon-α, or otherwise combined therewith in a pharmaceutically acceptable formulation or delivery vehicle. Formulation of interferon-α with one or more of the mucosal delivery-enhancing agents according to the teachings herein (optionally including any combination of two or more mucosal delivery-enhancing agents selected from (a)-(k) above) provides for increased bioavailability of the interferon-α following delivery thereof to a mucosal (e.g., nasal mucosal) surface of a mammalian subject.

Intranasal delivery-enhancing agents are employed which enhance delivery of interferon-α into or across a nasal mucosal surface. For passively absorbed drugs, the relative contribution of paracellular and transcellular pathways to drug transport depends upon the pKa, partition coefficient, molecular radius and charge of the drug, the pH of the luminal environment in which the drug is delivered, and the area of the absorbing surface. The intranasal delivery-enhancing agent of the present invention may be a pH control agent. The pH of the pharmaceutical formulation of the present invention is a factor affecting absorption of interferon-α via paracellular and transcellular pathways to drug transport. In one embodiment, the pharmaceutical formulation of the present invention is pH adjusted to between about pH 3.0 to 8.0. In a further embodiment, the pharmaceutical formulation of the present invention is pH adjusted to between about pH 3.5 to 7.5. In a further embodiment, the pharmaceutical formulation of the present invention is pH adjusted to between about pH 4.0 to 5.0. In a further embodiment, the pharmaceutical formulation of the present invention is pH adjusted to between about pH 4.0 to 4.5.

In still other embodiments of the invention, pharmaceutical compositions and methods are provided wherein one or more of the interferon-α compounds or formulations described herein are administered coordinately or in a combinatorial formulation with one or more steroid or corticosteroid compound(s). These compositions in some embodiments are effective following mucosal administration to alleviate one or more symptom(s) of inflammation, nasal irritation, rhinitis, or allergy without unacceptable adverse side effects. In other embodiments, these combinatorial formulations or coordinate administration methods are effective to alleviate one or more symptom(s) of a viral infection or tumor.

Other combinatorial formulations for use within the invention comprise a stable pharmaceutical composition comprising an effective amount of one or more cytokine(s) or growth factor(s) formulated for mucosal delivery to a mammalian subject in combination with one or more steroid or corticosteroid compound(s), wherein the formulation is effective following mucosal administration to alleviate one or more symptom(s) of inflammation, nasal irritation, rhinitis, or allergy, or one or more symptom(s) of a viral infection or tumor without unacceptable adverse side effects. The combinatorial formulations of a cytokine and steroid may or may not contain mucosal delivery-enhancing agent(s) as described herein.

In more detailed embodiments, the combinatorial formulations and coordinate administration methods involving a cytokine or growth factor and steroid employ one or more steroid or corticosteroid compound(s) selected from triamcinolone, methylprednisolone, prednisolone, prednisone, fluticasone, betamethasone, dexamethasone, hydrocortisone, cortisone, flunisolide, beclomethasone dipropionate, budesonide, amcinonide, clobetasol, clobetasone, desoximetasone, diflorasone, diflucortolone, fluocinolone, fluocinonide, flurandrenolide, fluticasone, halcinonide, halobetasol, hydrocortisone butyrate, hydrocortisone valerate, and mometasone.

Nasal mucosal delivery of interferon-α according to the methods and compositions of the invention will often yield effective delivery and bioavailability that approximates dosing achieved by continuous administration methods. In other aspects, the invention provides enhanced nasal mucosal delivery that permits the use of a lower dosage and significantly reduces the incidence of interferon-α-related side effects. Because continuous infusion of interferon-α outside the hospital setting is otherwise impractical, mucosal delivery of interferon-α as provided herein yields unexpected advantages that allow sustained delivery of interferon-α, with the accrued benefits, for example, of improved patient-to-patient dose variability.

As noted above, the present invention provides improved methods and compositions for nasal mucosal delivery of interferon-α to mammalian subjects for treatment or prevention of a variety of diseases and conditions. Examples of appropriate mammalian subjects for treatment and prophylaxis according to the methods of the invention include, but are not restricted to, humans and non-human primates, livestock species, such as horses, cattle, sheep, and goats, and research and domestic species, including dogs, cats, mice, rats, guinea pigs, and rabbits.

In order to provide better understanding of the present invention, the following definitions are provided:

Interferon-α:

As used herein, “interferon-α” or “IFN-α” refers to interferon-α in native-sequence or in variant form, and from any source, whether natural, synthetic, or recombinant. Examples include human interferon-α (h IFN-α), which is natural or recombinant IFN-α with the human native sequence (also known as: leukocyte interferon, Type I interferon, B-cell interferon, buffy coat interferon, foreign cell induced interferon, lymphoblast interferon, lymphoblastoid interferon, mamalwa interferon, pH2-stable interferon, or RSV-induced factor). Recombinant interferon-α (r IFN-α), which refers to any IFN-α or variant produced by means of recombinant DNA technology. One group of therapeutic compounds of interest for mucosal delivery is interferon α (IFN-α), for example, human interferon α-2b, (IntronA®, Schering Corporation).

As used herein, at least 23 different subtypes of IFN-α are known. The individual proteins have molecular masses between 16-27 kDa and consist of proteins with lengths of 156-166 and 172 amino acids. All IFN-α subtypes possess a common conserved sequence region between amino acid positions 115-151 while the amino-terminal ends are variable. Many IFN-α subtypes differ in their sequences at only one or two positions. Naturally occurring variants also include proteins truncated by 10 amino acids at the carboxy-terminal end. Disulfide bonds are formed between cysteines at positions 1/98 and 29/138. The disulfide bond 29/138 is essential for biological activity while the 1/98 bond can be reduces without affecting biological activity. All IFN-α forms contain a potential glycosylation site but most subtypes are not glycosylated. In contrast to IFN-gamma, IFN-α proteins are stable at pH2.

There are at least 13 different IFN-α genes. They have a length of 1-2 kb and are clustered on human chromosome 9q(p23-13). The 13 IFN-α genes have 80 to 95% homology with one another at the nucleotide level. In some cell systems expression of some subtypes (IFN-α-1, IFN-α-2, IFN-α-4 ) is stronger than those of others. IFN-α genes do not contain intron sequences found in many other eukaryotic genes. Based upon the structures two types of IFN-α genes, designated class I and II, are distinguished. They encode proteins of 156-166 and 172 amino acids, respectively. Deletions covering 9p22 are observed frequently in cells of lymphoblastoid leukemias. Biron, Immunity, 14: 661-664, 2001, incorporated herein by reference.

Additional disclosures teach detailed methods and tools pointing to specific structural and functional characteristics that define effective therapeutic uses of IFN-α, and further disclose a diverse, additional array of these agents that are useful within the invention. IFN-α isoforms are produced by monocytes/macrophages, lymphoblastoid cells, fibroblasts, and a number of different cell types following induction by viruses, nucleic acids, glucocorticoid hormones, and low-molecular weight substances (n-butyrate, 5-bromodeoxy uridine). The growth of some tumor cell types in vitro is inhibited by IFN-α which may also stimulate the synthesis of tumor-associated cell surface antigens. In renal carcinomas IFN-α reduces the expression of EGF receptors. IFN-α also inhibits the growth of fibroblasts and monocytes in vitro. IFN-α also inhibits the proliferation of B-cell in vitro and blocks the synthesis of antibodies. IFN-α also selectively blocks the expression of some mitochondrial genes. IFN-α specifically induces the expression of a number of genes (for example, Mx protein). These genes contain regulatory DNA sequences within their promoter regions (ISRE ; Interferon-stimulated response element) that function as binding sites for a number of transcription factors. Some of these genes are also expressed in response to other interferons.

IFN-α inhibits the expression of a number of cytokines in hematopoietic progenitor cells that in turn induce a state of competence in these cells allowing them to pass from the G0 into the S-phase of the cell cycle.

The occurrence of spontaneous antibodies directed against IFN-α has been observed in patients with certain types of autoimmune diseases, generalized virus infections, and a number of tumors. Some inbred strains of mice appear to produce constitutively antibodies directed against IFN-α or IFN-β.

IFN-α shows a number of biological activities. All known subtypes of IFN-α show antiviral, antiparasitic, antiproliferative activities in suitable bioassays although IFN-α subtypes may differ in relative activities.

Antiviral Activity

As noted above, the instant invention provides improved and useful methods and compositions for mucosal delivery of IFN-α to prevent and treat viral infection by human immunodeficiency virus (HIV), acute or chronic hepatitis B, acute or chronic hepatitis C, or papilloma virus in mammalian subjects. The wide antiviral range of IFN-α results from modulation of multiple biochemical pathways that have different antiviral effects and act on different parts of the various viral replication cycles. IFN-α induces an array of potent proteins regulating viral and cellular growth. In addition, IFN-α activates key components of the cellular immune system important in viral recognition. Plasma levels of IFN-α are increased in HIV-infected patients and in other viral infections. IFN-α is one treatment of choice for patients with chronic or acute hepatitis B or hepatitis C infections. IFN-α is approved for the treatment of condyloma acuminata (genital or venereal warts).

Antitumor Action

As noted above, the instant invention provides improved and useful methods and compositions for mucosal delivery of IFN-α to prevent and treat tumors in mammalian subjects. IFN-α appear to express potent antitumor effects both directly and indirectly—directly by exerting antiproliferative effects on target tumor cells by a cytostatic mechanism that slows the growth of tumor cells by increasing the length of their cell multiplication cycle, by induction of differentiation, and by induction of apoptosis. IFN-α acts indirectly by inhibition of angiogenesis, enhancement of the immune response, and by activating the host cytotoxic effector cells to more efficiently lyse target tumor cells.

Some human tumors respond well to interferon therapy. The instant invention provides improved and useful methods and compositions of the present invention for nasal mucosal delivery of IFN-α to prevent and treat hairy cell leukemia, chronic myelogenous leukemia (CML), B and T cell lymphoma, midgut carcinoid tumors, metastasizing renal cell carcinoma, Kaposi's sarcoma, malignant melanoma, follicular lymphoma, and myeloma in mammalian subjects. Beneficial clinical therapeutic activity of IFN-α as a single agent has been demonstrated in treatment of these tumors by useful methods and compositions of the instant invention.

Hairy cell leukemia constitutes approximately 2 percent of all leukemias. Treatment with improved and useful methods and compositions of the present invention for nasal mucosal delivery of IFN-α markedly improves blood and bone marrow parameters. The number of necessary blood transfusions is reduced and the frequency of life-threatening infections is also reduced.

Treatment of disseminated Kaposi sarcomas with improved and useful methods and compositions of the present invention for nasal mucosal delivery of IFN-α results in complete or partial remissions in approximately 30-40 percent of the patients. In patients with advanced malignant melanomas treatment with a combination of IFN-α and chemotherapy (Dacarbazin, DTIC) has been found to be particularly effective and to be superior to treatment with IFN-α alone. Complete remissions and also a significant increase in survival times have been observed in responders. Intralesional therapy with IFN-α has been found to cause almost complete disappearance of tumors in 80 percent of patients with basaliomas.

Improved and useful methods and compositions of the present invention for nasal mucosal delivery of IFN-α delivered at moderate and high doses are one of the most effective forms of treatment of metastasizing renal carcinomas. Response rates of combinations of vinblastin and IFN-α are approximately 25 percent higher than those with interferon alone. Response rates have been reported improved by combining IFN-α with antineoplastic agents or other cytokines. Combination therapy with systemically administered IFN-α and IL2 has resulted in long-term remissions in 30 percent of patients with metastatic renal cell carcinoma.

Treatment of chronic myelogenous leukemia (CML) with improved and useful methods and compositions of the present invention for nasal mucosal delivery of IFN-α causes hematological remissions in most patients and has been shown to cause a complete elimination of the PHI-(Philadelphia chromosome)-positive cells in the bone marrow of some patients. Corssmit, et al., J. Interferon Cytokine Res., 20(12): 1039-1047, 2000, incorporated herein by reference.

Immunomodulatory Action

As noted above, the instant invention provides improved and useful methods and compositions of the present invention for nasal mucosal delivery of IFN-α to prevent and treat an inflammatory response in mammalian subjects. IFN-α induces multiple alterations in the distribution and functional properties of leukocytes and exerts proinflammatory as well as anti-inflammatory effects within the cytokine network. IFN-α can modulate the ability of various immunologic effector cells to interact with malignant cells or with virus-infected cells, for example, through enhanced expression of class I major histocompatibility complex (MHC) antigen and cellular adhesion molecules. Corssmit, et al., J. Interferon Cytokine Res., 20(12): 1039-1047, 2000, incorporated herein by reference.

Methods and Compositions of Delivery

Improved methods and compositions for mucosal administration of interferon-α to mammalian subjects optimize interferon-α dosing schedules. The present invention provides mucosal delivery of interferon-α formulated with one or more mucosal delivery-enhancing agents wherein interferon-α dosage release is substantially normalized and/or sustained for an effective delivery period of interferon-α release ranges from approximately 0.1 to 2.0 hours; 0.4 to 1.5 hours; 0.7 to 1.5 hours; or 1.0 to 1.3 hours; following mucosal administration. The sustained release of interferon-α is achieved may be facilitated by repeated administration of exogenous interferon-α utilizing methods and compositions of the present invention.

Compositions and Methods of Sustained Release

Improved compositions and methods for mucosal administration of interferon-α to mammalian subjects optimize interferon-α dosing schedules. The present invention provides improved mucosal (e.g., nasal) delivery of a formulation comprising interferon-α in combination with one or more mucosal delivery-enhancing agents and an optional sustained release-enhancing agent or agents. Mucosal delivery-enhancing agents of the present invention yield an effective increase in delivery, e.g., an increase in the maximal plasma concentration (Cₘₐₓ) to enhance the therapeutic activity of mucosally-administered interferon-α . A second factor affecting therapeutic activity of interferon-α in the blood plasma and CNS is residence time (RT). Sustained release-enhancing agents, in combination with intranasal delivery-enhancing agents, increase Cₘₐₓ, and increase residence time (RT) interferon-α. Polymeric delivery vehicles and other agents and methods of the present invention that yield sustained release-enhancing formulations, for example, polyethylene glycol (PEG), are disclosed herein. The present invention provides an improved interferon-α a delivery method and dosage form for treatment of symptoms related viral infection or tumor disease in mammalian subjects.

Maintenance of Basal Levels of Interferon-α.

Improved compositions and methods for mucosal administration of interferon-α to mammalian subjects optimize interferon-α dosing schedules. The present invention provides improved nasal mucosal delivery of a formulation comprising interferon-α and intranasal delivery-enhancing agents in combination with intramuscular or subcutaneous administration of interferon-α. Formulations and methods of the present invention maintain relatively consistent basal levels of interferon-α, for example throughout a 2 to 24 hour, 4-16 hour, or 8-12 hour period following a single dose administration or attended by a multiple dosing regimen of 2-6 sequential administrations. Maintenance of basal levels of interferon-α is particularly useful for treatment and prevention of disease, for example, acute or chronic hepatitis B or hepatitis C, without unacceptable adverse side effects.

Within the mucosal delivery formulations and methods of the invention, the interferon-α is frequently combined or coordinately administered with a suitable carrier or vehicle for mucosal delivery. As used herein, the term “carrier” means a pharmaceutically acceptable solid or liquid filler, diluent or encapsulating material. A water-containing liquid carrier can contain pharmaceutically acceptable additives such as acidifying agents, alkalizing agents, antimicrobial preservatives, antioxidants, buffering agents, chelating agents, complexing agents, solubilizing agents, humectants, solvents, suspending and/or viscosity-increasing agents, tonicity agents, wetting agents or other biocompatible materials. A tabulation of ingredients listed by the above categories, can be found in the U.S. Pharmacopeia National Formulary, pp. 1857-1859, 1990, which is incorporated herein by reference. Some examples of the materials which can serve as pharmaceutically acceptable carriers are sugars, such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols, such as propylene glycol; polyols such as glycerin, sorbitol, mannitol and polyethylene glycol; esters such as ethyl oleate and ethyl laurate; agar; buffering agents such as magnesium hydroxide and aluminum hydroxide; alginic acid; pyrogen free water; isotonic saline; Ringer's solution, ethyl alcohol and phosphate buffer solutions, as well as other non toxic compatible substances used in pharmaceutical formulations. Wetting agents, emulsifiers and lubricants such as sodium lauryl sulfate and magnesium stearate, as well as coloring agents, release agents, coating agents, sweetening, flavoring and perfuming agents, preservatives and antioxidants can also be present in the compositions, according to the desires of the formulator. Examples of pharmaceutically acceptable antioxidants include water soluble antioxidants such as ascorbic acid, cysteine hydrochloride, sodium bisulfite, sodium metabisulfite, sodium sulfite and the like; oil-soluble antioxidants such as ascorbyl palmitate, butylated hydroxyanisole (BHA), butylated hydroxytoluene (BHT), lecithin, propyl gallate, alpha-tocopherol and the like; and metal-chelating agents such as citric acid, ethylenediamine tetraacetic acid (EDTA), sorbitol, tartaric acid, phosphoric acid and the like. The amount of active ingredient that can be combined with the carrier materials to produce a single dosage form will vary depending upon the particular mode of administration.

The mucosal formulations of the invention are generally sterile, particulate free and stable for pharmaceutical use. As used herein, the term “particulate free” means a formulation that meets the requirements of the USP specification for small volume parenteral solutions. The term “stable” means a formulation that fulfills all chemical and physical specifications with respect to identity, strength, quality, and purity which have been established according to the principles of Good Manufacturing Practice, as set forth by appropriate governmental regulatory bodies.

Within the mucosal delivery compositions and methods of the invention, various delivery-enhancing agents are employed which enhance delivery of interferon-α into or across a mucosal surface. In this regard, delivery of interferon-α across the mucosal epithelium can occur “transcellularly” or “paracellularly”. The extent to which these pathways contribute to the overall flux and bioavailability of the interferon-α depends upon the environment of the mucosa, the physico-chemical properties the active agent, and on the properties of the mucosal epithelium. Paracellular transport involves only passive diffusion, whereas transcellular transport can occur by passive, facilitated or active processes. Generally, hydrophilic, passively transported, polar solutes diffuse through the paracellular route, while more lipophilic solutes use the transcellular route. Absorption and bioavailability (e.g., as reflected by a permeability coefficient or physiological assay), for diverse, passively and actively absorbed solutes, can be readily evaluated, in terms of both paracellular and transcellular delivery components, for any selected interferon-α within the invention. These values can be determined and distinguished according to well known methods, such as in vitro epithelial cell culture permeability assays (see, e.g., Hilgers, et al., Pharm. Res.7:902-910, 1990; Wilson et al.,J. Controlled Release 11: 25-40, 1990; Artursson. I., Pharm. Sci. 79: 476-482, 1990; Cogburn et al., Pharm. Res. 8:210-216, 1991; Pade et al., Pharmaceutical Research 14: 1210-1215, 1997, each incorporated herein by reference).

For passively absorbed drugs, the relative contribution of paracellular and transcellular pathways to drug transport depends upon the pKa, partition coefficient, molecular radius and charge of the drug, the pH of the luminal environment in which the drug is delivered, and the area of the absorbing surface. The paracellular route represents a relatively small fraction of accessible surface area of the nasal mucosal epithelium. In general terms, it has been reported that cell membranes occupy a mucosal surface area that is a thousand times greater than the area occupied by the paracellular spaces. Thus, the smaller accessible area, and the size- and charge-based discrimination against macromolecular permeation would suggest that the paracellular route qould be a generally less favorable route than transcellular delivery for drug transport. Surprisingly, the methods and compositions of the invention provide for significantly enhanced transport of biotherapeutics into and across mucosal epithelia via the paracellular route. Therefore, the methods and compositions of the invention successfully target both paracellular and transcellular routes, alternatively or within a single method or composition.

As used herein, “mucosal delivery-enhancing agents” include agents which enhance the release or solubility (e.g., from a formulation delivery vehicle), diffusion rate, penetration capacity and timing, uptake, residence time, stability, effective half-life, peak or sustained concentration levels, clearance and other desired mucosal delivery characteristics (e.g., as measured at the site of delivery, or at a selected target site of activity such as the bloodstream or central nervous system) of interferon-α or other biologically active compound(s). Enhancement of mucosal delivery can thus occur by any of a variety of mechanisms, for example by increasing the diffusion, transport, persistence or stability of interferon-α, increasing membrane fluidity, modulating the availability or action of calcium and other ions that regulate intracellular or paracellular permeation, solubilizing mucosal membrane components (e.g., lipids), changing non-protein and protein sulfhydryl levels in mucosal tissues, increasing water flux across the mucosal surface, modulating epithelial junctional physiology, reducing the viscosity of mucus overlying the mucosal epithelium, reducing mucociliary clearance rates, and other mechanisms.

As used herein, an “mucosally effective amount of interferon-α” contemplates effective mucosal delivery of interferon-α to a target site for drug activity in the subject that may involve a variety of delivery or transfer routes. For example, a given active agent may find its way through clearances between cells of the mucosa and reach an adjacent vascular wall, while by another route the agent may, either passively or actively, be taken up into mucosal cells to act within the cells or be discharged or transported out of the cells to reach a secondary target site, such as the systemic circulation. The methods and compositions of the invention may promote the translocation of active agents along one or more such alternate routes, or may act directly on the mucosal tissue or proximal vascular tissue to promote absorption or penetration of the active agent(s). The promotion of absorption or penetration in this context is not limited to these mechanisms.

As used herein “peak concentration (Cₘₐₓ) of interferon-α in a blood plasma”, “area under concentration vs. time curve (AUC) of interferon-α in a blood plasma”, “time to maximal plasma concentration (tₘₐₓ) of interferon-α in a blood plasma” are pharmacokinetic parameters known to one skilled in the art. (Laursen et al., Eur. J. Endocrinology, 135: 309-315, 1996, incorporated herein by reference.) The “concentration vs. time curve” measures the concentration of interferon-α in a blood serum of a subject vs. time after administration of a dosage of interferon-α to the subject either by intranasal, intramuscular, subcutaneous, or other parenteral route of administration. “Cₘₐₓ” is the maximum concentration of interferon-α in the blood serum of a subject following a single dosage of interferon-α to the subject. “tₘₐₓ” is the time to reach maximum concentration of interferon-α in a blood serum of a subject following administration of a single dosage of interferon-α to the subject.

As used herein, “area under concentration vs. time curve (AUC) of interferon-α in a blood plasma” is calculated according to the linear trapezoidal rule and with addition of the residual areas. A decrease of 23% or an increase of 30% between two dosages would be detected with a probability of 90% (type II error β=10%). The “delivery rate” or “rate of absorption” is estimated by comparison of the time (tₘₐₓ) to reach the maximum concentration (Cₘₐₓ). Both Cₘₐₓ and tₘₐₓ are analyzed using non-parametric methods. Comparisons of the pharmacokinetics of intramuscular, subcutaneous, intravenous and intranasal interferon-α administrations were performed by analysis of variance (ANOVA). For pairwise comparisons a Bonferroni-Holmes sequential procedure was used to evaluate significance. The dose-response relationship between the three nasal doses was estimated by regression analysis. P<0.05 was considered significant. Results are given as mean values+/−SEM. (Laursen et al., 1996.)

As used herein, “pharmacokinetic markers” include any accepted biological marker that is detectable in an in vitro or in vivo system useful for modeling pharmacokinetics of mucosal delivery of one or more interferon-α compounds, or other biologically active agent(s) disclosed herein, wherein levels of the marker(s) detected at a desired target site following administration of the interferon-α compound(s) according to the methods and formulations herein, provide a reasonably correlative estimate of the level(s) of the interferon-α compound(s) delivered to the target site. Among many art-accepted markers in this context are substances induced at the target site by adminstration of the interferon-α compound(s) or orther biologically active agent(s).

Many known reagents that are reported to enhance mucosal absorption also cause irritation or damage to mucosal tissues (see, e.g., Swenson and Curatolo, Adv. Drug Delivery Rev. 8: 39-92, 1992, incorporated herein by reference). For example, in studies of intestinal absorption enhancing agents, the delivery-enhancing effects of various absorption-promoting agents are reportedly directly related to their membrane toxicity (see, e.g., Uchiyama et al., Biol. Pharm. Bull. 19: 1618-1621, 1996; Yamamoto et al., J. Pharm. Pharmacol. 48: 1285-1289, 1996, each incorporated herein by reference). In this regard, the combinatorial formulation and coordinate administration methods of the present invention incorporate effective, minimally toxic delivery-enhancing agents to enhance mucosal delivery of interferon-α and other biologically active macromolecules useful within the invention.

While the mechanism of absorption promotion may vary with different intranasal delivery-enhancing agents of the invention, useful reagents in this context will not substantially adversely affect the mucosal tissue and will be selected according to the physicochemical characteristics of the particular interferon-α or other active or delivery-enhancing agent. In this context, delivery enhancing agents that increase penetration or permeability of mucosal tissues will often result in some alteration of the protective permeability barrier of the mucosa. For such delivery-enhancing agents to be of value within the invention, it is generally desired that any significant changes in permeability of the mucosa be reversible within a time frame appropriate to the desired duration of drug delivery. Furthermore, there should be no substantial, cumulative toxicity, nor any permanent deleterious changes induced in the barrier properties of the mucosa with long-term use.

Within certain aspects of the invention, absorption-promoting agents for coordinate administration or combinatorial formulation with interferon-α of the invention are selected from small hydrophilic molecules, including but not limited to, dimethyl sulfoxide (DMSO), dimethylformamide, ethanol, propylene glycol, and the 2-pyrrolidones. Alternatively, long-chain amphipathic molecules, for example, deacylmethyl sulfoxide, azone, sodium laurylsulfate, oleic acid, and the bile salts, may be employed to enhance mucosal penetration of the interferon-α. In additional aspects, surfactants (e.g., polysorbates) are employed as adjunct compounds, processing agents, or formulation additives to enhance intranasal delivery of the interferon-α. These penetration enhancing agents typically interact at either the polar head groups or the hydrophilic tail regions of molecules which comprise the lipid bilayer of epithelial cells lining the nasal mucosa (Barry, Pharmacology of the Skin, Vol. 1, pp. 121-137, Shroot et al., Eds., Karger, Basel, 1987; and Barry, J. controlled Release 6: 85-97, 1987, each incorporated herein by reference). Interaction at these sites may have the effect of disrupting the packing of the lipid molecules, increasing the fluidity of the bilayer, and facilitating transport of the interferon-α across the mucosal barrier. Interaction of these penetration enhancers with the polar head groups may also cause or permit the hydrophilic regions of adjacent bilayers to take up more water and move apart, thus opening the paracellular pathway to transport of the interferon-α. In addition to these effects, certain enhancers may have direct effects on the bulk properties of the aqueous regions of the nasal mucosa. Agents such as DMSO, polyethylene glycol, and ethanol can, if present in sufficiently high concentrations in delivery environment (e.g., by pre-administration or incorporation in a therapeutic formulation), enter the aqueous phase of the mucosa and alter its solubilizing properties, thereby enhancing the partitioning of the interferon-α from the vehicle into the mucosa.

Additional mucosal delivery-enhancing agents that are useful within the coordinate administration and processing methods and combinatorial formulations of the invention include, but are not limited to, mixed micelles; enamines; nitric oxide donors (e.g., S-nitroso-N-acetyl-DL-penicillamine, NORI, NOR4—which are preferably co-administered with an NO scavenger such as carboxy-PITO or doclofenac sodium); sodium salicylate; glycerol esters of acetoacetic acid (e.g., glyceryl-1,3-diacetoacetate or 1,2-isopropylideneglycerine-3-acetoacetate); and other release-diffusion or intra- or trans-epithelial penetration-promoting agents that are physiologically compatible for mucosal delivery. Other absorption-promoting agents are selected from a variety of carriers, bases and excipients that enhance mucosal delivery, stability, activity or trans-epithelial penetration of the interferon-α. These include, inter alia, clyclodextrins and β-cyclodextrin derivatives (e.g., 2-hydroxypropyl-β-cyclodextrin and heptakis(2,6-di-O-methyl-β-cyclodextrin). These compounds, optionally conjugated with one or more of the active ingredients and further optionally formulated in an oleaginous base, enhance bioavailability in the mucosal formulations of the invention. Yet additional absorption-enhancing agents adapted for mucosal delivery include medium-chain fatty acids, including mono- and diglycerides (e.g., sodium caprate—extracts of coconut oil, Capmul), and triglycerides (e.g., amylodextrin, Estaram 299, Miglyol 810).

The mucosal therapeutic and prophylactic compositions of the present invention may be supplemented with any suitable penetration-promoting agent that facilitates absorption, diffusion, or penetration of interferon-α across mucosal barriers. The penetration promoter may be any promoter that is pharmaceutically acceptable. Thus, in more detailed aspects of the invention compositions are provided that incorporate one or more penetration-promoting agents selected from sodium salicylate and salicylic acid derivatives (acetyl salicylate, choline salicylate, salicylamide, etc.); amino acids and salts thereof (e.g. monoaminocarboxlic acids such as glycine, alanine, phenylalanine, proline, hydroxyproline, etc.; hydroxyamino acids such as serine; acidic amino acids such as aspartic acid, glutamic acid, etc; and basic amino acids such as lysine etc-inclusive of their alkali metal or alkaline earth metal salts); and N-acetylamino acids (N-acetylalanine, N-acetylphenylalanine, N-acetylserine, N-acetylglycine, N-acetyllysine, N-acetylglutamic acid, N-acetylproline, N-acetylhydroxyproline, etc.) and their salts (alkali metal salts and alkaline earth metal salts). Also provided as penetration-promoting agents within the methods and compositions of the invention are substances which are generally used as emulsifiers (e.g. sodium oleyl phosphate, sodium lauryl phosphate, sodium lauryl sulfate, sodium myristyl sulfate, polyoxyethylene alkyl ethers, polyoxyethylene alkyl esters, etc.), caproic acid, lactic acid, malic acid and citric acid and alkali metal salts thereof, pyrrolidonecarboxylic acids, alkylpyrrolidonecarboxylic acid esters, N-alkylpyrrolidones, proline acyl esters, and the like.

Within various aspects of the invention, improved nasal mucosal delivery formulations and methods are provided that allow delivery of interferon-α and other therapeutic agents within the invention across mucosal barriers between administration and selected target sites. Certain formulations are specifically adapted for a selected target cell, tissue or organ, or even a particular disease state. In other aspects, formulations and methods provide for efficient, selective endo- or transcytosis of interferon-α specifically routed along a defined intracellular or intercellular pathway. Typically, the interferon-α is efficiently loaded at effective concentration levels in a carrier or other delivery vehicle, and is delivered and maintained in a stabilized form, e.g., at the nasal mucosa and/or during passage through intracellular compartments and membranes to a remote target site for drug action (e.g., the blood stream or a defined tissue, organ, or extracellular compartment). The interferon-α may be provided in a delivery vehicle or otherwise modified (e.g., in the form of a prodrug), wherein release or activation of the interferon-α is triggered by a physiological stimulus (e.g. pH change, lysosomal enzymes, etc.) Often, the interferon-α is pharmacologically inactive until it reaches its target site for activity. In most cases, the interferon-α and other formulation components are non-toxic and non-immunogenic. In this context, carriers and other formulation components are generally selected for their abilitity to be rapidly degraded and excreted under physiological conditions. At the same time, formulations are chemically and physically stable in dosage form for effective storage.

Charge Modifying and pH Control Agents and Methods

Consistent with these general teachings, mucosal delivery of charged macromolecular species, including interferon-α and other biologically active agents, within the methods and compositions of the invention is substantially improved when the active agent is delivered to the mucosal surface in a substantially un-ionized, or neutral, electrical charge state.

Mucolytic and Mucus-Clearing Agents and Methods

Effective delivery of biotherapeutic agents via intranasal administration must take into account the decreased drug transport rate across the protective mucus lining of the nasal mucosa, in addition to drug loss due to binding to glycoproteins of the mucus layer. Normal mucus is a viscoelastic, gel-like substance consisting of water, electrolytes, mucins, macromolecules, and sloughed epithelial cells. It serves primarily as a cytoprotective and lubricative covering for the underlying mucosal tissues. Randomly distributed secretory cells located in the nasal epithelium and in other mucosal epithelia secrete mucus. The structural unit of mucus is mucin. This glycoprotein is mainly responsible for the viscoelastic nature of mucus, although other macromolecules may also contribute to this property. In airway mucus, such macromolecules include locally produced secretory IgA, IgM, IgE, lysozyme, and bronchotransferrin, which also play an important role in host defense mechanisms.

The thickness of mucus varies from organ to organ and between species. However, mucin glycoproteins obtained from different sources have similar overall amino acid and protein/carbohydrate compositions, although the molecular weight may vary over a wide. Mucin consists of a large protein core with oligosaccharide side-chains attached through the O-glycosidic linkage of galactose or N-acetyl glucosamine to hydroxyl groups of serine and threonine residues. Either sialic acid or L-fucose forms the terminal group of the side chain oligosaccharides with sialic acid (negatively charged at pH greater than 2.8) forming 50 to 60% of the terminal groups. The presence of cysteine in the end regions of the mucin core facilitates cross-linking of mucin molecules via disulfide bridge formation.

The coordinate administration methods of the instant invention optionally incorporate effective mucolytic or mucus-clearing agents, which serve to degrade, thin or clear mucus from intranasal mucosal surfaces to facilitate absorption of intranasally administered biotherapeutic agents. Within these methods, a mucolytic or mucus-clearing agent is coordinately administered as an adjunct compound to enhance intranasal delivery of the biologically active agent. Alternatively, an effective amount of a mucolytic or mucus-clearing agent is incorporated as a processing agent within a multi-processing method of the invention, or as an additive within a combinatorial formulation of the invention, to provide an improved formulation that enhances intranasal delivery of biotherapeutic compounds by reducing the barrier effects of intranasal mucus.

A variety of mucolytic or mucus-clearing agents are available for incorporation within the methods and compositions of the invention. Based on their mechanisms of action, mucolytic and mucus clearing agents can often be classified into the following groups: proteases (e.g., pronase, papain) that cleave the protein core of mucin glycoproteins; sulfhydryl compounds that split mucoprotein disulfide linkages; and detergents (e.g., Triton X-100, Tween 20) that break non-covalent bonds within the mucus. Additional compounds in this context include, but are not limited to, bile salts and surfactants, for example, sodium deoxycholate, sodium taurodeoxycholate, sodium glycocholate, and lysophosphatidylcholine.

The effectiveness of bile salts in causing structural breakdown of mucus is in the order deoxycholate>taurocholate>glycocholate. Other effective agents that reduce mucus viscosity or adhesion to enhance intranasal delivery according to the methods of the invention include, e.g., short-chain fatty acids, and mucolytic agents that work by chelation, such as N-acylcollagen peptides, bile acids, and saponins (the latter function in part by chelating Ca²⁺ and/or Mg²⁺ which play an important role in maintaining mucus layer structure).

Additional mucolytic agents for use within the methods and compositions of the invention include N-acetyl-L-cysteine (ACS), a potent mucolytic agent that reduces both the viscosity and adherence of bronchopulmonary mucus and is reported to modestly increase nasal bioavailability of human interferon-α in anesthetized rats (from 7.5 to 12.2%). These and other mucolytic or mucus-clearing agents are contacted with the nasal mucosa, typically in a concentration range of about 0.2 to 20 mM, coordinately with administration of the biologically active agent, to reduce the polar viscosity and/or elasticity of intranasal mucus.

Still other mucolytic or mucus-clearing agents may be selected from a range of glycosidase enzymes, which are able to cleave glycosidic bonds within the mucus glycoprotein. α-amylase and β-amylase are representative of this class of enzymes, although their mucolytic effect may be limited (Leiberman, J., Am. Rev. Respir. Dis. 97: 662, 1967, incorporated herein by reference). In contrast, bacterial glycosidases that allow these microorganisms to permeate mucus layers of their hosts are highly mucolytic active.

For selecting mucolytic agents for use within the methods and compositions of the invention, it is important to consider the chemical nature of both the mucolytic (or mucus-clearing) and biologically active agents. For example, the proteolytic enzyme pronase exhibits a very strong mucolytic activity at pH 5.0, as well as at pH 7.2. In contrast, the protease papain exhibited substantial mucolytic activity at pH 5.0, but no detectable mucolytic activity at pH 7.2. The reason for these differences in activity are explained in part by the distinct pH-optimum for papain, reported to be pH 5. Thus, mucolytic and other enzymes for use within the invention are typically delivered in formulations having a pH at or near the pH optimum of the subject enzyme.

For combinatorial use with most biologically active agents within the invention, including peptide and protein therapeutics, non-ionogenic detergents are generally also useful as mucolytic or mucus-clearing agents. These agents typically will not modify or substantially impair the activity of therapeutic polypeptides.

Ciliostatic Agents and Methods

Because the self-cleaning capacity of certain mucosal tissues (e.g., nasal mucosal tissues) by mucociliary clearance is necessary as a protective function (e.g., to remove dust, allergens, and bacteria), it has been generally considered that this function should not be substantially impaired by mucosal medications. Mucociliary transport in the respiratory tract is a particularly important defense mechanism against infections (Wasserman.,J. Allergy Clin. Immunol. 73: 17-19, 1984). To achieve this function, ciliary beating in the nasal and airway passages moves a layer of mucus along the mucosa to removing inhaled particles and microorganisms. During chronic bronchitis and chronic sinusitis, tracheal and nasal mucociliary clearance are often impaired (Wanner., Am. Rev. Respir. Dis. 116: 73-125, 1977, incorporated herein by reference). This is presumably due to either excess secretion (Dulfano, et al., Am. Rev. Respir. Dis. 104: 88-98, 1971), increased viscosity of mucus (Chen, et al.,J. Lab. Clin. Med. 91: 423-431, 1978, incorporated herein by reference), alterations in ciliary activity caused by decreased beat frequency loss of portions of the ciliated epithelium or to a combination of these factors. Decreased clearance presumably favors bacterial colonization of respiratory mucosal surfaces, predisposing the subject to infection. The ability to interfere with this host defense system may contribute significantly to a pathological organism's virulence.

Various reports show that mucociliary clearance can be impaired by mucosally administered drugs, as well as by a wide range of formulation additives including penetration enhancers and preservatives. For example, ethanol at concentrations greater than 2% has been shown to reduce the in vitro ciliary beating frequency. This may be mediated in part by an increase in membrane permeability that indirectly enhances flux of calcium ion, which, at high concentration, is ciliostatic, or by a direct effect on the ciliary axoneme or actuation of regulatory proteins involved in a ciliary arrest response. Exemplary preservatives (methyl-p-hydroxybenzoate (0.02% and 0.15%), propyl-p-hydroxybenzoate (0.02%), and chlorobutanol (0.5%)) reversibly inhibit ciliary activity in a frog palate model. Other common additives (EDTA (0.1%), benzalkoniuin chloride (0.01%), chlorhexidine (0.01%), phenylinercuric nitrate (0.002%), and phenylmercuric borate (0.002%), have been reported to inhibit mucociliary transport irreversibly. In addition, several penetration enhancers including STDHF, laureth-9, deoxycholate, deoxycholic acid, taurocholic acid, and glycocholic acid have been reported to inhibit ciliary activity in model systems.

Despite the potential for adverse effects on mucociliary clearance attributed to ciliostatic factors, ciliostatic agents nonetheless find use within the methods and compositions of the invention to increase the residence time of mucosally (e.g., intranasally) administered interferon-α and other biologically active agents disclosed herein. In particular, the delivery these agents within the methods and compositions of the invention is significantly enhanced in certain aspects by the coordinate administration or combinatorial formulation of one or more ciliostatic agents that function to reversibly inhibit ciliary activity of mucosal cells, to provide for a temporary, reversible increase in the residence time of the mucosally administered active agent(s). For use within these aspects of the invention, the foregoing ciliostatic factors, either specific or indirect in their activity, are all candidates for successful employment as ciliostatic agents in appropriate amounts (depending on concentration, duration and mode of delivery) such that they yield a transient (i.e., reversible) reduction or cessation of mucociliary clearance at a mucosal site of administration to enhance delivery of interferon-a and other biologically active agents disclosed herein, without unacceptable adverse side effects.

Within more detailed aspects, a specific ciliostatic factor is employed in a combined formulation or coordinate administration protocol with interferon-α and/or other biologically active agents disclosed herein. Various bacterial ciliostatic factors isolated and characterized in the literature may be employed within these embodiments of the invention. For example, Hingley, et al. (Infection and Immunity. 51: 254-262, 1986, have recently identified ciliostatic factors from the bacterium Pseudomonas aeruginosa. These are heat-stable factors released by Pseudomonas aeruginosa in culture supernatants that have been shown to inhibit ciliary function in epithelial cell cultures. Exemplary among these cilioinhibitory components are a phenazine derivative, a pyo compound (2-alkyl-4-hydroxyquinolines), and a rhamnolipid (also known as a hemolysin). Inhibitory concentrations of these and other active components were established by quantitative measures of ciliary motility and beat frequency. The pyo compound produced ciliostasis at concentrations of 50 μg/ml and without obvious ultrastructural lesions. The phenazine derivative also inhibited ciliary motility but caused some membrane disruption, although at substantially greater concentrations of 400 μg/ml. Limited exposure of tracheal explants to the rhamnolipid resulted in ciliostasis, which was associated with altered ciliary membranes. More extensive exposure to rhamnolipid was associated with removal of dynein arms from axonemes. It is proposed that these and other bacterial ciliostatic factors have evolved to enable P. aeruginosa to more easily and successfully colonize the respiratory tract of mammalian hosts. On this basis, respiratory bacteria are useful pathogens for identification of suitable, specific ciliostatic factors for use within the methods and compositions of the invention.

Several methods are available to measure mucociliary clearance for evaluating the effects and uses of ciliostatic agents within the methods and compositions of the invention. Nasal mucociliary clearance can be measured by monitoring the disappearance of visible tracers such as India ink, edicol orange powder, and edicol supra orange. These tracers are followed either by direct observation or with the aid of posterior rhinoscopy or a binocular operating microscope. This method simply measures the time taken by a tracer to travel a definite distance. In more modem techniques, radiolabeled tracers are administered as an aerosol and traced by suitably collimated detectors. Alternatively, particles with a strong taste like saccharin can be placed in the nasal passage and assayed to determine the time before the subject first perceives the taste is used as an indicator of mucociliary clearance.

Additional assays are known in the art for measuring ciliary beat activity. For example, a laser light scattering technique to measure tracheobronchial mucociliary activity is based on mono-chromaticity, coherence, and directionality of laser light. Ciliary motion is measured as intensity fluctuations due to the interference of Doppler-shifted scattered light. The scattered light from moving cilia is detected by a photomultiplier tube and its frequency content analyzed by a signal correlator yielding an autocorrelation function of the detected photocurrents. In this way, both the frequency and synchrony of beating cilia can be measured continuously. Through fiberoptic rhinoscopy, this method also allows the measurement of ciliary activity in the peripheral parts of the nasal passages.

In vitro assays for evaluating ciliostatic activity of formulations within the invention are also available. For example, a commonly used and accepted assay in this context is a rabbit tracheal explant system (Gabridge et al., Pediatr. Res. 1: 31-35, 1979; Chandler et al., Infect. Immun. 29: 1111-1116, 1980,). Other assay systems measure the ciliary beat frequency of a single cell or a small number of cells (Kennedy et al., Exp. Cell Res. 135: 147-156, 1981; Rutland et al., Lancet ii 564-565, 1980; Verdugo, et al., Pediatr. Res. 13: 131-135, 1979,).

Surface Active Agents and Methods

Within more detailed aspects of the invention, one or more membrane penetration-enhancing agents may be employed within a mucosal delivery method or formulation of the invention to enhance mucosal delivery of interferon-α and other biologically active agents disclosed herein. Membrane penetration enhancing agents in this context can be selected from: (i) a surfactant, (ii) a bile salt, (ii) a phospholipid additive, mixed micelle, liposome, or carrier, (iii) an alcohol, (iv) an enamine, (v) an NO donor compound, (vi) a long-chain amphipathic molecule (vii) a small hydrophobic penetration enhancer; (viii) sodium or a salicylic acid derivative; (ix) a glycerol ester of acetoacetic acid (x) a clyclodextrin or beta-cyclodextrin derivative, (xi) a medium-chain fatty acid, (xii) a chelating agent, (xiii) an amino acid or salt thereof, (xiv) an N-acetylamino acid or salt thereof, (xv) an enzyme degradative to a selected membrane component, (ix) an inhibitor of fatty acid synthesis, or (x) an inhibitor of cholesterol synthesis; or (xi) any combination of the membrane penetration enhancing agents recited in (i)-(x)

Certain surface-active agents are readily incorporated within the mucosal delivery formulations and methods of the invention as mucosal absorption enhancing agents. These agents, which may be coordinately administered or combinatorially formulated with interferon-α and other biologically active agents disclosed herein, may be selected from a broad assemblage of known surfactants. Surfactants, which generally fall into three classes: (1) nonionic polyoxyethylene ethers; (2) bile salts such as sodium glycocholate (SGC) and deoxycholate (DOC); and (3) derivatives of fusidic acid such as sodium taurodihydrofusidate (STDHF). The mechanisms of action of these various classes of surface active agents typically include solubilization of the biologically active agent. For proteins and peptides which often form aggregates, the surface active properties of these absorption promoters can allow interactions with proteins such that smaller units such as surfactant coated monomers may be more readily maintained in solution. These monomers are presumably more transportable units than aggregates. A second potential mechanism is the protection of the peptide or protein from proteolytic degradation by proteases in the mucosal environment. Both bile salts and some fusidic acid derivatives reportedly inhibit proteolytic degradation of proteins by nasal homogenates at concentrations less than or equivalent to those required to enhance protein absorption. This protease inhibition may be especially important for peptides with short biological half-lives.

Degradation Enzymes and Inhibitors of Fatty Acid and Cholesterol Synthesis

In related aspects of the invention, interferon-α and other biologically active agents for mucosal administration are formulated or coordinately administered with a penetration enhancing agent selected from a degradation enzyme, or a metabolic stimulatory agent or inhibitor of synthesis of fatty acids, sterols or other selected epithelial barrier components (see, e.g., U.S. Pat. No. 6,190,894). In one embodiment, known enzymes that act on mucosal tissue components to enhance permeability are incorporated in a combinatorial formulation or coordinate administration method of instant invention, as processing agents within the multi-processing methods of the invention. For example, degradative enzymes such as phospholipase, hyaluronidase, neuraminidase, and chondroitinase may be employed to enhance mucosal penetration of interferon-α and other biologically active agents (see, e.g., Squier Brit. J. Dermatol. 111: 253-264, 1984; Aungst and Rogers Int. J. Pharm. 53: 227-235, 1989,), without causing irreversible damage to the mucosal barrier. In one embodiment, chondroitinase is employed within a method or composition as provided herein to alter glycoprotein or glycolipid constituents of the permeability barrier of the mucosa, thereby enhancing mucosal absorption interferon-α and other biologically active agents disclosed herein.

With regard to inhibitors of synthesis of mucosal barrier constituents, it is noted that free fatty acids account for 20-25% of epithelial lipids by weight. Two rate limiting enzymes in the biosynthesis of free fatty acids are acetyl CoA carboxylase and fatty acid synthetase. Through a series of steps, free fatty acids are metabolized into phospholipids. Thus, inhibitors of free fatty acid synthesis and metabolism for use within the methods and compositions of the invention include, but are not limited to, inhibitors of acetyl CoA carboxylase such as 5-tetradecyloxy-2-furancarboxylic acid (TOFA); inhibitors of fatty acid synthetase; inhibitors of phospholipase A such as gomisin A, 2-(p-amylcinnamyl)amino-4-chlorobenzoic acid, bromophenacyl bromide, monoalide, 7,7-dimethyl-5,8-eicosadienoic acid, nicergoline, cepharanthine, nicardipine, quercetin, dibutyryl-cyclic AMP, R-24571, N-oleoylethanolamine, N-(7-nitro-2,1,3-benzoxadiazol-4-yl) phosphostidyl serine, cyclosporine A, topical anesthetics, including dibucaine, prenylamine, retinoids, such as all-trans and 13-cis-retinoic acid, W-7, trifluoperazine, R-24571 (calmidazolium), 1-hexadocyl-3-trifluoroethyl glycero-sn-2-phosphomenthol (MJ33); calcium channel blockers including nicardipine, verapamil, diltiazem, nifedipine, and nimodipine; antimalarials including quinacrine, mepacrine, chloroquine and hydroxychloroquine; beta blockers including propanalol and labetalol; calmodulin antagonists; EGTA; thimersol; glucocorticosteroids including dexamethasone and prednisolone; and nonsteroidal anti-inflammatory agents including indomethacin and naproxen.

Free sterols, primarily cholesterol, account for 20-25% of the epithelial lipids by weight. The rate limiting enzyme in the biosynthesis of cholesterol is 3-hydroxy-3-methylglutaryl (HMG) CoA reductase. Inhibitors of cholesterol synthesis for use within the methods and compositions of the invention include, but are not limited to, competitive inhibitors of (HMG) CoA reductase, such as simvastatin, lovastatin, fluindostatin (fluvastatin), pravastatin, mevastatin, as well as other HMG CoA reductase inhibitors, such as cholesterol oleate, cholesterol sulfate and phosphate, and oxygenated sterols, such as 25-OH— and 26-OH— cholesterol; inhibitors of squalene synthetase; inhibitors of squalene epoxidase; inhibitors of DELTA7 or DELTA24 reductases such as 22,25-diazacholesterol, 20,25-diazacholestenol, AY9944, and triparanol.

Each of the inhibitors of fatty acid synthesis or the sterol synthesis inhibitors may be coordinately administered or combinatorially formulated with one or more interferon-α compound(s) and other biologically active agents disclosed herein to achieve enhanced epithelial penetration of the active agent(s). An effective concentration range for the sterol inhibitor in a therapeutic or adjunct formulation for mucosal delivery is generally from about 0.0001% to about 20% by weight of the total, more typically from about 0.01% to about 5%.

Nitric Oxide Donor Agents and Methods

Within other related aspects of the invention, a nitric oxide (NO) donor is selected as a membrane penetration-enhancing agent to enhance mucosal delivery of interferon-α and other biologically active agents disclosed herein. Recently, Salzman et al. (Am. J. Physiol. 268: G361-G373, 1995, incorporated herein by reference) reported that NO donors increase the permeability of water-soluble compounds across Caco-2 cell monolayers with neither loss of cell viability nor lactate dehydrogenase (LDH) release. In addition, Utoguchi et al. (Pharm. Res. 15: 870-876, 1998, incorporated herein by reference) demonstrated that the rectal absorption of insulin was remarkably enhanced in the presence of NO donors, with attendant low cytotoxicity as evaluated by the cell detachment and LDH release studies in Caco-2 cells.

Various NO donors are known in the art and are useful in effective concentrations within the methods and formulations of the invention. Exemplary NO donors include, but are not limited to, nitroglycerine, nitropruside, NOC5 [3-(2-hydroxy-1-(methyl-ethyl)-2-nitrosohydrazino)- 1-propanamine], NOC 12 [N-ethyl-2-(1-ethyl-hydroxy-2-nitrosohydrazino)-ethanamine], SNAP [S-nitroso-N-acetyl-DL-penicillamine], NORI and NOR4. Efficacy of these and other NO donors, as well as other mucosal delivery-enhancing agents disclosed herein, for enhancing mucosal delivery of interferon-α and other biologically active agents can be evaluated routinely according to known efficacy and cytotoxicity assay methods (e.g., involving control coadministration of an NO scavenger, such as carboxy-PIIO) as described by Utoguchi et al., Pharm. Res. 15: 870-876, 1998 (incorporated herein by reference).

Within the methods and compositions of the invention, an effective amount of a selected NO donor is coordinately administered or combinatorially formulated with interferon-α and/or other biologically active agents disclosed herein, into or through the mucosal epithelium.

Vasodilator Agents and Methods

Yet another class of absorption-promoting agents that shows beneficial utility within the coordinate administration and combinatorial formulation methods and compositions of the invention are vasoactive compounds, more specifically vasodilators. These compounds function within the invention to modulate the structure and physiology of the submucosal vasculature, increasing the transport rate of interferon-α and other biologically active agents into or through the mucosal epithelium and/or to specific target tissues or compartments (e.g., the systemic circulation or central nervous system.).

Vasodilator agents for use within the invention typically cause submucosal blood vessel relaxation by either a decrease in cytoplasmic calcium, an increase in nitric oxide (NO) or by inhibiting myosin light chain kinase. They are generally divided into 9 classes: calcium antagonists, potassium channel openers, ACE inhibitors, angiotensin-II receptor antagonists, α-adrenergic and imidazole receptor antagonists, β1 -adrenergic agonists, phosphodiesterase inhibitors, eicosanoids and NO donors.

Despite chemical differences, the pharmacokinetic properties of calcium antagonists are similar. Absorption into the systemic circulation is high, and these agents therefore undergo considerable first-pass metabolism by the liver, resulting in individual variation in pharmacokinetics. Except for the newer drugs of the dihydropyridine type (amlodipine, felodipine, isradipine, nilvadipine, nisoldipine and nitrendipine), the half-life of calcium antagonists is short. Therefore, to maintain an effective drug concentration for many of these may require delivery by multiple dosing, or controlled release formulations, as described elsewhere herein. Treatment with the potassium channel opener minoxidil may also be limited in manner and level of administration due to potential adverse side effects.

ACE inhibitors prevent conversion of angiotensin-I to angiotensin-II, and are most effective when renin production is increased. Since ACE is identical to kininase-II, which inactivates the potent endogenous vasodilator bradykinin, ACE inhibition causes a reduction in bradykinin degradation. ACE inhibitors provide the added advantage of cardioprotective and cardioreparative effects, by preventing and reversing cardiac fibrosis and ventricular hypertrophy in animal models. The predominant elimination pathway of most ACE inhibitors is via renal excretion. Therefore, renal impairment is associated with reduced elimination and a dosage reduction of 25 to 50% is recommended in patients with moderate to severe renal impairment.

With regard to NO donors, these compounds are particularly useful within the invention for their additional effects on mucosal permeability. In addition to the above-noted NO donors, complexes of NO with nucleophiles called NO/nucleophiles, or NONOates, spontaneously and nonenzymatically release NO when dissolved in aqueous solution at physiologic pH. In contrast, nitro vasodilators such as nitroglycerin require specific enzyme activity for NO release. NONOates release NO with a defined stoichiometry and at predictable rates ranging from <3 minutes for diethylamine/NO to approximately 20 hours for diethylenetriamine/NO (DETANO).

Within certain methods and compositions of the invention, a selected vasodilator agent is coordinately administered (e.g., systemically or intranasally, simultaneously or in combinatorially effective temporal association) or combinatorially formulated with interferon-α and other biologically active agent(s) in an amount effective to enhance the mucosal absorption of the active agent(s) to reach a target tissue or compartment in the subject (e.g., the systemic circulation or CNS).

Selective Transport-Enhancing Agents and Methods

Within certain aspects of the invention, methods and agents that target selective transport mechanisms and promote endo- or transcytocis of macromolecular drugs enhance mucosal delivery of biologically active agents. In this regard, the compositions and delivery methods of the invention optionally incorporate a selective transport-enhancing agent that facilitates transport of one or more biologically active agents. These transport-enhancing agents may be employed in a combinatorial formulation or coordinate administration protocol with interferon-α disclosed herein, to coordinately enhance delivery of one or more additional biologically active agent(s) across mucosal transport barriers, to enhance mucosal delivery of the active agent(s) to reach a target tissue or compartment in the subject (e.g., the mucosal epithelium, the systemic circulation or the CNS). Alternatively, the transport-enhancing agents may be employed in a combinatorial formulation or coordinate administration protocol to directly enhance mucosal delivery of interferon-α with or without enhanced delivery of an additional biologically active agent.

Exemplary selective transport-enhancing agents for use within this aspect of the invention include, but are not limited to, glycosides, sugar-containing molecules, and binding agents such as lectin binding agents, which are known to interact specifically with epithelial transport barrier components. For example, specific “bioadhesive” ligands, including various plant and bacterial lectins, which bind to cell surface sugar moieties by receptor-mediated interactions can be employed as carriers or conjugated transport mediators for enhancing mucosal, e.g., nasal delivery of biologically active agents within the invention. Certain bioadhesive ligands for use within the invention will mediate transmission of biological signals to epithelial target cells that trigger selective uptake of the adhesive ligand by specialized cellular transport processes (endocytosis or transcytosis). These transport mediators can therefore be employed as a “carrier system” to stimulate or direct selective uptake of interferon-α and other biologically active agent(s) into and/or through mucosal epithelia. These and other selective transport-enhancing agents significantly enhance mucosal delivery of macromolecular biopharmaceuticals (particularly peptides, proteins, oligonucleotides and polynucleotide vectors) within the invention. To utilize these transport-enhancing agents, general carrier formulation and/or conjugation methods as described elsewhere herein are used to coordinately administer a selective transport enhancer (e.g., a receptor-specific ligand) and a biologically active agent to a mucosal surface, whereby the transport-enhancing agent is effective to trigger or mediate enhanced endo- or transcytosis of the active agent into or across the mucosal epithelium and/or to additional target cell(s), tissue(s) or compartment(s).

Lectins are plant proteins that bind to specific sugars found on the surface of glycoproteins and glycolipids of eukaryotic cells. Concentrated solutions of lectins have a ‘mucotractive’ effect, and various studies have demonstrated rapid receptor mediated endocytocis (RME) of lectins and lectin conjugates (e.g., concanavalin A conjugated with colloidal gold particles) across mucosal surfaces. Additional studies have reported that the uptake mechanisms for lectins can be utilized for intestinal drug targeting in vivo. In certain of these studies, polystyrene nanoparticles (500 nm) were covalently coupled to tomato lectin and reported yielded improved systemic uptake after oral administration to rats.

Polymeric Delivery Vehicles and Methods

Within certain aspects of the invention, interferon-α and other biologically active agents disclosed herein, and delivery-enhancing agents as described above, are, individually or combinatorially, incorporated within a mucosally (e.g., nasally) administered formulation that includes a biocompatible polymer functioning as a carrier or base. Such polymer carriers include polymeric powders, matrices or microparticulate delivery vehicles, among other polymer forms. The polymer can be of plant, animal, or synthetic origin. Often the polymer is crosslinked. Additionally, in these delivery systems the biologically active agent (e.g., interferon-α), can be functionalized in a manner where it can be covalently bound to the polymer and rendered inseparable from the polymer by simple washing. In other embodiments, the polymer is chemically modified with an inhibitor of enzymes or other agents that can degrade or inactivate the biologically active agent(s) and/or delivery enhancing agent(s). In certain formulations, the polymer is a partially or completely water insoluble but water swellable polymer, e.g., a hydrogel. Polymers useful in this aspect of the invention are desirably water interactive and/or hydrophilic in nature to absorb significant quantities of water, and they often form hydrogels when placed in contact with water or aqueous media for a period of time sufficient to reach equilibrium with water. In more detailed embodiments, the polymer is a hydrogel which, when placed in contact with excess water, absorbs at least two times its weight of water at equilibrium when exposed to water at room temperature (see, e.g., U.S. Pat. No. 6,004,583,).

Drug delivery systems based on biodegradable polymers are preferred in many biomedical applications because such systems are broken down either by hydrolysis or by enzymatic reaction into non-toxic molecules. Manipulating the composition of the biodegradable polymer matrix controls the rate of degradation. These types of systems can therefore be employed in certain settings for long-term release of biologically active agents. Biodegradable polymers such as poly(glycolic acid) (PGA), poly-(lactic acid) (PLA), and poly(D,L-lactic-co-glycolic acid) (PLGA), have received considerable attention as possible drug delivery carriers, since the degradation products of these polymers have been found to have low toxicity. During the normal metabolic function of the body these polymers degrade into carbon dioxide and water (Mehta et al, J. Control. Rel. 29: 375-384, 1994). These polymers have also exhibited excellent biocompatibility.

For prolonging the biological activity of interferon-α and other biologically active agents disclosed herein, as well as optional delivery-enhancing agents, these agents may be incorporated into polymeric matrices, e.g., polyorthoesters, polyanhydrides, or polyesters. This yields sustained activity and release of the active agent(s), e.g., as determined by the degradation of the polymer matrix (Heller, Formulation and Delivery of Proteins and Peptides, pp. 292-305, Cleland et al., Eds., ACS Symposium Series 567, Washington DC, 1994; Tabata et al., Pharm. Res.10: 487-496, 1993; and Cohen et al.,Pharm. Res. 8: 713-720, 1991,). Although the encapsulation of biotherapeutic molecules inside synthetic polymers may stabilize them during storage and delivery, the largest obstacle of polymer-based release technology is the activity loss of the therapeutic molecules during the formulation processes that often involve heat, sonication or organic solvents (Tabata et al., Pharm. Res. 10: 487-496, 1993; and Jones et al., Drug Targeting and Delivery Series, New Delivery Systems for Recombinant Proteins—Practical Issues from Proof of Concept to Clinic, Vol. 4, pp. 57-67, Lee et al., Eds., Harwood Academic Publishers, 1995).

Absorption-promoting polymers contemplated for use within the invention may include derivatives and chemically or physically modified versions of the foregoing types of polymers, in addition to other naturally occurring or synthetic polymers, gums, resins, and other agents, as well as blends of these materials with each other or other polymers, so long as the alterations, modifications or blending do not adversely affect the desired properties, such as water absorption, hydrogel formation, and/or chemical stability for useful application. In more detailed aspects of the invention, polymers such as nylon, acrylan and other normally hydrophobic synthetic polymers may be sufficiently modified by reaction to become water swellable and/or form stable gels in aqueous media.

Suitable polymers for use within the invention should generally be stable alone and in combination with the selected biologically active agent(s) and additional components of a mucosal formulation, and form stable hydrogels in a range of pH conditions from about pH 1 to pH 10. More typically, they should be stable and form polymers under pH conditions ranging from about 3 to 9, without additional protective coatings. However, desired stability properties may be adapted to physiological parameters characteristic of the targeted site of delivery (e.g., nasal mucosa or secondary site of delivery such as the systemic circulation). Therefore, in certain formulations higher or lower stabilities at a particular pH and in a selected chemical or biological environment will be more desirable.

Absorption-promoting polymers of the invention may include polymers from the group of homo- and copolymers based on various combinations of the following vinyl monomers: acrylic and methacrylic acids, acrylamide, methacrylamide, hydroxyethylacrylate or methacrylate, vinylpyrrolidones, as well as polyvinylalcohol and its co- and terpolymers, polyvinylacetate, its co- and terpolymers with the above listed monomers and 2-acrylamido-2-methyl-propanesulfonic acid (AMPS®). Very useful are copolymers of the above listed monomers with copolymerizable functional monomers such as acryl or methacryl amide acrylate or methacrylate esters where the ester groups are derived from straight or branched chain alkyl, aryl having up to four aromatic rings which may contain alkyl substituents of 1 to 6 carbons; steroidal, sulfates, phosphates or cationic monomers such as N,N-dimethylaminoalkyl(meth)acrylamide, dimethylaminoalkyl(meth)acrylate, (meth)acryloxyalkyltrimethylammonium chloride, (meth)acryloxyalkyldimethylbenzyl ammonium chloride.

Additional absorption-promoting polymers for use within the invention are those classified as dextrans, dextrins, and from the class of materials classified as natural gums and resins, or from the class of natural polymers such as processed collagen, chitin, chitosan, pullalan, zooglan, alginates and modified alginates such as “Kelcoloid” (a polypropylene glycol modified alginate) gellan gums such as “Kelocogel”, Xanathan gums such as “Keltrol”, estastin, alpha hydroxy butyrate and its copolymers, hyaluronic acid and its derivatives, polylactic and glycolic acids.

A very useful class of polymers applicable within the instant invention are olefinically-unsaturated carboxylic acids containing at least one activated carbon-to-carbon olefinic double bond, and at least one carboxyl group; that is, an acid or functional group readily converted to an acid containing an olefinic double bond which readily functions in polymerization because of its presence in the monomer molecule, either in the alpha-beta position with respect to a carboxyl group, or as part of a terminal methylene grouping. Olefinically-unsaturated acids of this class include such materials as the acrylic acids typified by the acrylic acid itself, alpha-cyano acrylic acid, beta methylacrylic acid (crotonic acid), alpha-phenyl acrylic acid, beta-acryloxy propionic acid, cinnamic acid, p-chloro cinnamic acid, 1-carboxy-4-phenyl butadiene-1,3, itaconic acid, citraconic acid, mesaconic acid, glutaconic acid, aconitic acid, maleic acid, fumaric acid, and tricarboxy ethylene. As used herein, the term “carboxylic acid” includes the polycarboxylic acids and those acid anhydrides, such as maleic anhydride, wherein the anhydride group is formed by the elimination of one molecule of water from two carboxyl groups located on the same carboxylic acid molecule.

Representative acrylates useful as absorption-promoting agents within the invention include methyl acrylate, ethyl acrylate, propyl acrylate, isopropyl acrylate, butyl acrylate, isobutyl acrylate, methyl methacrylate, methyl ethacrylate, ethyl methacrylate, octyl acrylate, heptyl acrylate, octyl methacrylate, isopropyl methacrylate, 2-ethylhexyl methacrylate, nonyl acrylate, hexyl acrylate, n-hexyl methacrylate, and the like. Higher alkyl acrylic esters are decyl acrylate, isodecyl methacrylate, lauryl acrylate, stearyl acrylate, behenyl acrylate and melissyl acrylate and methacrylate versions thereof. Mixtures of two or three or more long chain acrylic esters may be successfully polymerized with one of the carboxylic monomers. Other comonomers include olefins, including alpha olefins, vinyl ethers, vinyl esters, and mixtures thereof.

Other vinylidene monomers, including the acrylic nitriles, may also be used as absorption-promoting agents within the methods and compositions of the invention to enhance delivery and absorption of interferon-α and other biologically active agent(s), including to enhance delivery of the active agent(s) to a target tissue or compartment in the subject (e.g., the systemic circulation). Useful alpha, beta-olefinically unsaturated nitriles are preferably monoolefinically unsaturated nitriles having from 3 to 10 carbon atoms such as acrylonitrile, methacrylonitrile, and the like. Most preferred are acrylonitrile and methacrylonitrile. Acrylic amides containing from 3 to 35 carbon atoms including monoolefinically unsaturated amides also may be used. Representative amides include acrylamide, methacrylamide, N-t-butyl acrylamide, N-cyclohexyl acrylamide, higher alkyl amides, where the alkyl group on the nitrogen contains from 8 to 32 carbon atoms, acrylic amides including N-alkylol amides of alpha, beta-olefinically unsaturated carboxylic acids including those having from 4 to 10 carbon atoms such as N-methylol acrylamide, N-propanol acrylamide, N-methylol methacrylamide, N-methylol maleimide, N-methylol maleamic acid esters, N-methylol-p-vinyl benzamide, and the like.

Yet additional useful absorption promoting materials are alpha-olefins containing from 2 to 18 carbon atoms, more preferably from 2 to 8 carbon atoms; dienes containing from 4 to 10 carbon atoms; vinyl esters and allyl esters such as vinyl acetate; vinyl aromatics such as styrene, methyl styrene and chloro-styrene; vinyl and allyl ethers and ketones such as vinyl methyl ether and methyl vinyl ketone; chloroacrylates; cyanoalkyl acrylates such as alpha-cyanomethyl acrylate, and the alpha-, beta-, and gamma-cyanopropyl acrylates; alkoxyacrylates such as methoxy ethyl acrylate; haloacrylates as chloroethyl acrylate; vinyl halides and vinyl chloride, vinylidene chloride and the like; divinyls, diacrylates and other polyfunctional monomers such as divinyl ether, diethylene glycol diacrylate, ethylene glycol dimethacrylate, methylene-bis-acrylamide, allylpentaerythritol, and the like; and bis (beta-haloalkyl) alkenyl phosphonates such as bis(beta-chloroethyl) vinyl phosphonate and the like as are known to those skilled in the art. Copolymers wherein the carboxy containing monomer is a minor constituent, and the other vinylidene monomers present as major components are readily prepared in accordance with the methods disclosed herein.

When hydrogels are employed as absorption promoting agents within the invention, these may be composed of synthetic copolymers from the group of acrylic and methacrylic acids, acrylamide, methacrylamide, hydroxyethylacrylate (HEA) or methacrylate (HEMA), and vinylpyrrolidones which are water interactive and swellable. Specific illustrative examples of useful polymers, especially for the delivery of peptides or proteins, are the following types of polymers:

(meth)acrylamide and 0.1 to 99 wt. % (meth)acrylic acid; (meth)acrylamides and 0.1-75 wt % (meth)acryloxyethyl trimethyammonium chloride; (meth)acrylamide and 0.1-75 wt % (meth)acrylamide; acrylic acid and 0.1-75 wt % alkyl(meth)acrylates;

(meth)acrylamide and 0.1-75 wt % AMPS.RTM. (trademark of Lubrizol Corp.);

(meth)acrylamide and 0 to 30 wt % alkyl(meth)acrylamides and 0.1-75 wt % AMPS.RTM.; (meth)acrylamide and 0.1-99 wt. % HEMA; (metb)acrylamide and 0.1 to 75 wt % HEMA and 0.1 to 99%(meth)acrylic acid; (meth)acrylic acid and 0.1-99 wt % HEMA; 50 mole % vinyl ether and 50 mole % maleic anhydride;

(meth)acrylamide and 0.1 to 75 wt % (meth)acryloxyalky dimethyl benzylammonium chloride; (meth)acrylamide and 0.1 to 99 wt % vinyl pyrrolidone; (meth)acrylamide and 50 wt % vinyl pyrrolidone and 0.1-99.9 wt % (meth)acrylic acid; (meth)acrylic acid and 0.1 to 75 wt % AMPS.RTM. and 0.1-75 wt % alkyl(meth)acrylamide. In the above examples, alkyl means C₁ to C₃₀, preferably C₁ to C₂₂, linear and branched and C₄ to C₁₆ cyclic; where (meth) is used, it means that the monomers with and without the methyl group are included. Other very useful hydrogel polymers are swellable, but insoluble versions of poly(vinyl pyrrolidone) starch, carboxymethyl cellulose and polyvinyl alcohol.

Additional polymeric hydrogel materials useful within the invention include (poly) hydroxyalkyl (meth)acrylate: anionic and cationic hydrogels: poly(electrolyte) complexes; poly(vinyl alcohols) having a low acetate residual: a swellable mixture of crosslinked agar and crosslinked carboxymethyl cellulose: a swellable composition comprising methyl cellulose mixed with a sparingly crosslinked agar; a water swellable copolymer produced by a dispersion of finely divided copolymer of maleic anhydride with styrene, ethylene, propylene, or isobutylene; a water swellable polymer of N-vinyl lactams; swellable sodium salts of carboxymethyl cellulose; and the like.

Other gelable, fluid imbibing and retaining polymers useful for forming the hydrophilic hydrogel for mucosal delivery of biologically active agents within the invention include pectin; polysaccharides such as agar, acacia, karaya, tragacenth, algins and guar and their crosslinked versions; acrylic acid polymers, copolymers and salt derivatives, polyacrylamides; water swellable indene maleic anhydride polymers; starch graft copolymers; acrylate type polymers and copolymers with water absorbability of about 2 to 400 times its original weight; diesters of polyglucan; a mixture of crosslinked poly(vinyl alcohol) and poly(N-vinyl-2-pyrrolidone); polyoxybutylene-polyethylene block copolymer gels; carob gum; polyester gels; poly urea gels; polyether gels; polyamide gels; polyimide gels; polypeptide gels; polyamino acid gels; poly cellulosic gels; crosslinked indene-maleic anhydride acrylate polymers; and polysaccharides.

Synthetic hydrogel polymers for use within the invention may be made by an infinite combination of several monomers in several ratios. The hydrogel can be crosslinked and generally possesses the ability to imbibe and absorb fluid and swell or expand to an enlarged equilibrium state. The hydrogel typically swells or expands upon delivery to the nasal mucosal surface, absorbing about 2-5, 5-10, 10-50, up to 50-100 or more times fold its weight of water. The optimum degree of swellability for a given hydrogel will be determined for different biologically active agents depending upon such factors as molecular weight, size, solubility and diffusion characteristics of the active agent carried by or entrapped or encapsulated within the polymer, and the specific spacing and cooperative chain motion associated with each individual polymer.

Hydrophilic polymers useful within the invention are water insoluble but water swellable. Such water swollen polymers as typically referred to as hydrogels or gels. Such gels may be conveniently produced from water soluble polymer by the process of crosslinking the polymers by a suitable crosslinking agent. However, stable hydrogels may also be formed from specific polymers under defined conditions of pH, temperature and/or ionic concentration, according to know methods in the art. Typically the polymers are cross-linked, that is, cross-linked to the extent that the polymers possess good hydrophilic properties, have improved physical integrity (as compared to non cross-linked polymers of the same or similar type) and exhibit improved ability to retain within the gel network both the biologically active agent of interest and additional compounds for coadministration therewith such as a cytokine or enzyme inhibitor, while retaining the ability to release the active agent(s) at the appropriate location and time.

Generally hydrogel polymers for use within the invention are crosslinked with a difunctional cross-linking in the amount of from 0.01 to 25 weight percent, based on the weight of the monomers forming the copolymer, and more preferably from 0.1 to 20 weight percent and more often from 0. 1 to 15 weight percent of the crosslinking agent. Another useful amount of a crosslinking agent is 0.1 to 10 weight percent. Tri, tetra or higher multifunctional crosslinking agents may also be employed. When such reagents are utilized, lower amounts may be required to attain equivalent crosslinking density, i.e., the degree of crosslinking, or network properties that are sufficient to contain effectively the biologically active agent(s).

The crosslinks can be covalent, ionic or hydrogen bonds with the polymer possessing the ability to swell in the presence of water containing fluids. Such crosslinkers and crosslinking reactions are known to those skilled in the art and in many cases are dependent upon the polymer system. Thus a crosslinked network may be formed by free radical copolymerization of unsaturated monomers. Polymeric hydrogels may also be formed by crosslinking preformed polymers by reacting functional groups found on the polymers such as alcohols, acids, amines with such groups as glyoxal, formaldehyde or glutaraldehyde, bis anhydrides and the like.

The polymers also may be cross-linked with any polyene, e.g. decadiene or trivinyl cyclohexane; acrylamides, such as N,N-methylene-bis (acrylamide); polyfunctional acrylates, such as trimethylol propane triacrylate; or polyfunctional vinylidene monomer containing at least 2 terminal CH.sub.2<groups, including, for example, divinyl benzene, divinyl naphthalene, allyl acrylates and the like. In certain embodiments, cross-linking monomers for use in preparing the copolymers are polyalkenyl polyethers having more than one alkenyl ether grouping per molecule, which may optionally possess alkenyl groups in which an olefinic double bond is present attached to a terminal methylene grouping (e.g., made by the etherification of a polyhydric alcohol containing at least 2 carbon atoms and at least 2 hydroxyl groups). Compounds of this class may be produced by reacting an alkenyl halide, such as allyl chloride or allyl bromide, with a strongly alkaline aqueous solution of one or more polyhydric alcohols. The product may be a complex mixture of polyethers with varying numbers of ether groups. Efficiency of the polyether cross-linking agent increases with the number of potentially polymerizable groups on the molecule. Typically, polyethers containing an average of two or more alkenyl ether groupings per molecule are used. Other cross-linking monomers include for example, diallyl esters, dimethallyl ethers, allyl or methallyl acrylates and acrylamides, tetravinyl silane, polyalkenyl methanes, diacrylates, and dimethacrylates, divinyl compounds such as divinyl benzene, polyallyl phosphate, diallyloxy compounds and phosphite esters and the like. Typical agents are allyl pentaerythritol, allyl sucrose, trimethylolpropane triacrylate, 1 ,6-hexanediol diacrylate, trimethylolpropane diallyl ether, pentaerythritol triacrylate, tetramethylene dimethacrylate, ethylene diacrylate, ethylene dimethacrylate, triethylene glycol dimethacrylate, and the like. Allyl pentaerythritol, trimethylolpropane diallylether and allyl sucrose provide suitable polymers. When the cross-linking agent is present, the polymeric mixtures usually contain between about 0.01 to 20 weight percent, e.g., 1%, 5%, or 10% or more by weight of cross-linking monomer based on the total of carboxylic acid monomer, plus other monomers.

In more detailed aspects of the invention, mucosal delivery of interferon-α and other biologically active agents disclosed herein, is enhanced by retaining the active agent(s) in a slow-release or enzymatically or physiologically protective carrier or vehicle, for example a hydrogel that shields the active agent from the action of the degradative enzymes. In certain embodiments, the active agent is bound by chemical means to the carrier or vehicle, to which may also be admixed or bound additional agents such as enzyme inhibitors, cytokines, etc. The active agent may alternately be immobilized through sufficient physical entrapment within the carrier or vehicle, e.g., a polymer matrix.

Polymers such as hydrogels useful within the invention may incorporate functional linked agents such as glycosides chemically incorporated into the polymer for enhancing intranasal bioavailability of active agents formulated therewith. Examples of such glycosides are glucosides, fructosides, galactosides, arabinosides, mannosides and their alkyl substituted derivatives and natural glycosides such as arbutin, phlorizin, amygdalin, digitonin, saponin, and indican. There are several ways in which a typical glycoside may be bound to a polymer. For example, the alkyl group from a hydrogel polymer to form an ether may replace the hydrogen of the hydroxyl groups of a glycoside or other similar carbohydrate. Also, the hydroxyl groups of the glycosides may be reacted to esterify the carboxyl groups of a polymeric hydrogel to form polymeric esters in situ. Another approach is to employ condensation of acetobromoglucose with cholest-5-en-3beta-ol on a copolymer of maleic acid. N-substituted polyacrylamides can be synthesized by the reaction of activated polymers with omega-aminoalkylglycosides: (1) (carbohydrate-spacer) (n)-polyacrylamide, ‘pseudopolysaccharides’; (2) (carbohydrate spacer) (n)-phosphatidylethanolamine(m)-polyacrylamide, neoglycolipids, derivatives of phosphatidylethanolamine; (3) (carbohydrate-spacer)(n)-biotin(m)-polyacrylamide. These biotinylated derivatives may attach to lectins on the mucosal surface to facilitate absorption of the biologically active agent(s), e.g., a polymer-encapsulated interferon-α.

Within more detailed aspects of the invention, interferon-α and/or other biologically active agents, disclosed herein, optionally including secondary active agents such as protease inhibitor(s), cytokine(s), additional modulator(s) of intercellular junctional physiology, etc., are modified and bound to a polymeric carrier or matrix. For example, this may be accomplished by chemically binding a peptide or protein active agent and other optional agent(s) within a crosslinked polymer network. It is also possible to chemically modify the polymer separately with an interactive agent such as a glycosidal containing molecule. In certain aspects, the biologically active agent(s), and optional secondary active agent(s), may be functionalized, i.e., wherein an appropriate reactive group is identified or is chemically added to the active agent(s). Most often an ethylenic polymerizable group is added, and the functionalized active agent is then copolymerized with monomers and a crosslinking agent using a standard polymerization method such as solution polymerization (usually in water), emulsion, suspension or dispersion polymerization. Often, the functionalizing agent is provided with a high enough concentration of functional or polymerizable groups to insure that several sites on the active agent(s) are functionalized. For example, in a polypeptide comprising 16 amine sites, it is generally desired to functionalize at least 2, 4, 5, 7, and up to 8 or more of said sites.

After functionalization, the functionalized active agent(s) is/are mixed with monomers and a crosslinking agent that comprise the reagents from which the polymer of interest is formed. Polymerization is then induced in this medium to create a polymer containing the bound active agent(s). The polymer is then washed with water or other appropriate solvents and otherwise purified to remove trace unreacted impurities and, if necessary, ground or broken up by physical means such as by stirring, forcing it through a mesh, ultrasonication or other suitable means to a desired particle size. The solvent, usually water, is then removed in such a manner as to not denature or otherwise degrade the active agent(s). One desired method is lyophilization (freeze drying) but other methods are available and may be used (e.g., vacuum drying, air drying, spray drying, etc.).

To introduce polymerizable groups in peptides, proteins and other active agents within the invention, it is possible to react available amino, hydroxyl, thiol and other reactive groups with electrophiles containing unsaturated groups. For example, unsaturated monomers containing N-hydroxy succinimidyl groups, active carbonates such as p-nitrophenyl carbonate, trichlorophenyl carbonates, tresylate, oxycarbonylimidazoles, epoxide, isocyanates and aldehyde, and unsaturated carboxymethyl azides and unsaturated orthopyridyl-disulfide belong to this category of reagents. Illustrative examples of unsaturated reagents are allyl glycidyl ether, allyl chloride, allylbromide, allyl iodide, acryloyl chloride, allyl isocyanate, allylsulfonyl chloride, maleic anhydride, copolymers of maleic anhydride and allyl ether, and the like.

All of the lysine active derivatives, except aldehyde, can generally react with other amino acids such as imidazole groups of histidine and hydroxyl groups of tyrosine and the thiol groups of cystine if the local environment enhances nucleophilicity of these groups. Aldehyde containing functionalizing reagents are specific to lysine. These types of reactions with available groups from lysines, cysteines, tyrosine have been extensively documented in the literature and are known to those skilled in the art.

In the case of biologically active agents that contain amine groups, it is convenient to react such groups with an acyloyl chloride, such as acryloyl chloride, and introduce the polymerizable acrylic group onto the reacted agent. Then during preparation of the polymer, such as during the crosslinking of the copolymer of acrylamide and acrylic acid, the functionalized active agent, through the acrylic groups, is attached to the polymer and becomes bound thereto.

In additional aspects of the invention, biologically active agents, including peptides, proteins, other molecules which are bioactive in vivo, are conjugation-stabilized by covalently bonding one or more active agent(s) to a polymer incorporating as an integral part thereof both a hydrophilic moiety, e.g., a linear polyalkylene glycol, a lipophilic moiety (see, e.g., U.S. Pat. No. 5,681,81 1, incorporated herein by reference). In one aspect, a biologically active agent is covalently coupled with a polymer comprising (i) a linear polyalkylene glycol moiety and (ii) a lipophilic moiety, wherein the active agent, linear polyalkylene glycol moiety, and the lipophilic moiety are conformnationally arranged in relation to one another such that the active therapeutic agent has an enhanced in vivo resistance to enzymatic degradation (i.e., relative to its stability under similar conditions in an unconjugated form devoid of the polymer coupled thereto). In another aspect, the conjugation-stabilized formulation has a three-dimensional conformation comprising the biologically active agent covalently coupled with a polysorbate complex comprising (i) a linear polyalkylene glycol moiety and (ii) a lipophilic moiety, wherein the active agent, the linear polyalkylene glycol moiety and the lipophilic moiety are conformationally arranged in relation to one another such that (a) the lipophilic moiety is exteriorly available in the three-dimensional conformation, and (b) the active agent in the composition has an enhanced in vivo resistance to enzymatic degradation.

In a further related aspect, a multiligand conjugated complex is provided which comprises a biologically active agent covalently coupled with a triglyceride backbone moiety through a polyalkylene glycol spacer group bonded at a carbon atom of the triglyceride backbone moiety, and at least one fatty acid moiety covalently attached either directly to a carbon atom of the triglyceride backbone moiety or covalently joined through a polyalkylene glycol spacer moiety (see, e.g., U.S. Pat. No. 5,681,811,). In such a multiligand conjugated therapeutic agent complex, the alpha' and beta carbon atoms of the triglyceride bioactive moiety may have fatty acid moieties attached by covalently bonding either directly thereto, or indirectly covalently bonded thereto through polyalkylene glycol spacer moieties. Alternatively, a fatty acid moiety may be covalently attached either directly or through a polyalkylene glycol spacer moiety to the alpha and alpha' carbons of the triglyceride backbone moiety, with the bioactive therapeutic agent being covalently coupled with the gamma-carbon of the triglyceride backbone moiety, either being directly covalently bonded thereto or indirectly bonded thereto through a polyalkylene spacer moiety. It will be recognized that a wide variety of structural, compositional, and conformational forms are possible for the multiligand conjugated therapeutic agent complex comprising the triglyceride backbone moiety, within the scope of the invention. It is further noted that in such a multiligand conjugated therapeutic agent complex, the biologically active agent(s) may advantageously be covalently coupled with the triglyceride modified backbone moiety through alkyl spacer groups, or alternatively other acceptable spacer groups, within the scope of the invention. As used in such context, acceptability of the spacer group refers to steric, compositional, and end use application specific acceptability characteristics.

In yet additional aspects of the invention, a conjugation-stabilized complex is provided which comprises a polysorbate complex comprising a polysorbate moiety including a triglyceride backbone having covalently coupled to alpha, alpha' and beta carbon atoms thereof functionalizing groups including (i) a fatty acid group; and (ii) a polyethylene glycol group having a biologically active agent or moiety covalently bonded thereto, e.g., bonded to an appropriate functionality of the polyethylene glycol group (see, e.g., U.S. Pat. No. 5,681,811,). Such covalent bonding may be either direct, e.g., to a hydroxy terminal functionality of the polyethylene glycol group, or alternatively, the covalent bonding may be indirect, e.g., by reactively capping the hydroxy terminus of the polyethylene glycol group with a terminal carboxy functionality spacer group, so that the resulting capped polyethylene glycol group has a terminal carboxy functionality to which the biologically active agent or moiety may be covalently bonded.

In yet additional aspects of the invention, a stable, aqueously soluble, conjugation-stabilized complex is provided which comprises one or more interferon-α and/or other biologically active agent(s)+disclosed herein covalently coupled to a physiologically compatible polyethylene glycol (PEG) modified glycolipid moiety. In such complex, the biologically active agent(s) may be covalently coupled to the physiologically compatible PEG modified glycolipid moiety by a labile covalent bond at a free amino acid group of the active agent, wherein the labile covalent bond is scissionable in vivo by biochemical hydrolysis and/or proteolysis. The physiologically compatible PEG modified glycolipid moiety may advantageously comprise a polysorbate polymer, e.g., a polysorbate polymer comprising fatty acid ester groups selected from the group consisting of monopalmitate, dipalmitate, monolaurate, dilaurate, trilaurate, monoleate, dioleate, trioleate, monostearate, distearate, and tristearate. In such complex, the physiologically compatible PEG modified glycolipid moiety may suitably comprise a polymer selected from the group consisting of polyethylene glycol ethers of fatty acids, and polyethylene glycol esters of fatty acids, wherein the fatty acids for example comprise a fatty acid selected from the group consisting of lauric, palmitic, oleic, and stearic acids.

Bioadhesive Delivery Vehicles and Methods

In certain aspects of the invention, the combinatorial formulations and/or coordinate administration methods herein incorporate an effective amount of a nontoxic bioadhesive as an adjunct compound or carrier to enhance mucosal delivery of interferon-α. Bioadhesive agents in this context exhibit general or specific adhesion to one or more components or surfaces of the targeted mucosa. The bioadhesive maintains a desired concentration gradient of interferon-α into or across the mucosa to ensure penetration of even large molecules (e.g., peptides and proteins) into or through the mucosal epithelium. Typically, employment of a bioadhesive within the methods and compositions of the invention yields a two- to five- fold, often a five- to ten-fold increase in permeability for interferon-α into or through the mucosal epithelium. This enhancement of epithelial permeation often permits effective transmucosal delivery of large macromolecules, for example to the basal portion of the nasal epithelium or into the adjacent extracellular compartments or the systemic circulation.

This enhanced delivery provides for greatly improved effectiveness of delivery of bioactive therapeutic species. These results will depend in part on the hydrophilicity of the compound, whereby greater penetration will be achieved with hydrophilic species compared to water insoluble compounds. In addition to these effects, employment of bioadhesives to enhance drug persistence at the mucosal surface can elicit a reservoir mechanism for protracted drug delivery, whereby compounds not only penetrate across the mucosal tissue but also back-diffuse toward the mucosal surface once the material at the surface is depleted.

Typically, mucoadhesive polymers for use within the invention are natural or synthetic macromolecules which adhere to wet mucosal tissue surfaces by complex, but non-specific, mechanisms. In addition to these mucoadhesive polymers, the invention also provides methods and compositions incorporating bioadhesives that adhere directly to a cell surface, rather than to mucus, by means of specific, including receptor-mediated, interactions. One example of bioadhesives that function in this specific manner is the group of compounds known as lectins. These are glycoproteins with an ability to specifically recognize and bind to sugar molecules, e.g. glycoproteins or glycolipids, which form part of intranasal epithelial cell membranes and can be considered as “lectin receptors”.

Exemplary mucoadhesive polymers for use within the invention, for example chitosan, enhance the permeability of mucosal epithelia even when they are applied as an aqueous solution or gel. In one study, absorption of the peptide drugs insulin and interferon-α, and the hydrophilic compound phenol red, from an aqueous gel base of poly(acrylic acid) was reported after rectal, vaginal and nasal administration. Another mucoadhesive polymer reported to directly affect epithelial permeability is hyaluronic acid. In particular, hyaluronic acid gel formulation reportedly enhanced nasal absorption of vasopressin and some of its analogues. Hyaluronic acid was also reported to increase the absorption of insulin from the conjunctiva in diabetic dogs. Ester derivatives of hyaluronic acid in the form of lyophilized microspheres were described as a nasal delivery system for insulin.

A particularly useful bioadhesive agent within the coordinate administration, and/or combinatorial formulation methods and compositions of the invention is chitosan, as well as its analogs and derivatives. Chitosan is a non-toxic, biocompatible and biodegradable polymer that is widely used for pharmaceutical and medical applications because of its favorable properties of low toxicity and good biocompatibility. It is a natural polyaminosaccharide prepared from chitin by N-deacetylation with alkali.

As used within the methods and compositions of the invention, chitosan increases the retention of interferon-α and other biologically active agents disclosed herein at a mucosal site of application.

As further provided herein, the methods and compositions of the invention will optionally include a novel chitosan derivative or chemically modified form of chitosan. One such novel derivative for use within the invention is denoted as a β-[1→4]-2-guanidino-2-deoxy-D-glucose polymer (poly-GuD). Chitosan is the N-deacetylated product of chitin, a naturally occurring polymer that has been used extensively to prepare microspheres for oral and intra-nasal formulations. The chitosan polymer has also been proposed as a soluble carrier for parenteral drug delivery. Within one aspect of the invention, o-methylisourea is used to convert a chitosan amine to its guanidinium moiety. The guanidinium compound is prepared, for example, by the reaction between equi-normal solutions of chitosan and o-methylisourea at pH above 8.0.

The guanidinium product is -[14]-guanidino-2-deoxy-D-glucose polymer. It is abbreviated as Poly-GuD in this context (Monomer F.W. of Amine in Chitosan=161;

Monomer F.W. of Guanidinium in Poly-GuD =203).

One exemplary Poly-GuD preparation method for use within the invention involves the following protocol.

Solutions:

Preparation of 0.5% Acetic Acid Solution (0.088N):

Pipette 2.5 mL glacial acetic acid into a 500 mL volumetric flask, dilute to volume with purified water.

Preparation of 2N NaOH Solution:

Transfer about 20 g NaOH pellets into a beaker with about 150 mL of purified water. Dissolve and cool to room temperature. Transfer the solution into a 250-mL volumetric flask, dilute to volume with purified water.

Preparation of O-methylisourea Sulfate (0.4N urea group equivalent):

Transfer about 493 mg of O-methylisourea sulfate into a 10-mL volumetric flask, dissolve and dilute to volume with purified water.

The pH of the solution is 4.2

Preparation of Barium Chloride Solution (0.2M):

Transfer about 2.086 g of Barium chloride into a 50-mL volumetric flask, dissolve and dilute to volume with purified water.

Preparation of Chitosan Solution (0.06N amine equivalent):

Transfer about 100 mg Chitosan into a 50 mL beaker, add 10 mL 0.5% Acetic Acid (0.088 N). Stir to dissolve completely.

The pH of the solution is about 4.5

Preparation of O-methylisourea Chloride Solution (0.2N urea group equivalent):

Pipette 5.0 mL of O-methylisourea sulfate solution (0.4 N urea group equivalent) and 5 mL of 0.2M Barium chloride solution into a beaker. A precipitate is formed. Continue to mix the solution for additional 5 minutes. Filter the solution through 0.45 m filter and discard the precipitate. The concentration of O-methylisourea chloride in the supernatant solution is 0.2 N urea group equivalent.

The pH of the solution is 4.2.

Procedure:

Add 1.5 mL of 2 N NaOH to 10 mL of the chitosan solution (0.06N amine equivalent) prepared as described in Section 2.5. Adjust the pH of the solution with 2N NaOH to about 8.2 to 8.4. Stir the solution for additional 10 minutes. Add 3.0 mL O-methylisourea chloride solution (0.2N urea group equivalent) prepared as described above. Stir the solution overnight.

Adjust the pH of solution to 5.5 with 0.5% Acetic Acid (0.088N).

Dilute the solution to a final volume of 25 mL using purified water.

The Poly-GuD concentration in the solution is 5 mg/mL, equivalent to 0.025 N (guanidium group).

In summary, the foregoing bioadhesive agents are useful in the combinatorial formulations and coordinate administration methods of the instant invention, which optionally incorporate an effective amount and form of a bioadhesive agent to prolong persistence or otherwise increase mucosal absorption of interferon-α. The bioadhesive agents may be coordinately administered as adjunct compounds or as additives within the combinatorial formulations of the invention, for example, with benzethonium chloride or chlorobutanol. In certain embodiments, the bioadhesive agent acts as a “pharmaceutical glue”, whereas in other embodiments adjunct delivery or combinatorial formulation of the bioadhesive agent serves to intensify contact of interferon-α with the nasal mucosa, in some cases by promoting specific receptor-ligand interactions with epithelial cell “receptors”, and in others by increasing epithelial permeability to significantly increase the drug concentration gradient measured at a target site of delivery (e.g., the CNS or in the systemic circulation). Yet additional bioadhesive agents for use within the invention act as enzyme (e.g., protease) inhibitors to enhance the stability of mucosally administered biotherapeutic agents, for example, interferon-α, delivered coordinately or in a combinatorial formulation with the bioadhesive agent.

Liposomes and Micellar Delivery Vehicles

The coordinate administration methods and combinatorial formulations of the instant invention optionally incorporate effective lipid or fatty acid based carriers, processing agents, or delivery vehicles, to provide improved formulations for mucosal delivery interferon-α and other biologically active agents. For example, a variety of formulations and methods are provided for mucosal delivery which comprise one or more of these active agents, such as a peptide or protein, admixed or encapsulated by, or coordinately administered with, a liposome, mixed micellar carrier, or emulsion, to enhance chemical and physical stability and increase the half life of the biologically active agents (e.g., by reducing susceptibility to proteolysis, chemical modification and/or denaturation) upon mucosal delivery.

Within certain aspects of the invention, specialized delivery systems for biologically active agents comprise small lipid vesicles known as liposomes (see, e.g., Chonn et al., Curr. Opin. Biotechnol. 6: 698-708, 1995; Lasic, Trends Biotechnol. 16: 307-321, 1998; and Gregoriadis, Trends Biotechnol. 13: 527-537, 1995,). These are typically made from natural, biodegradable, non-toxic, and non-immunogenic lipid molecules, and can efficiently entrap or bind drug molecules, including peptides and proteins, into, or onto, their membranes. A variety of methods are available for preparing liposomes for use within the invention (e.g., as described in Szoka et al., Ann. Rev. Biophys. Bioeng. 9: 467, 1980; and U.S. Pat. Nos. 4,235,871, 4,501,728, and 4,837,028,). For use with liposome delivery, the biologically active agent is typically entrapped within the liposome, or lipid vesicle, or is bound to the outside of the vesicle. Several strategies have been devised to increase the effectiveness of liposome-mediated delivery by targeting liposomes to specific tissues and specific cell types. Liposome formulations, including those containing a cationic lipid, have been shown to be safe and well tolerated in human patients.

Like liposomes, unsaturated long chain fatty acids, which also have enhancing activity for mucosal absorption, can form closed vesicles with bilayer-like structures (so called “ufasomes”). These can be formed, for example, using oleic acid to entrap biologically active peptides and proteins for mucosal, e.g., intranasal, delivery within the invention.

Additional delivery vehicles for use within the invention include long and medium chain fatty acids, as well as surfactant mixed micelles with fatty acids [see, e.g., Muranishi, Crit. Rev. Ther. Drug Carrier Syst. 7: 1-33, (1990)]. Most naturally occurring lipids in the form of esters have important implications with regard to their own transport across mucosal surfaces. Free fatty acids and their monoglycerides which have polar groups attached have been demonstrated in the form of mixed micelles to act on the intestinal barrier as penetration enhancers. This discovery of barrier modifying function of free fatty acids (carboxylic acids with a chain length varying from 12 to 20 carbon atoms) and their polar derivatives has stimulated extensive research on the application of these agents as mucosal absorption enhancers.

For use within the methods of the invention, long chain fatty acids, especially fusogenic lipids (unsaturated fatty acids and monoglycerides such as oleic acid, linoleic acid, linoleic acid, monoolein, etc.) provide useful carriers to enhance mucosal delivery of interferon-α and other biologically active agents disclosed herein. Medium chain fatty acids (C6 to C12) and monoglycerides have also been shown to have enhancing activity in intestinal drug absorption and can be adapted for use within the mucosal delivery formulations and methods of the invention. In addition, sodium salts of medium and long chain fatty acids are effective delivery vehicles and absorption-enhancing agents for mucosal delivery of biologically active agents within the invention. Thus, fatty acids can be employed in soluble forms of sodium salts or by the addition of non-toxic surfactants, e.g., polyoxyethylated hydrogenated castor oil, sodium taurocholate, etc. Mixed micelles of naturally occurring unsaturated long chain fatty acids (oleic acid or linoleic acid) and their monoglycerides with bile salts have been shown to exhibit absorption-enhancing abilities that are basically harmless to the intestinal mucosa (see, e.g., Muranishi, Pharm. Res. 2: 108-118, 1985; and Crit. Rev. Ther. drug carrier Syst. 7: 1-33, 1990,). Other fatty acid and mixed micellar preparations that are useful within the invention include, but are not limited to, Na caprylate (C8), Na caprate (C 10), Na laurate (C12) or Na oleate (C18), optionally combined with bile salts, such as glycocholate and taurocholate.

Degradative Enzyme Inhibitory Agents and Methods

A major drawback to effective mucosal delivery of biologically active agents, including interferon-α peptides, is that they may be subject to degradation by mucosal enzymes. The oral route of administration of therapeutic compounds is particularly problematic, because in addition to proteolysis in the stomach, the high acidity of the stomach destroys many active and inactive components of mucosal delivery formulations before they reach an intended target site of drug action. Further impairment of activity occurs by the action of gastric and pancreatic enzymes, and exo and endopeptidases in the intestinal brush border membrane, and by metabolism in the intestinal mucosa where a penetration barrier substantially blocks passage of the active agent across the mucosa. In addition to their susceptibility to enzymatic degradation, many therapeutic compounds, particularly relatively low molecular weight proteins, and peptides, introduced into the circulation, are cleared quickly from mammalian subjects by the kidneys.

Attempts to overcome the so-called enzymatic barrier to drug delivery include the use of liposomes, Takeuchi et al., Pharm. Res., 13: 896-901, 1996, and nanoparticles, Mathiowitz et al., Nature., 386: 410-4, 1997, that reportedly provide protection for incorporated insulin towards an enzymatic attack and the development of delivery systems targeting to the colon, where the enzymatic activity is comparatively low. Rubenstein et al., J. Control Rel., 46: 59-73, 1997. In addition, co-administration of protease inhibitors has been reported in various studies to improve the oral bioavailability of insulin.

More recent research efforts in the area of protease inhibition for enhanced delivery of biotherapeutic compounds, including peptide and protein therapeutics, has focused on covalent immobilization of enzyme inhibitors on mucoadhesive polymers used as drug carrier matrices. Bemkop-Schnurch et al., Drug Dev. Ind. Pharm., 23: 733-40, 1997; Bernkop-Schnurch et al., J. Control. Rel., 47: 113-21, 1997; Bernkop-Schnurch et al., J. Drug Targ.. 7: 55-63, 1999. In conjunction with these teachings, the invention provides in more detailed aspects an enzyme inhibitor formulated with a common carrier or vehicle for mucosal delivery of interferon-α peptides and other biologically active peptides, analogs and mimetics, optionally to be administered coordinately one or more additional biologically active or delivery-enhancing agents. Optionally, the enzyme inhibitor is covalently linked to the carrier or vehicle. In certain embodiments, the carrier or vehicle is a biodegradable polymer, for example, a bioadhesive polymer. Thus, for example, a protease inhibitor, such as Bowman-Birk inhibitor (BBI), displaying an inhibitory effect towards trypsin and \acute{α}-chymotrypsin, Birk Y. Int. J. Pept. Protein Res., 25: 113-31, 1985, or elastatinal, an elastase-specific inhibitor of low molecular size, may be covalently linked to a mucoadhesive polymer as described herein. The resulting polymer-inhibitor conjugate exhibits substantial utility as a mucosal delivery vehicle for peptides and other biologically active agents formulated or delivered alone or in combination with other biologically active agents or additional delivery-enhancing agents.

Exemplary mucoadhesive polymer-enzyme inhibitor complexes that are useful within the mucosal delivery formulations and methods of the invention include, but are not limited to: Carboxymethylcellulose-pepstatin (with anti-pepsin activity); Poly(acrylic acid)-Bowman-Birk inhibitor (anti-chymotrypsin); Poly(acrylic acid)-chymostatin (anti-chymotrypsin); Poly(acrylic acid)-elastatinal (anti-elastase); Carboxymethylcellulose-elastatinal (anti-elastase); Polycarbophil—elastatinal (anti-elastase); Chitosan—antipain (anti-trypsin); Poly(acrylic acid)—bacitracin (anti-aminopeptidase N); Chitosan—EDTA (anti-aminopeptidase N, anti-carboxypeptidase A); Chitosan—EDTA—antipain (anti-trypsin, anti-chymotrypsin, anti-elastase). Bernkop-Schnüuirch, J. Control. Rel., 52: 1-16, 1998, incorporated herein by reference. As described in further detail below, certain embodiments of the invention will optionally incorporate a novel chitosan derivative or chemically modified form of chitosan. One such derivative for use within the invention is denoted as β-[1→4]-2-guanidino-2-deoxy-D-glucose polymer (poly-GuD).

Agents for Modulating Epithelial Junction Structure and/or Physiology

The present invention provides novel pharmaceutical compositions that include a biologically active agent and a permeabilizing agent effective to enhance mucosal delivery of the biologically active agent in a mammalian subject. The permeabilizing agent reversibly enhances mucosal epithelial paracellular transport, typically by modulating epithelial junctional structure and/or physiology at a mucosal epithelial surface in the subject. This effect typically involves inhibition by the permeabilizing agent of homotypic or heterotypic binding between epithelial membrane adhesive proteins of neighboring epithelial cells. Target proteins for this blockade of homotypic or heterotypic binding can be selected from various related junctional adhesion molecules (JAMs), occludins, or claudins.

In more detailed embodiments of the invention, the permeabilizing agent is a peptide or peptide analog or mimetic. Exemplary permeabilizing peptides comprise from about 4-25 contiguous amino acids of an extracellular domain of a mammalian JAM-1, JAM-2, or JAM-3 protein. Alternatively, the permeabilizing peptide may comprise from about 6-15 contiguous amino acids of an extracellular domain of a mammalian JAM-1, JAM-2, or JAM-3 protein. In additional embodiments, the permeabilizing peptide comprises from about 4-25 contiguous amino acids of an extracellular domain of a mammalian JAM-1, JAM-2, or JAM-3 protein, or a sequence of amino acids that exhibits at least 85% amino acid identity with a corresponding reference sequence of 4-25 contiguous amino acids of an extracellular domain of a mammalian JAM-1, JAM-2, or JAM-3 protein. In certain embodiments, the amino acid sequence of the permeabilizing peptide exhibits one or more amino acid substitutions, insertions, or deletions compared to the corresponding reference sequence of the mammalian JAM-1, JAM-2, or JAM-3 protein. For example, the permeabilizing peptide may exhibit one or more conservative amino acid substitutions compared to a corresponding reference sequence of a mammalian JAM-1, JAM-2, or JAM-3 protein. Such functional peptide analogs or variants may, for instance, have one or more amino acid mutations in comparison to a corresponding wild-type sequence of the same human JAM protein (e.g., human JAM-1), wherein the mutation(s) correspond to a divergent amino acid residue or sequence identified in a different human JAM protein (e.g., human JAM-2 or JAM-3) or in a homologous JAM protein found in a different species (e.g. murine, rat, or bovine JAM-1, JAM-2 or JAM-3 protein).

Within additional aspects of the invention, pharmaceutical compositions and methods are provided which employ a permeabilizing peptide comprising from about 4-25 contiguous amino acids of an extracellular domain of a mammalian occludin protein. In alternate embodiments, the permeabilizing peptide comprises from about 6-15 contiguous amino acids of an extracellular domain of a mammalian occludin protein. In certain aspects, the permeabilizing peptide comprises from about 4-25 contiguous amino acids of an extracellular domain of a mammalian occludin protein or comprises an amino acid sequence that exhibits at least 85% amino acid identity with a corresponding reference sequence of 4-25 contiguous amino acids of an extracellular domain of a mammalian occludin protein. In exemplary embodiments, the permeabilizing peptide exhibits one or more amino acid substitutions, insertions, or deletions compared to a corresponding reference sequence of the mammalian occludin protein. Often, such peptide “analogs” will exhibit one or more conservative amino acid substitutions compared to the corresponding reference sequence of the mammalian occludin protein. In related embodiments, the permeabilizing peptide is a human occludin peptide and the amino acid sequence of the permeabilizing peptide exhibits one or more amino acid mutations in comparison to a corresponding wild-type sequence of the same human occludin protein, wherein the mutation(s) correspond to a structural feature (e.g., a divergent, aligned residue or sequence of residues) identified in a different human occludin protein or a homologous occludin protein found in a different species.

Within other aspects of the invention, pharmaceutical compositions and methods are provided which employ a permeabilizing peptide comprising from about 4-25 contiguous amino acids of an extracellular domain of a mammalian claudin protein. In alternate embodiments, the permeabilizing peptide comprises from about 6-15 contiguous amino acids of an extracellular domain of a mammalian claudin protein. In certain aspects, the permeabilizing peptide comprises from about 4-25 contiguous amino acids of an extracellular domain of a mammalian claudin protein or comprises an amino acid sequence that exhibits at least 85% amino acid identity with a corresponding reference sequence of 4-25 contiguous amino acids of an extracellular domain of a mammalian claudin protein. In exemplary embodiments, the permeabilizing peptide exhibits one or more amino acid substitutions, insertions, or deletions compared to a corresponding reference sequence of the mammalian claudin protein. Often, such peptide “analogs” will exhibit one or more conservative amino acid substitutions compared to the corresponding reference sequence of the mammalian claudin protein. In related embodiments, the permeabilizing peptide is a human claudin peptide and the amino acid sequence of the permeabilizing peptide exhibits one or more amino acid mutations in comparison to a corresponding wild-type sequence of the same human claudin protein, wherein the mutation(s) correspond to a structural feature (e.g., a divergent, aligned residue or sequence of residues) identified in a different human claudin protein or a homologous claudin protein found in a different species.

In related aspects of the invention, the pharmaceutical composition includes the permeabilizing agent and one or more biologically active agent(s) selected from a small molecule drug, a peptide, a protein, and a vaccine agent.

In yet additional detailed embodiments, the invention provides permeabilizing peptides and peptide analogs and mimetics for enhancing mucosal epithelial paracellular transport. The subject peptides and peptide analogs and mimetics typically work within the compositions and methods of the invention by modulating epithelial junctional structure and/or physiology in a mammalian subject. In certain embodiments, the peptides and peptide analogs and mimetics effectively inhibit homotypic and/or heterotypic binding of an epithelial membrane adhesive protein selected from a junctional adhesion molecule (JAM), occludin, or claudin. In more detailed embodiments, the permeabilizing peptide or peptide analog comprises from about 4-25 contiguous amino acids of a wild-type sequence of an extracellular domain of a mammalian JAM-1, JAM-2, JAM-3, occludin or claudin protein, or an amino acid sequence that exhibits at least 85% amino acid identity with a corresponding reference sequence of about 4-25 contiguous amino acids of a wild-type sequence of an extracellular domain of a mammalian JAM-1, JAM-2, JAM-3, occludin or claudin protein. In exemplary embodiments, the permeabilizing peptide or peptide analog is a human JAM peptide (e.g., human JAM-1) having a wild-type amino acid sequence or exhibiting one or more amino acid mutations in comparison to a corresponding wild-type sequence of the same human JAM protein, wherein the mutation(s) correspond to a structural feature identified in a different human JAM protein or a homologous JAM protein found in a different species.

Further description related to these aspects of the invention are found in U.S. Patent Application entitled COMPOSITIONS AND METHODS FOR MODULATING PHYSIOLOGY OF EPITHELIAL JUNCTIONAL ADHESION MOLECULES FOR ENHANCED MUCOSAL DELIVERY OF THERAPEUTIC COMPOUNDS, Ser. No. 10/601,953, filed Jun. 24, 2003.

In addition to JAM, occludin and claudin peptides, proteins, analogs and mimetics, additional agents for modulating epithelial junctional physiology and/or structure are contemplated for use within the methods and formulations of the invention. Epithelial tight junctions are generally impermeable to molecules with radii of approximately 15 angstroms, unless treated with junctional physiological control agents that stimulate substantial junctional opening as provided within the instant invention. Among the “secondary” tight junctional regulatory components that will serve as useful targets for secondary physiological modulation within the methods and compositions of the invention, the ZO1-Z02 heterodimeric complex has shown itself amenable to physiological regulation by exogenous agents that can readily and effectively alter paracellular permeability in mucosal epithelia. On such agent that has been extensively studied is the bacterial toxin from Vibrio cholerae known as the “zonula occludens toxin” (ZOT). This toxin mediates increased intestinal mucosal permeability and causes disease symptoms including diarrhea in infected subjects (Fasano et al, Proc. Nat. Acad. Sci., USA 8: 5242-5246, 1991; Johnson et al, J. Clin. Microb. 31/3: 732-733, 1993; and Karasawa et al, FEBS Let. 106: 143-146, 1993, each incorporated herein by reference). When tested on rabbit ileal mucosa, ZOT increased the intestinal permeability by modulating the structure of intercellular tight junctions. More recently, it has been found that ZOT is capable of reversibly opening tight junctions in the intestinal mucosa (see, e.g., WO 96/37196; U.S. Pat. Nos. 5,945,510; 5,948,629; 5,912,323; 5,864,014; 5,827,534; 5,665,389, each incorporated herein by reference). It has also been reported that ZOT is capable of reversibly opening tight junctions in the nasal mucosa (U.S. Pat. No. 5,908,825, incorporated herein by reference). Thus, ZOT and other agents that modulate the ZO1-ZO2 complex will be combinatorially formulated or coordinately administered with one or more JAM, occludin and claudin pepfides, proteins, analogs and mimetics, and/or other biologically active agents disclosed herein. Within the methods and compositions of the invention, ZOT, as well as various analogs and mimetics of ZOT that function as agonists or antagonists of ZOT activity, are useful for enhancing intranasal delivery of biologically active agents-by increasing paracellular absorption into and across the nasal mucosa.

Pegylation

Additional methods and compositions provided within the invention involve chemical modification of biologically active peptides and proteins by covalent attachment of polymeric materials, for example dextrans, polyvinyl pyrrolidones, glycopeptides, polyethylene glycol and polyamino acids. The resulting conjugated peptides and proteins retain their biological activities and solubility for mucosal administration. In alternate embodiments, interferon-α peptides, proteins, analogs and mimetics, and other biologically active peptides and proteins, are conjugated to polyalkylene oxide polymers, particularly polyethylene glycols (PEG). U.S. Pat. No. 4,179,337, incorporated herein by reference. Numerous reports in the literature describe the potential advantages of pegylated peptides and proteins, which often exhibit increased resistance to proteolytic degradation, increased plasma half-life, increased solubility and decreased antigenicity and immunogenicity. Nucci, et al., Advanced Drug Deliver Reviews, 6: 133-155, 1991; Lu et al., Int. J. Peptide Protein Res., 43: 127-138, 1994, each incorporated herein by reference. A number of proteins, including L-asparaginase, strepto-kinase, insulin, interleukin-2, adenosine deamidase, L-asparaginase, interferon alpha 2b, superoxide dismutase, streptokinase, tissue plasminogen activator (tPA), urokinase, uricase, hemoglobin, TGF-beta, EGF, and other growth factors, have been conjugated to PEG and evaluated for their altered biochemical properties as therapeutics. Ho, et al., Drug Metabolism and Disposition 14: 349-352, 1986; Abuchowski et al., Prep. Biochem., 9: 205-211, 1979; and Rajagopaian et al., J. Clin. Invest., 75: 413-419, 1985, Nucci et al., Adv. Drug Delivery Rev., 4: 133-151, 1991, each incorporated herein by reference. Although the in vitro biological activities of pegylated proteins may be decreased, this loss in activity is usually offset by the increased in vivo half-life in the bloodstream. Nucci, et al., Advanced Drug Deliver Reviews, 6: 133-155, 1991, incorporated herein by reference. Accordingly, these and other polymer-coupled peptides and proteins exhibit enhanced properties, such as extended half-life and reduced immunogenicity, when administered mucoally according to the methods and formulations herein.

Several procedures have been reported for the attachment of PEG to proteins and peptides and their subsequent purification. Abuchowski et al., J. Biol. Chem., 252: 3582-3586,1977; Beauchamp etal., Anal. Biochem., 131: 25-33, 1983, each incorporated herein by reference. In addition, Lu et al., Int. J. Peptide Protein Res., 43: 127-138, 1994, incorporated herein by reference, describe various technical considerations and compare PEGylation procedures for proteins versus peptides. Katre et al., Proc. Natl. Acad. Sci. U.S.A., 84: 1487-1491, 1987; Becker et al., Makromol. Chem. Rapid Commun.,3: 217-223, 1982; Mutter et al., Makromol. Chem. Rapid Commun., 13: 151-157, 1992; Merrifield, R. B., J. Am. Chem. Soc., 85: 2149-2154, 1993; Lu et al., Peptide Res., 6: 142-146, 1993; Lee et al., Bioconjugate Chem., 10: 973-981, 1999, Nucci et al., Adv. Drug Deliv. Rev., 6: 133-151, 1991; Francis et al., J. Drug Targeting, 3: 321-340, 1996; Zalipsky, S., Bioconjugate Chem., 6: 150-165, 1995; Clark et al., J. Biol. Chem., 271: 21969-21977, 1996; Pettit et al., J. Biol. Chem., 272: 2312-2318, 1997; Delgado et al., Br. J. Cancer, 73: 175-182, 1996; Benhar et al., Bioconjugate Chem., 5: 321-326, 1994; Benhar et al., J. Biol. Chem., 269: 13398-13404, 1994; Wang et al., Cancer Res., 53: 4588-4594, 1993; Kinstler et al., Pharm. Res. 13: 996-1002, 1996, Filpula et al., Exp. Opin. Ther. Patents, 9: 231-245, 1999; Pelegrin et al., Hum. Gene Ther., 9: 2165-2175, 1998, each incorporated herein by reference.

Following these and other teachings in the art, the conjugation of biologically active peptides and proteins for with polyethyleneglycol polymers, is readily undertaken, with the expected result of prolonging circulating life and/or reducing immunogenicity while maintaining an acceptable level of activity of the PEGylated active agent. Amine-reactive PEG polymers for use within the invention include SC-PEG with molecular masses of 2000, 5000, 10000, 12000, and 20000; U-PEG- 10000; NHS-PEG-3400-biotin; T-PEG-5000; T-PEG-12000; and TPC-PEG-5000. Chemical conjugation chemistries for these polymers have been published. Zalipsky, S., Bioconiugate Chem., 6: 150-165, 1995; Greenwald et al., Bioconiugate Chem., 7: 638-641, 1996; Martinez et al., Macromol. Chem. Phys., 198: 2489-2498, 1997; Hermanson, G. T. , Bioconjugate Techniques, 605-618, 1996; Whitlow et al., Protein Eng., 6: 989-995, 1993; Habeeb, A. F. S. A., Anal. Biochem., 14: 328-336, 1966; Zalipsky et al., Poly(ethyleneglycol) Chemistry and Biological Applications, 318-341, 1997; Harlow et al., Antibodies: a Laboratory Manual, 553-612, Cold Spring Harbor Laboratory, Plainview, N.Y., 1988; Milenic et al, Cancer Res., 51: 6363-6371, 1991; Friguet et al., J. Immunol. Methods, 77: 305-319, 1985, each incorporated herein by reference. While phosphate buffers are commonly employed in these protocols, the choice of borate buffers may beneficially influence the PEGylation reaction rates and resulting products.

It is further contemplated to attach other groups to thio groups of cysteines present in biologically active peptides and proteins for use within the invention. For example, the peptide or protein may be biotinylated by attaching biotin to a thio group of a cysteine residue. Examples are cysteine-PEGylated proteins of the invention, as well as proteins having a group other than PEG covalently attached via a cysteine residue according to the invention.

Other Stabilizing Modifications of Active Agents

In addition to PEGylation, biologically active agents such as peptides and proteins for use within the invention can be modified to enhance circulating half-life by shielding the active agent via conjugation to other known protecting or stabilizing compounds, for example by the creation of fusion proteins with an active peptide, protein, analog or mimetic linked to one or more carrier proteins, such as one or more immunoglobulin chains. U.S. Pat. Nos. 5,750,375; 5,843,725; 5,567,584 and 6,018,026, each incorporated herein by reference. These modifications will decrease the degradation, sequestration or clearance of the active agent and result in a longer half-life in a physiological environment (e.g., in the circulatory system, or at a mucosal surface). The active agents modified by these and other stabilizing conjugations methods are therefore useful with enhanced efficacy within the methods of the invention. In particular, the active agents thus modified maintain activity for greater periods at a target site of delivery or action compared to the unmodified active agent. Even when the active agent is thus modified, it retains substantial biological activity in comparison to a biological activity of the unmodified compound.

In other aspects of the invention, peptide and protein therapeutic compounds are conjugated for enhanced stability with relatively low molecular weight compounds, such as aminolethicin, fatty acids, vitamin B₁₂, and glycosides. Additional exemplary modified peptides and proteins for use within the compositions and methods of the invention will be beneficially modified for in vivo use by:

(a) chemical or recombinant DNA methods to link mammalian signal peptides, Lin et al., J. Biol. Chem., 270: 14255, 1995, or bacterial peptides, Joliot et al., Proc. Natl. Acad. Sci. U.S.A., 88: 1864, 1991, to the active peptide or protein, which serves to direct the active peptide or protein across cytoplasmic and organellar membranes and/or traffic the active peptide or protein to the a desired intracellular compartment (e.g., the endoplasmic reticulum (ER) of antigen presenting cells (APCs), such as dendritic cells for enhanced CTL induction);

(b) addition of a biotin residue to the active peptide or protein which serves to direct the active conjugate across cell membranes by virtue of its ability to bind specifically (i.e., with a binding affinity greater than about 10⁶, 10⁷, 10⁸, 10⁹, or 10¹⁰M⁻¹) to a translocator present on the surface of cells (Chen et al., Analytical Biochem., 227: 168, 1995;

(c) addition at either or both the amino- and carboxy-terminal ends of the active peptide or protein of a blocking agent in order to increase stability in vivo. This can be done either chemically during the synthesis of the peptide or by recombinant DNA technology. Blocking agents such as pyroglutamic acid or other molecules known to those skilled in the art can also be attached to the amino and/or carboxy terminal residues, or the amino group at the amino terminus or carboxyl group at the carboxy terminus can be replaced with a different moiety.

Prodrug Modifications

Yet another processing and formulation strategy useful within the invention is that of prodrug modification. By transiently (i.e., bioreversibly) derivatizing such groups as carboxyl, hydroxyl, and amino groups in small organic molecules, the undesirable physicochemical characteristics (e.g., charge, hydrogen bonding potential, etc. that diminish mucosal penetration) of these molecules can be “masked” without permanently altering the pharmacological properties of the molecule. Bioreversible prodrug derivatives of therapeutic small molecule drugs have been shown to improve the physicochemical (e.g., solubility, lipophilicity) properties of numerous exemplary therapeutics, particularly those that contain hydroxyl and carboxylic acid groups.

One approach to making prodrugs of amine-containing active agents, such as the peptides and proteins of the invention, is through the acylation of the amino group. Optionally, the use of acyloxyalkoxycarbamate derivatives of amines as prodrugs has been discussed. 3-(2′-hydroxy4′,6′-dimethylphenyl)-3,3-dimethylpropionic acid has been employed to prepare linear, esterase-, phosphatase-, and dehydrogenase-sensitive prodrugs of amines (Amsberry et al., Pharm. Res. 8: 455-461, 1991; Wolfe et al., J. Org. Chem. 57: 6138, 1992.

For the purpose of preparing prodrugs of peptides that are useful within the invention, U.S. Pat. No. 5,672,584 (incorporated herein by reference) further describes the preparation and use of cyclic prodrugs of biologically active peptides and peptide nucleic acids (PNAs).

Purification and Preparation

Biologically active agents for mucosal administration according to the invention, for example interferon-α peptides, proteins, analogs and mimetics, and other biologically active agents disclosed herein, are generally provided for direct administration to subjects in a substantially purified form. The term “substantially purified” as used herein, is intended to refer to a peptide, protein, nucleic acid or other compound that is isolated in whole or in part from naturally associated proteins and other contaminants, wherein the peptide, protein, nucleic acid or other active compound is purified to a measurable degree relative to its naturally-occurring state, e.g., relative to its purity within a cell extract.

In certain embodiments, the term “substantially purified” refers to a peptide, protein, or polynucleotide composition that has been isolated from a cell, cell culture medium, or other crude preparation and subjected to fractionation to remove various components of the initial preparation, such as proteins, cellular debris, and other components. Of course, such purified preparations may include materials in covalent association with the active agent, such as glycoside residues or materials admixed or conjugated with the active agent, which may be desired to yield a modified derivative or analog of the active agent or produce a combinatorial therapeutic formulation, conjugate, fusion protein or the like. The term purified thus includes such desired products as peptide and protein analogs or mimetics or other biologically active compounds wherein additional compounds or moieties such as polyethylene glycol, biotin or other moieties are bound to the active agent in order to allow for the attachment of other compounds and/or provide for formulations useful in therapeutic treatment or diagnostic procedures.

Various techniques suitable for use in peptide and protein purification are well known to those of skill in the art. These include, for example, precipitation with ammonium sulfate, PEG, antibodies and the like or by heat denaturation, followed by centrifugation; chromatography steps such as ion exchange, gel filtration, reverse phase, hydroxylapatite and/or affinity chromatography; isoelectric focusing; gel electrophoresis; and combinations of such and other techniques. R. Scopes, Protein Purification: Principles and Practice, Springer-Verlag: New York, 1982, incorporated herein by reference. In general, biologically active peptides and proteins can be extracted from tissues or cell cultures that express the peptides and then immunoprecipitated, where after the peptides and proteins can be further purified by standard protein chemistry/chromatographic methods.

Formulation and Administration

Mucosal delivery formulations of the present invention comprise the biologically active agent to be administered (e.g., one or more of interferon-α(s) and other biologically active agents disclosed herein), typically combined together with one or more pharmaceutically acceptable carriers and, optionally, other therapeutic ingredients. The carrier(s) must be “pharmaceutically acceptable” in the sense of being compatible with the other ingredients of the formulation and not eliciting an unacceptable deleterious effect in the subject. Such carriers are described herein above or are otherwise well known to those skilled in the art of pharmacology. Desirably, the formulation should not include substances such as enzymes or oxidizing agents with which the biologically active agent to be administered is known to be incompatible. The formulations may be prepared by any of the methods well known in the art of pharmacy.

Within the compositions and methods of the invention, interferon-α and other biologically active agents disclosed herein may be administered to subjects by a variety of mucosal administration modes, including by oral, rectal, vaginal, intranasal, intrapulmonary, or transdermal delivery, or by topical delivery to the eyes, ears, skin or other mucosal surfaces. Optionally, interferon-α and other biologically active agents disclosed herein can be coordinately or adjunctively administered by non-mucosal routes, including by intramuscular, subcutaneous, intravenous, intra-atrial, intra-articular, intraperitoneal, or parenteral routes. In other alternative embodiments, the biologically active agent(s) can be administered ex vivo by direct exposure to cells, tissues or organs originating from a mammalian subject, for example as a component of an ex vivo tissue or organ treatment formulation that contains the biologically active agent in a suitable, liquid or solid carrier.

Compositions according to the present invention are often administered in an aqueous solution as a nasal or pulmonary spray and may be dispensed in spray form by a variety of methods known to those skilled in the art. Preferred systems for dispensing liquids as a nasal spray are disclosed in U.S. Pat. No. 4,511,069. Such formulations may be conveniently prepared by dissolving compositions according to the present invention in water to produce an aqueous solution, and rendering said solution sterile. The formulations may be presented in multi-dose containers, for example in the sealed dispensing system disclosed in U.S. Pat. No. 4,511,069.

Other suitable nasal spray delivery systems have been described in Transdermal Systemic Medication, Y. W. Chien Ed., Elsevier Publishers, New York, 1985; and in U.S. Pat. No. 4,778,810. Additional aerosol delivery forms may include, e.g., compressed air-, jet-, ultrasonic-, and piezoelectric nebulizers, which deliver the biologically active agent dissolved or suspended in a pharmaceutical solvent, e.g., water, ethanol, or a mixture thereof.

Nasal and pulmonary spray solutions of the present invention typically comprise the drug or drug to be delivered, optionally formulated with a surface active agent, such as a nonionic surfactant (e.g., polysorbate-80), and one or more buffers. In some embodiments of the present invention, the nasal spray solution further comprises a propellant. The pH of the nasal spray solution is optionally between about pH 6.8 and 7.2, but when desired the pH is adjusted to optimize delivery of a charged macromolecular species (e.g., a therapeutic protein or peptide) in a substantially unionized state. The pharmaceutical solvents employed can also be a slightly acidic aqueous buffer (pH 4-6). Suitable buffers for use within these compositions are as described above or as otherwise known in the art. Other components may be added to enhance or maintain chemical stability, including preservatives, surfactants, dispersants, or gases. Suitable preservatives include, but are not limited to, phenol, methyl paraben, paraben, m-cresol, thiomersal, benzylalkonimum chloride, and the like. Suitable surfactants include, but are not limited to, oleic acid, sorbitan trioleate, polysorbates, lecithin, phosphotidyl cholines, and various long chain diglycerides and phospholipids. Suitable dispersants include, but are not limited to, ethylenediaminetetraacetic acid, and the like. Suitable gases include, but are not limited to, nitrogen, helium, chlorofluorocarbons (CFCs), hydrofluorocarbons (HFCs), carbon dioxide, air, and the like.

Within alternate embodiments, mucosal formulations are administered as dry powder formulations comprising the biologically active agent in a dry, usually lyophilized, form of an appropriate particle size, or within an appropriate particle size range, for intranasal delivery. Minimum particle size appropriate for deposition within the nasal or pulmonary passages is often about 0.5 μ mass median equivalent aerodynamic diameter (MMEAD), commonly about 1 μ MMEAD, and more typically about 2 μ MMEAD. Maximum particle size appropriate for deposition within the nasal passages is often about 10 μ MMEAD, commonly about 8 μ MMEAD, and more typically about 4 μ MMEAD. Intranasally respirable powders within these size ranges can be produced by a variety of conventional techniques, such as jet milling, spray drying, solvent precipitation, supercritical fluid condensation, and the like. These dry powders of appropriate MMEAD can be administered to a patient via a conventional dry powder inhaler (DPI), which rely on the patient's breath, upon pulmonary or nasal inhalation, to disperse the power into an aerosolized amount. Alternatively, the dry powder may be administered via air assisted devices that use an external power source to disperse the powder into an aerosolized amount, e.g., a piston pump.

Dry powder devices typically require a powder mass in the range from about 1 mg to 20 mg to produce a single aerosolized dose (“puff”). If the required or desired dose of the biologically active agent is lower than this amount, the powdered active agent will typically be combined with a pharmaceutical dry bulking powder to provide the required total powder mass. Preferred dry bulking powders include sucrose, lactose, dextrose, mannitol, glycine, trehalose, human serum albumin (HSA), and starch. Other suitable dry bulking powders include cellobiose, dextrans, maltotriose, pectin, sodium citrate, sodium ascorbate, and the like.

To formulate compositions for mucosal delivery within the present invention, the biologically active agent can be combined with various pharmaceutically acceptable additives, as well as a base or carrier for dispersion of the active agent(s). Desired additives include, but are not limited to, pH control agents, such as arginine, sodium hydroxide, glycine, hydrochloric acid, citric acid, etc. In addition, local anesthetics (e.g., benzyl alcohol), isotonizing agents (e.g., sodium chloride, mannitol, sorbitol), adsorption inhibitors (e.g., Tween 80), solubility enhancing agents (e.g., cyclodextrins and derivatives thereof), stabilizers (e.g., serum albumin), and reducing agents (e.g., glutathione) can be included. When the composition for mucosal delivery is a liquid, the tonicity of the formulation, as measured with reference to the tonicity of 0.9% (w/v) physiological saline solution taken as unity, is typically adjusted to a value at which no substantial, irreversible tissue damage will be induced in the nasal mucosa at the site of administration. Generally, the tonicity of the solution is adjusted to a value of about ⅓ to 3, more typically ½ to 2, and most often ¾ to 1.7.

The biologically active agent may be dispersed in a base or vehicle, which may comprise a hydrophilic compound having a capacity to disperse the active agent and any desired additives. The base may be selected from a wide range of suitable carriers, including but not limited to, copolymers of polycarboxylic acids or salts thereof, carboxylic anhydrides (e.g. maleic anhydride) with other monomers (e.g. methyl (meth)acrylate, acrylic acid, etc.), hydrophilic vinyl polymers such as polyvinyl acetate, polyvinyl alcohol, polyvinylpyrrolidone, cellulose derivatives such as hydroxymethylcellulose, hydroxypropylcellulose, etc., and natural polymers such as chitosan, collagen, sodium alginate, gelatin, hyaluronic acid, and nontoxic metal salts thereof. Often, a biodegradable polymer is selected as a base or carrier, for example, polylactic acid, poly(lactic acid-glycolic acid) copolymer, polyhydroxybutyric acid, poly(hydroxybutyric acid-glycolic acid) copolymer and mixtures thereof. Alternatively or additionally, synthetic fatty acid esters such as polyglycerin fatty acid esters, sucrose fatty acid esters, etc. can be employed as carriers. Hydrophilic polymers and other carriers can be used alone or in combination, and enhanced structural integrity can be imparted to the carrier by partial crystallization, ionic bonding, crosslinking and the like. The carrier can be provided in a variety of forms, including, fluid or viscous solutions, gels, pastes, powders, microspheres and films for direct application to the nasal mucosa. The use of a selected carrier in this context may result in promotion of absorption of the biologically active agent.

The biologically active agent can be combined with the base or carrier according to a variety of methods, and release of the active agent may be by diffusion, disintegration of the carrier, or associated formulation of water channels. In some circumstances, the active agent is dispersed in microcapsules (microspheres) or nanocapsules (nanospheres) prepared from a suitable polymer, e.g., isobutyl 2-cyanoacrylate, and dispersed in a biocompatible dispersing medium applied to the nasal mucosa, which yields sustained delivery and biological activity over a protracted time.

To further enhance mucosal delivery of pharmaceutical agents within the invention, formulations comprising the active agent may also contain a hydrophilic low molecular weight compound as a base or excipient. Such hydrophilic low molecular weight compounds provide a passage medium through which a water-soluble active agent, such as a physiologically active peptide or protein, may diffuse through the base to the body surface where the active agent is absorbed. The hydrophilic low molecular weight compound optionally absorbs moisture from the mucosa or the administration atmosphere and dissolves the water-soluble active peptide. The molecular weight of the hydrophilic low molecular weight compound is generally not more than 10000 and preferably not more than 3000. Exemplary hydrophilic low molecular weight compound include polyol compounds, such as oligo-, di- and monosaccarides such as sucrose, mannitol, lactose, L-arabinose, D-erythrose, D-ribose, D-xylose, D-mannose, D-galactose, lactulose, cellobiose, gentibiose, glycerin and polyethylene glycol. Other examples of hydrophilic low molecular weight compounds useful as carriers within the invention include N-methylpyrrolidone, and alcohols (e.g. oligovinyl alcohol, ethanol, ethylene glycol, propylene glycol, etc.) These hydrophilic low molecular weight compounds can be used alone or in combination with one another or with other active or inactive components of the intranasal formulation.

The compositions of the invention may alternatively contain as pharmaceutically acceptable carriers substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc. For solid compositions, conventional nontoxic pharmaceutically acceptable carriers can be used which include, for example, pharmaceutical grades of mannitol, lactose, starch, magnesium stearate, sodium saccharin, talcum, cellulose, glucose, sucrose, magnesium carbonate, and the like.

Therapeutic compositions for administering the biologically active agent can also be formulated as a solution, microemulsion, or other ordered structure suitable for high concentration of active ingredients. The carrier can be a solvent or dispersion medium containing, for example, water, ethanol, polyol (for example, glycerol, propylene glycol, and liquid polyethylene glycol, and the like), and suitable mixtures thereof. Proper fluidity for solutions can be maintained, for example, by the use of a coating such as lecithin, by the maintenance of a desired particle size in the case of dispersible formulations, and by the use of surfactants. In many cases, it will be desirable to include isotonic agents, for example, sugars, polyalcohols such as mannitol, sorbitol, or sodium chloride in the composition. Including in the composition an agent which delays absorption, for example, monostearate salts and gelatin can bring about prolonged absorption of the biologically active agent.

In certain embodiments of the invention, the biologically active agent is administered in a time release formulation, for example in a composition that includes a slow release polymer. The active agent can be prepared with carriers that will protect against rapid release, for example a controlled release vehicle such as a polymer, microencapsulated delivery system or bioadhesive gel. Including in the composition agents that delay absorption, for example, aluminum monosterate hydrogels and gelatin, can bring about prolonged delivery of the active agent, in various compositions of the invention. When controlled release formulations of the biologically active agent is desired, controlled release binders suitable for use in accordance with the invention include any biocompatible controlled-release material which is inert to the active agent and which is capable of incorporating the biologically active agent. Numerous such materials are known in the art. Useful controlled-release binders are materials that are metabolized slowly under physiological conditions following their intranasal delivery (e.g., at the nasal mucosal surface, or in the presence of bodily fluids following transmucosal delivery). Appropriate binders include but are not limited to biocompatible polymers and copolymers previously used in the art in sustained release formulations. Such biocompatible compounds are non-toxic and inert to surrounding tissues, and do not trigger significant adverse side effects such as nasal irritation, immune response, inflammation, or the like. They are metabolized into metabolic products that are also biocompatible and easily eliminated from the body.

Exemplary polymeric materials for use in this context include, but are not limited to, polymeric matrices derived from copolymeric and homopolymeric polyesters having hydrolysable ester linkages. A number of these are known in the art to be biodegradable and to lead to degradation products having no or low toxicity. Exemplary polymers include polyglycolic acids (PGA) and polylactic acids (PLA), poly(DL-lactic acid-co-glycolic acid)(DL PLGA), poly(D-lactic acid-coglycolic acid)(D PLGA) and poly(L-lactic acid-co-glycolic acid)(L PLGA). Other useful biodegradable or bioerodable polymers include but are not limited to such polymers as poly(epsilon-caprolactone), poly(epsilon-aprolactone-CO-lactic acid), poly(ε-aprolactone-CO-glycolic acid), poly(beta-hydroxy butyric acid), poly(alkyl-2- cyanoacrilate), hydrogels such as poly(hydroxyethyl methacrylate), polyamides, poly(amino acids) (i.e., L-leucine, glutamic acid, L-aspartic acid and the like), poly (ester urea), poly (2-hydroxyethyl DL-aspartamide), polyacetal polymers, polyorthoesters, polycarbonate, polymaleamides, polysaccharides and copolymers thereof. Many methods for preparing such formulations are generally known to those skilled in the art (see, e.g., Sustained and Controlled Release Drug Delivery Systems, J. R. Robinson, ed., Marcel Dekker, Inc., New York, 1978,). Other useful formulations include controlled-release compositions such as are known in the art for the administration of leuprolide (trade name: Lupron.RTM.), e.g., microcapsules (U.S. Pat. Nos. 4,652,441 and 4,917,893, each incorporated herein by reference), lactic acid-glycolic acid copolymers useful in making microcapsules and other formulations (U.S. Pat. Nos. 4,677,191 and 4,728,721.

The mucosal formulations of the invention typically must be sterile and stable under all conditions of manufacture, storage and use. Sterile solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders, methods of preparation include vacuum drying and freeze-drying which yields a powder of the active ingredient plus any additional desired ingredient from a previously sterile-filtered solution thereof. The prevention of the action of microorganisms can be accomplished by various antibacterial and antifungal agents, for example, parabens, chlorobutanol, phenol, sorbic acid, thimerosal, and the like.

The term “subject” as used herein means any mammalian patient to which the compositions of the invention may be administered. Typical subjects intended for treatment with the compositions and methods of the present invention include humans, as well as non-human primates and other animals. Mucosal administration according to the invention allows effective self-administration of treatment by patients, provided that sufficient safeguards are in place to control and monitor dosing and side effects. Mucosal administration also overcomes certain drawbacks of other administration forms, such as injections, that are painful and expose the patient to possible infections and may present drug bioavailability problems. For nasal and pulmonary delivery, systems for controlled aerosol dispensing of therapeutic liquids as a spray are well known. In one embodiment, metered doses of active agent are delivered by means of a specially constructed mechanical pump valve (U.S. Pat. No. 4,511,069. This hand-held delivery device is uniquely nonvented so that sterility of the solution in the aerosol container is maintained indefinitely.

Dosage

Determination of effective dosages in this context is typically based on animal model studies followed up by human clinical trials and is guided by determining effective dosages and administration protocols that significantly reduce the occurrence or severity of targeted disease symptoms or conditions in the subject. Suitable models in this regard include, for example, murine, rat, porcine, feline, non-human primate, and other accepted animal model subjects known in the art. Alternatively, effective dosages can be determined using in vitro models (e.g., immunologic and histopathologic assays). Using such models, only ordinary calculations and adjustments are typically required to determine an appropriate concentration and dose to administer a therapeutically effective amount of the biologically active agent(s) (e.g., amounts that are intranasally effective, transdermally effective, intravenously effective, or intramuscularly effective to elicit a desired response). In alternative embodiments, an “effective amount” or “effective dose” of the biologically active agent(s) may simply inhibit or enhance one or more selected biological activity(ies) correlated with a disease or condition, as set forth above, for either therapeutic or diagnostic purposes.

The actual dosage of biologically active agents will of course vary according to factors such as the disease indication and particular status of the subject (e.g., the subject's age, size, fitness, extent of symptoms, susceptibility factors, etc), time and route of administration, other drugs or treatments being administered concurrently, as well as the specific pharmacology of the biologically active agent(s) for eliciting the desired activity or biological response in the subject. Dosage regimens may be adjusted to provide an optimum prophylactic or therapeutic response. A therapeutically effective amount is also one in which any toxic or detrimental side effects of the biologically active agent is outweighed in clinical terms by therapeutically beneficial effects. A non-limiting range for a therapeutically effective amount of a biologically active agent within the methods and formulations of the invention is 0.01 μg/kg-10 mg/kg, more typically between about 0.05 and 5 mg/kg, and in certain embodiments between about 0.2 and 2 mg/kg. Dosages within this range can be achieved by single or multiple administrations, including, e.g., multiple administrations per day, daily or weekly administrations. Per administration, it is desirable to administer at least one microgram of the biologically active agent (e.g., interferon-α and other biologically active agents), more typically between about 10 μg and 5.0 mg, and in certain embodiments between about 100 μg and 1.0 or 2.0 mg to an average human subject. It is to be further noted that for each particular subject, specific dosage regimens should be evaluated and adjusted over time according to the individual need and professional judgment of the person administering or supervising the administration of the permeabilizing peptide(s) and other biologically active agent(s).

The attending clinician to maintain a desired concentration at the target site may vary dosage of biologically active agents. For example, a selected local concentration of the biologically active agent in the bloodstream or CNS may be about 1-50 nanomoles per liter, sometimes between about 1.0 nanomole per liter and 10, 15 or 25 nanomoles per liter, depending on the subject's status and projected or measured response. Higher or lower concentrations may be selected based on the mode of delivery, e.g., trans-epidermal, rectal, oral, or intranasal delivery versus intravenous or subcutaneous delivery. Dosage should also be adjusted based on the release rate of the administered formulation, e.g., of a nasal spray versus powder, sustained release oral versus injected particulate or transdermal delivery formulations, etc. To achieve the same serum concentration level, for example, slow-release particles with a release rate of 5 nanomolar (under standard conditions) would be administered at about twice the dosage of particles with a release rate of 10 nanomolar.

Kits

The instant invention also includes kits, packages and multicontainer units containing the above described pharmaceutical compositions, active ingredients, and/or means for administering the same for use in the prevention and treatment of diseases and other conditions in mammalian subjects. Briefly, these kits include a container or formulation that contains interferon-α and other biologically active agents disclosed herein formulated in a pharmaceutical preparation for mucosal delivery. The biologically active agent(s) is/are optionally contained in a bulk dispensing container or unit or multi-unit dosage form. Optional dispensing means may be provided, for example a pulmonary or intranasal spray applicator. Packaging materials optionally include a label or instruction indicating that the pharmaceutical agent packaged therewith can be used mucosally, e.g., intranasally, for treating or preventing a specific disease or condition.

The following examples are provided by way of illustration, not limitation.

### EXAMPLE 1

Exemplary formulations for enhanced nasal mucosal delivery of interferon-α following the teachings of the instant specification were prepared and evaluated as follows. Tables 1 and 2 indicate dosages for parenteral and mucosal delivery of interferon-α by methods and compositions of the present invention for treatment of disease related to viral infection and tumors in a human subject, for example, hepatitis C, hepatitis B, hairy cell leukemia, HIV infection, AIDS-Kaposi's sarcoma, condylamata acuminata, malignant melanoma, follicular lymphoma.

3 MIU=12 μg Interferon α-2b, recombinant;

5 MIU=19 μg Interferon α-2b, recombinant;

10 MIU=38 μg Interferon α-2b, recombinant;

30 MIU=144 μg Interferon α-2b, recombinant;

60 MIU=288 μg Interferon α-2b, recombinant;

Interferon α-2b, recombinant is, for example, Intron A® (vials 5 MIU; Schering Corp.) Based on the specific activity of approximately 2.6×10⁸ IU/mg protein, as measured by HPLC assay.

TABLE 1
Mucosal delivery of IFN-α as an adjunct with parenteral interferon-α.

| | | | | Nasal |
|---|---|---|---|---|
| | | | Dosages and | Adjunct |
| Drug Name | Route | Indication | Administration | Therapy |
| Interferon α-2a | Parenteral | Hepatitis C | 3 MIU 3X week | Yes |
| | | Hairy Cell Leukemia | for 52 weeks | |
| | | AIDS-Kaposi's Sarcoma | 3 MIU Daily | |
| | | Chronic Myelogenous | for 16-24 weeks | |
| | | Leukemia | 36 MIU Daily | |
| | | | for 10-12 weeks | |
| | | | 9 MIU Daily | |
| Interferon α-2b | Parenteral | Hepatitis C | 3 MIU 3X week | Yes |
| | | Hepatitis B | for 96 weeks | |
| | | Hairy Cell Leukemia | 5 MIU Daily | |
| | | AIDS-Kaposi's Sarcoma | for 16 weeks | |
| | | Condylamata Acuminata | 2 MIU 3X week | |
| | | Malignant Melanoma | for 24 weeks | |
| | | Follicular Lymphoma | 30 MIU 3X week | |
| | | | for 12 weeks | |
| | | | 1.0 MIU 3X week | |
| | | | for 3 weeks | |
| | | | 20 MIU initial IV | |
| | | | 5 days per week | |
| | | | 10 MIU 3X week | |
| | | | for 48 weeks | |
| | | | 5 MIU 3X week | |
| | | | for 72 weeks | |
| Interferon | Parenteral | Hepatitis C | 9 μg 3X | Yes |
| Alfacon | | | for 24 weeks | |
| PEG-Intron | Parenteral | Hepatitis | 1.0 μg/kd 1 a | Yes |
| | | | week | |
| | | | for 24 weeks | |

TABLE 2
Combination therapeutics with mucosal delivery of IFN-α as an adjunct
with parenteral interferon to increase efficacy.

| Indication | Hepatitis B | Hepatitis C | Cancer | HIV |
|---|---|---|---|---|
| Drug | Interferon-α in | Interferon-α in | Interferon-α in | Interferon-α in |
| Combination | combination | combination with | combination with | combination |
| #1 | with | Ribavirin | Radiotherapy | with |
| | Lamivudine | | | Granulocyte |
| | | | | macrophage |
| | | | | colony- |
| | | | | stimulating |
| | | | | factor and |
| | | | | zidovudine |
| Drug | | Interferon-α in | Interferon-α in | |
| Combination | | combination with | combination with | |
| #2 | | Amantadine | chemotherapy | |
| | | | agents | |

### EXAMPLE 2

### Exemplary Pharmaceutical Formulations Comprising Interferon-α-2b and Intranasal Delivery-Enhancing Agents

An exemplary formulation for enhanced nasal mucosal delivery of interferon-α following the teachings of the instant specification was prepared and evaluated as follows. The formulations in Table 3 comprise Intron A® (interferon-α2b; vials 5 MIU; Schering Corp.) in combination with intranasal delivery-enhancing agents of the present invention. The freeze dried powder component of one vial of Intron A® contains 5 MIU interferon-α-2b, 20 mg glycine, 2.3 mg sodium phosphate dibasic, 0.55 mg sodium phosphate monobasic and 1.0 mg human serum albumin. Solutions containing intranasal delivery-enhancing agents were reconstituted by adding 1 mL of solution containing intranasal delivery-enhancing agents to powder content of Intron A® 5MIU/vial.

TABLE 3
Formulations comprising interferon-α-2b and intranasal delivery-
enhancing agents.

| Formulation | Component | Quantity | |
|---|---|---|---|
| 1 | Intron A ® (5 MIU/Vial) | 5 | MIU |
| | N-Caproic Acid Sodium | 1.38 | mg |
| | Purified Water, USP q.s. to | 1 | mL |
| 2 | Intron A ® (5 MIU/Vial) | 5 | MIU |
| | Pluronic —127 | 3 | mg |
| | Purified Water, USP q.s. to | 1 mL | mL |
| 3 | Intron A ® (5 MIU/Vial) | 5 | MIU |
| | Chitosan | 5 | mg |
| | Acetic Acid | QS | |
| | Purified Water, USP q.s. to | 1 mL | mL |
| 4 | Intron A ® (5 MIU/Vial) | 5 | MIU |
| | Alpha-Cyclodextrin | 50 | mg |
| | Purified Water, USP q.s. to | 1 mL | mL |
| 5 | Intron A ® (5 MIU/Vial) | 5 | MIU |
| | Gamma Cyclodextrin | 10 | mg |
| | Purified Water, USP q.s. to | 1 mL | mL |
| 7 | Intron A ® (5 MIU/Vial) | 5 | MIU |
| | Sodium Nitroprusside | 3 | mg |
| | Purified Water, USP q.s. to | 1 mL | mL |
| 8 | Intron A ® (5 MIU/Vial) | 5 | MIU |
| | Sodium Nitroso-N-Acetyl Penecillamine | 5 | mg |
| | Purified Water, USP q.s. to | 1 | mL |
| 10 | Intron A ® (5 MIU/Vial) | 5 | MIU |
| | Arginine | 100 | mg |
| | Purified Water, USP q.s. to | 1 | mL |
| 11 | Intron A ® (5 MIU/Vial) | 5 | MIU |
| | Palmitoyl-DL-Carnitine | 0.003 | mg |
| | Purified Water, USP q.s. to | 1 | mL |
| 11-2 | Intron A ® (5 MIU/Vial) | 5 | MIU |
| | Palmitoyl-DL-Carnitine | 0.2 | mg |
| | Purified Water, USP q.s. to | 1 | mL |
| 12 | Intron A ® (5 MIU/Vial) | 5 | MIU |
| | Didecanyol-1-Alpha- | 25 | mg |
| | Phosphatidylcholine | | |
| | Purified Water, USP q.s. to | 1 | mL |
| 12 | Intron A ® (5 MIU/Vial) | 5 | MIU |
| | poly-GuD | 5.6 | mg |
| | Purified Water, USP q.s. to | 1 | mL |
| 13 | Intron A ® (5 MIU/Vial) | 5 | MIU |
| | Phorbol 12-Myristate-13-Acetate | 6 × 10⁻⁶ | mg |
| | Purified Water, USP q.s. to | 1 | mL |
| 14 | Intron A ® (5 MIU/Vial) | 5 | MIU |
| | EDTA | 5 | mg |
| | Purified Water, USP q.s. to | 1 | mL |
| 15 | Intron A ® (5 MIU/Vial) | 5 | MIU |
| | Sodium Taurocholate | 20 | mg |
| | Purified Water, USP q.s. to | 1 | mL |

### EXAMPLE 3

### Exemplary Pharmaceutical Formulations Comprising Interferon-α-2b and Intranasal Delivery-Enhancing Agents

An exemplary formulation for enhanced nasal mucosal delivery of interferon-αfollowing the teachings of the instant specification was prepared and evaluated as follows. Pharmaceutical formulations in Table 4 comprise Intron A® (interferon alpha 2b; 50 MIU/vial; Schering Corp.), 20 mg glycine, 2.3 mg sodium phosphate dibasic, 0.55 mg sodium phosphate monobasic and 1.0 mg human serum albumin in combination with one or more intranasal delivery-enhancing agents. To prepare the formulation at a concentration of 75 MIU/mL Intron A® (interferon alpha 2b), add 0.67 mL of the solution containing intranasal delivery-enhancing agents to the powder components of Intron A® vial (50 MIU) to a final volume of 1.0 ml of formulation composition. Formulation compositions of the present invention are listed in Table 4.

TABLE 4
Formulations comprising interferon-α-2b and intranasal delivery-
enhancing agents.

| F# | Composition | Quantity | |
|---|---|---|---|
| F1 | Intron A (Interferon-α-2b) | 75 | MIU |
| | Purified Water, USP q.s. to | 1 | mL |
| F2 | Intron A (Interferon-α-2b) | 75 | MIU |
| | Chitosan | 5 | mg |
| | Acetic Acid 1N | 5 | mg |
| | Benzalkonium Chloride 50% | 0.02 | mg |
| | Sodium Deoxycholate | 1 | mg |
| | Methyl-b-Cyclodextrin | 50 | mg |
| | EDTA | 0.005 | mg |
| | Sodium Hydroxide | QS | |
| | Purified Water, USP q.s. to | 1 | mL |
| F2-R | Intron A (Interferon-α-2b) | 75 | MIU |
| | Poly (Gud) | 5 | mg |
| | Acetic Acid 1N | 5 | mg |
| | Benzalkonium Chloride 50% | 0.02 | mg |
| | Sodium Deoxycholate | 1 | mg |
| | Methyl-β-Cyclodextrin | 50 | mg |
| | EDTA | 0.005 | mg |
| | Sodium Hydroxide | QS | |
| | Purified Water, USP q.s. to | 1 | mL |
| F3 | Intron A (Interferon-α-2b) | 75 | MIU |
| | Chitosan | 5 | mg |
| | Acetic Acid | 5 | mg |
| | Benzalkonium Chloride 50% | 0.02 | mg |
| | Sodium Deoxycholate | 1 | mg |
| | Sodium Hydroxide | QS | |
| | Purified Water, USP q.s. to | 1 | mL |
| F4 | Intron A (Interferon-α-2b) | 75 | MIU |
| | Chitosan | 5 | mg |
| | Acetic Acid | 5 | mg |
| | Benzalkonium Chloride 50% | 0.02 | mg |
| | Methyl-Beta-Cyclodextrin | 50 | mg |
| | EDTA | 0.005 | mg |
| | Sodium Hydroxide | QS | |
| | Purified Water, USP q.s. to | 1 | mL |
| F5 | Intron A (Interferon-α-2b) | 75 | MIU |
| | Carbopol 934P | 0.5 | mg |
| | Sodium Hydroxide | QS | |
| | Bacetracin | 0.1 | mg |
| | Benzalkonium Chloride 50% | 0.02 | mg |
| | Oleic Acid | 1 | mg |
| | Tragacanth | 0.1 | mg |
| | Purified Water, USP q.s. to | 1 | mL |
| F6 | Intron A (Interferon-α-2b) | 75 | MIU |
| | Carbopol 934P | 0.5 | mg |
| | Sodium Hydroxide | QS | |
| | Bacitracin | 0.1 | mg |
| | Benzyl Alcohol | 1 | mg |
| | EDTA | 0.1 | mg |
| | PEG 300 | 2 | mg |
| | Purified Water, USP q.s. to | 1 | mL |
| F8 | Intron A (Interferon-α-2b) | 75 | MIU |
| | Carbopol 934P | 0.5 | mg |
| | Sodium Hydroxide | QS | |
| | Sodium Deoxycholate | 0.1 | mg |
| | Benzalkonium Chloride 50% | 0.002 | mg |
| | Purified Water, USP q.s. to | 1 | mL |
| F9 | Intron A (Interferon-α-2b) | 75 | MIU |
| | Benzyl Alcohol | 10 | mg |
| | Oleic Acid | 2 | mg |
| | Pectin | 1 | mg |
| | Sodium Hydroxide | QS | |
| | Hydrochloric Acid | QS | |
| | Purified Water, USP q.s. to | 1 | mL |
| F10 | Intron A (Interferon-α-2b) | 75 | MIU |
| | HPMC (4000 cps) | 2 | mg |
| | Sodium Taurodeoxycholate | 2 | mg |
| | Benzyl Alcohol | 10 | mg |
| | Lisophosphatidyl Choline | 0.01 | mg |
| | Purified Water, USP q.s. to | 1 | mL |
| F13 | Intron A (Interferon-α-2b) | 75 | MIU |
| | Poly-L-Arginine | 1 | mg |
| | Sodium-Nitroso-N-Acetyl | 0.44 | mg |
| | Penecillamine | | |
| | Sodium n-Caproic Acid | 1.38 | mg |
| | Benzalkonium Chloride 50% | 0.02 | mg |
| | HPMC (4000 cps) | 2 | mg |
| | Purified Water, USP q.s. to | 1 | mL |
| F14 | PEG-Intron A (Interferon-α-2b) | 75 | MIU |
| | Dibasic sodium phosphate anhydrous | 1.11 | mg |
| | Monobasic sodium phosphate dihydrate | 1.11 | mg |
| | Sucrose | 59.2 | mg |
| | Polysorbate 80 (Stabilizer) | 0.074 | mg |
| | Benzalkonium Chloride 50% | 2.0 | mg |
| | L-Alpha-phosphatidylcholine Didecanyl | 5.0 | mg |
| | Methyl Beta Cyclodextrin | 30.0 | mg |
| | EDTA | 1.0 | mg |
| | Gelatin | 5.0 | mg |
| | Purified Water, USP q.s. to | 1.0 | mL |

### EXAMPLE 4

### Mucosal Delivery-Permeation Kinetics and Cytotoxicity

1. Organotypic Model

The following methods are generally useful for evaluating mucosal delivery parameters, kinetics and side effects for IFN-α within the formulations and method of the invention, as well as for determining the efficacy and characteristics of the various mucosal delivery-enhancing agents disclosed herein for combinatorial formulation or coordinate administration with IFN-α.

Permeation kinetics and cytotoxicity are also useful for determining the efficacy and characteristics of the various mucosal delivery-enhancing agents disclosed herein for combinatorial formulation or coordinate administration with mucosal delivery-enhancing agents. In one exemplary protocol, permeation kinetics and lack of unacceptable cytotoxicity are demonstrated for an intranasal delivery-enhancing agents as disclosed above in combination with a biologically active therapeutic agent, exemplified by interferon-α.

The EpiAirway system was developed by MatTek Corp (Ashland, Mass.) as a model of the pseudostratified epithelium lining the respiratory tract. The epithelial cells are grown on porous membrane-bottomed cell culture inserts at an air-liquid interface, which results in differentiation of the cells to a highly polarized morphology. The apical surface is ciliated with a microvillous ultrastructure and the epithelium produces mucus (the presence of mucin has been confirmed by immunoblotting). The inserts have a diameter of 0.875 cm, providing a surface area of 0.6 cm². The cells are plated onto the inserts at the factory approximately three weeks before shipping. One “kit” consists of 24 units.

A. On arrival, the units are placed onto sterile supports in 6-well microplates. Each well receives 5 mL of proprietary culture medium. This DMEM-based medium is serum free but is supplemented with epidermal growth factor and other factors. The medium is always tested for endogenous levels of any cytokine or growth factor which is being considered for intranasal delivery, but has been free of all cytokines and factors studied to date except insulin. The 5 mL volume is just sufficient to provide contact to the bottoms of the units on their stands, but the apical surface of the epithelium is allowed to remain in direct contact with air. Sterile tweezers are used in this step and in all subsequent steps involving transfer of units to liquid-containing wells to ensure that no air is trapped between the bottoms of the units and the medium.

B. The units in their plates are maintained at 37° C. in an incubator in an atmosphere of 5% CO₂ in air for 24 hours. At the end of this time the medium is replaced with fresh medium and the units are returned to the incubator for another 24 hours.

2. Experimental Protocol—Permeation Kinetics

A. A “kit” of 24 EpiAirway units can routinely be employed for evaluating five different formulations, each of which is applied to quadruplicate wells. Each well is employed for determination of permeation kinetics (4 time points), transepithelial resistance, mitochondrial reductase activity as measured by MTT reduction, and cytolysis as measured by release of LDH. An additional set of wells is employed as controls, which are sham treated during determination of permeation kinetics, but are otherwise handled identically to the test sample-containing units for determinations of transepithelial resistance and viability. The determinations on the controls are routinely also made on quadruplicate units, but occasionally we have employed triplicate units for the controls and have dedicated the remaining four units in the kit to measurements of transepithelial resistance and viability on untreated units or we have frozen and thawed the units for determinations of total LDH levels to serve as a reference for 100% cytolysis.

B. In all experiments, the mucosal delivery formulation to be studied is applied to the apical surface of each unit in a volume of 100 μL, which is sufficient to cover the entire apical surface. An appropriate volume of the test formulation at the concentration applied to the apical surface (no more than 100 μL is generally needed) is set aside for subsequent determination of concentration of the active material by ELISA or other designated assay.

C. The units are placed in 6 well plates without stands for the experiment: each well contains 0.9 mL of medium which is sufficient to contact the porous membrane bottom of the unit but does not generate any significant upward hydrostatic pressure on the unit.

D. In order to minimize potential sources of error and avoid any formation of concentration gradients, the units are transferred from one 0.9 mL-containing well to another at each time point in the study. These transfers are made at the following time points, based on a zero time at which the 100 αL volume of test material was applied to the apical surface: 15 minutes, 30 minutes, 60 minutes, and 120 minutes.

E. In between time points the units in their plates are kept in the 37° C. incubator. Plates containing 0.9 mL medium per well are also maintained in the incubator so that minimal change in temperature occurs during the brief periods when the plates are removed and the units are transferred from one well to another using sterile forceps.

F. At the completion of each time point, the medium is removed from the well from which each unit was transferred, and aliquotted into two tubes (one tube receives 700 μL and the other 200 μL) for determination of the concentration of permeated test material and, in the event that the test material is cytotoxic, for release of the cytosolic enzyme, lactic dehydrogenase, from the epithelium. These samples are kept in the refrigerator if the assays are to be conducted within 24 hours, or the samples are subaliquotted and kept frozen at −80° C. until thawed once for assays. Repeated freeze-thaw cycles are to be avoided.

G. In order to minimize errors, all tubes, plates, and wells are prelabeled before initiating an experiment.

H. At the end of the 120 minute time point, the units are transferred from the last of the 0.9 mL containing wells to 24-well microplates, containing 0.3 mL medium per well. This volume is again sufficient to contact the bottoms of the units, but not to exert upward hydrostatic pressure on the units. The units are returned to the incubator prior to measurement of transepithelial resistance.

3. Experimental Protocol—Transepithelial Resistance

A. Respiratory airway epithelial cells form tight junctions in vivo as well as in vitro, restricting the flow of solutes across the tissue. These junctions confer a transepithelial resistance of several hundred ohms×cm² in excised airway tissues; in the MatTek EpiAirway units, the transepithelial resistance (TER) is claimed by the manufacturer to be routinely around 1000 ohms×cm². We have found that the TER of control EpiAirway units which have been sham-exposed during the sequence of steps in the permeation study is somewhat lower (700-800 ohms×cm²), but, since permeation of small molecules is proportional to the inverse of the TER, this value is still sufficiently high to provide a major barrier to permeation. The porous membrane-bottomed units without cells, conversely, provide only minimal transmembrane resistance (5-20 ohms×cm²).

B. Accurate determinations of TER require that the electrodes of the ohmmeter be positioned over a significant surface area above and below the membrane, and that the distance of the electrodes from the membrane be reproducibly controlled. The method for TER determination recommended by MatTek and employed for all experiments here employs an “EVOM”™ epithelial voltohmmeter and an “ENDOHM”™ tissue resistance measurement chamber from World Precision Instruments, Inc., Sarasota, Fla.

C. The chamber is initially filled with Dulbecco's phosphate buffered saline (PBS) for at least 20 minutes prior to TER determinations in order to equilibrate the electrodes.

D. Determinations of TER are made with 1.5 mL of PBS in the chamber and 350 μL of PBS in the membrane-bottomed unit being measured. The top electrode is adjusted to a position just above the membrane of a unit containing no cells (but containing 350 μL of PBS) and then fixed to ensure reproducible positioning. The resistance of a cell-free unit is typically 5-20 ohms×cm² (“background resistance”).

E. Once the chamber is prepared and the background resistance is recorded, units in a 24-well plate which had just been employed in permeation determinations are removed from the incubator and individually placed in the chamber for TER determinations.

F. Each unit is first transferred to a petri dish containing PBS to ensure that the membrane bottom is moistened. An aliquot of 350 μL PBS is added to the unit and then carefully aspirated into a labeled tube to rinse the apical surface. A second wash of 350 μL PBS is then applied to the unit and aspirated into the same collection tube.

G. The unit is gently blotted free of excess PBS on its exterior surface only before being placed into the chamber (containing a fresh 1.5 mL aliquot of PBS). An aliquot of 350 μL PBS is added to the unit before the top electrode is placed on the chamber and the TER is read on the EVOM meter.

H. After the TER of the unit is read in the ENDOHM chamber, the unit is removed, the PBS is aspirated and saved, and the unit is returned with an air interface on the apical surface to a 24-well plate containing 0.3 mL medium per well.

I. The units are read in the following sequence: all sham-treated controls, followed by all formulation-treated samples, followed by a second TER reading of each of the sham-treated controls. After all the TER determinations are complete, the units in the 24-well microplate are returned to the incubator for determination of viability by MTT reduction.

4. Experimental Protocol—Viability by MTT Reduction

MTT is a cell-permeable tetrazolium salt which is reduced by mitochondrial dehydrogenase activity to an insoluble colored formazan by viable cells with intact mitochondrial function or by nonmitochondrial NAD(P)H dehydrogenase activity from cells capable of generating a respiratory burst. Formation of formazan is a good indicator of viability of epithelial cells since these cells do not generate a significant respiratory burst. We have employed a MTT reagent kit prepared by MatTek Corp for their units in order to assess viability.

A. The MTT reagent is supplied as a concentrate and is diluted into a proprietary DMEM-based diluent on the day viability is to be assayed (typically the afternoon of the day in which permeation kinetics and TER were determined in the morning). Insoluble reagent is removed by a brief centrifugation before use. The final MTT concentration is 1 mg/mL

B. The final MTT solution is added to wells of a 24-well microplate at a volume of 300 μL per well. As has been noted above, this volume is sufficient to contact the membranes of the EpiAirway units but imposes no significant positive hydrostatic pressure on the cells.

C. The units are removed from the 24-well plate in which they were placed after TER measurements, and after removing any excess liquid from the exterior surface of the units, they are transferred to the plate containing MTT reagent. The units in the plate are then placed in an incubator at 37° C. in an atmosphere of 5% CO₂ in air for 3 hours.

D. At the end of the 3-hour incubation, the units containing viable cells will have turned visibly purple. The insoluble formazan must be extracted from the cells in their units to quantitate the extent of MTT reduction. Extraction of the formazan is accomplished by transferring the units to a 24-well microplate containing 2 mL extractant solution per well, after removing excess liquid from the exterior surface of the units as before . This volume is sufficient to completely cover both the membrane and the apical surface of the units. Extraction is allowed to proceed overnight at room temperature in a light-tight chamber. MTT extractants traditionally contain high concentrations of detergent, and destroy the cells.

E. At the end of the extraction, the fluid from within each unit and the fluid in its surrounding well are combined and transferred to a tube for subsequent aliquotting into a 96-well microplate (200 μL aliquots are optimal) and determination of absorbance at 570 nm on a VMax multiwell microplate spectrophotometer. To ensure that turbidity from debris coming from the extracted units does not contribute to the absorbance, the absorbance at 650 nm is also determined for each well in the VMax and is automatically subtracted from the absorbance at 570 nm. The “blank” for the determination of formazan absorbance is a 200 μL aliquot of extractant to which no unit had been exposed. This absorbance value is assumed to constitute zero viability.

F. Two units from each kit of 24 EpiAirway units are left untreated during determination of permeation kinetics and TER. These units are employed as the positive control for 100% cell viability. In all the studies we have conducted, there has been no statistically significant difference in the viability of the cells in these untreated units vs cells in control units which had been sham treated for permeation kinetics and on which TER determinations had been performed. The absorbance of all units treated with test formulations is assumed to be linearly proportional to the percent viability of the cells in the units at the time of the incubation with MTT. It should be noted that this assay is carried out typically no sooner than four hours after introduction of the test material to the apical surface, and subsequent to rinsing of the apical surface of the units during TER determination.

5. Determination of Viability by LDH Release

While measurement of mitochondrial reductase activity by MTT reduction is a sensitive probe of cell viability, the assay necessarily destroys the cells and therefore can be carried out only at the end of each study. When cells undergo necrotic lysis, their cytotosolic contents are spilled into the surrounding medium, and cytosolic enzymes such as lactic dehydrogenase (LDH) can be detected in this medium. An assay for LDH in the medium can be performed on samples of medium removed at each time point of the two-hour determination of permeation kinetics. Thus, cytotoxic effects of formulations which do not develop until significant time has passed can be detected as well as effects of formulations which induce cytolysis with the first few minutes of exposure to airway epithelium.

A. The recommended LDH assay for evaluating cytolysis of the EpiAirway units is based on conversion of lactate to pyruvate with generation of NADH from NAD. The NADH is then reoxidized along with simultaneous reduction of the tetrazolium salt INT, catalyzed by a crude “diaphorase” preparation. The formazan formed from reduction of INT is soluble, so that the entire assay for LDH activity can be carried out in a homogenous aqueous medium containing lactate, NAD, diaphorase, and INT.

B. The assay for LDH activity is carried out on 50 μL aliquots from samples of “supernatant” medium surrounding an EpiAirway unit and collected at each time point. These samples were either stored for no longer than 24 h in the refrigerator or were thawed after being frozen within a few hours after collection. Each EpiAirway unit generates samples of supernatant medium collected at 15 min, 30 min, 1 h, and 2 h after application of the test material. The aliquots are all transferred to a 96 well microplate.

C. A 50 μL aliquot of medium which had not been exposed to a unit serves as a “blank” or negative control of 0% cytotoxicity. We have found that the apparent level of “endogenous” LDH present after reaction of the assay reagent mixture with the unexposed medium is the same within experimental error as the apparent level of LDH released by all the sham-treated control units over the entire time course of 2 hours required to conduct a permeation kinetics study. Thus, within experimental error, these sham-treated units show no cytolysis of the epithelial cells over the time course of the permeation kinetics measurements.

D. To prepare a sample of supernatant medium reflecting the level of LDH released after 100% of the cells in a unit have lysed, a unit which had not been subjected to any prior manipulations is added to a well of a 6-well microplate containing 0.9 mL of medium as in the protocol for determination of permeation kinetics, the plate containing the unit is frozen at −80° C., and the contents of the well are then allowed to thaw. This freeze-thaw cycle effectively lyses the cells and releases their cytosolic contents, including LDH, into the supernatant medium. A 50 μL aliquot of the medium from the frozen and thawed cells is added to the 96-well plate as a positive control reflecting 100% cytotoxicity.

E. To each well containing an aliquot of supernatant medium, a 50 μL aliquot of the LDH assay reagent is added. The plate is then incubated for 30 minutes in the dark.

F. The reactions are terminated by addition of a “stop” solution of 1 M acetic acid, and within one hour of addition of the stop solution, the absorbance of the plate is determined at 490 nm.

G. Computation of percent cytolysis is based on the assumption of a linear relationship between absorbance and cytolysis, with the absorbance obtained from the medium alone serving as a reference for 0% cytolysis and the absorbance obtained from the medium surrounding a frozen and thawed unit serving as a reference for 100% cytolysis.

6. ELISA Determinations

The procedures for determining the concentrations of biologically active agents as test materials for evaluating enhanced permeation of active agents in conjunction with coordinate administration of mucosal delivery-enhancing agents or combinatorial formulation of the invention are generally as described above and in accordance with known methods and specific manufacturer instructions of ELISA kits employed for each particular assay. Permeation kinetics of the biologically active agent is generally determined by taking measurements at multiple time points (for example 15 min., 30 min., 60 min. and 120 min) after the biologically active agent is contacted with the apical epithelial cell surface (which may be simultaneous with, or subsequent to, exposure of the apical cell surface to the mucosal delivery-enhancing agent(s)).

EpiAirway™ tissue membranes are cultured in phenol red and hydrocortisone free medium (MatTek Corp., Ashland, Mass.). The tissue membranes are cultured at 37° C. for 48 hours to allow the tissues to equilibrate. Each tissue membrane is placed in an individual well of a 6-well plate containing 0.9 mL of serum free medium. 100 μL of the formulation (test sample or control) is applied to the apical surface of the membrane. Triplicate or quadruplicate samples of each test sample (mucosal delivery-enhancing agent in combination with a biologically active agent, interferon-β) and control (biologically active agent, interferon-β, alone) are evaluated in each assay. At each time point (15, 30, 60 and 120 minutes) the tissue membranes are moved to new wells containing fresh medium. The underlying 0.9 mL medium samples is harvested at each time point and stored at 4° C. for use in ELISA and lactate dehydrogenase (LDH) assays.

The ELISA kits are typically two-step sandwich ELISAs: the immunoreactive form of the agent being studied is first “captured” by an antibody immobilized on a 96-well microplate and after washing unbound material out of the wells, a “detection” antibody is allowed to react with the bound immunoreactive agent. This detection antibody is typically conjugated to an enzyme (most often horseradish peroxidase) and the amount of enzyme bound to the plate in immune complexes is then measured by assaying its activity with a chromogenic reagent. In addition to samples of supernatant medium collected at each of the time points in the permeation kinetics studies, appropriately diluted samples of the formulation (i.e., containing the subject biologically active test agent) that was applied to the apical surface of the units at the start of the kinetics study are also assayed in the ELISA plate, along with a set of manufacturer-provided standards. Each supernatant medium sample is generally assayed in duplicate wells by ELISA (it will be recalled that quadruplicate units are employed for each formulation in a permeation kinetics determination, generating a total of sixteen samples of supernatant medium collected over all four time points).

A. It is not uncommon for the apparent concentrations of active test agent in samples of supernatant medium or in diluted samples of material applied to the apical surface of the units to lie outside the range of concentrations of the standards after completion of an ELISA. No concentrations of material present in experimental samples are determined by extrapolation beyond the concentrations of the standards; rather, samples are rediluted appropriately to generate concentrations of the test material which can be more accurately determined by interpolation between the standards in a repeat ELISA.

B. The ELISA for a biologically active test agent, for example, interferon-β, is unique in its design and recommended protocol. Unlike most kits, the ELISA employs two monoclonal antibodies, one for capture and another, directed towards a nonoverlapping determinant for the biologically active test agent, e.g., interferon-β, as the detection antibody (this antibody is conjugated to horseradish peroxidase). As long as concentrations of IFN-α that lie below the upper limit of the assay are present in experimental samples, the assay protocol can be employed as per the manufacturer's instructions, which allow for incubation of the samples on the ELISA plate with both antibodies present simultaneously. When the IFN-α levels in a sample are significantly higher than this upper limit, the levels of immunoreactive IFN-α may exceed the amounts of the antibodies in the incubation mixture, and some IFN-αwhich has no detection antibody bound will be captured on the plate, while some IFN-α which has detection antibody bound may not be captured. This leads to serious underestimation of the IFN-α levels in the sample (it will appear that the IFN-α levels in such a sample lie significantly below the upper limit of the assay). To eliminate this possibility, the assay protocol has been modified:

B.1. The diluted samples are first incubated on the ELISA plate containing the immobilized capture antibody for one hour in the absence of any detection antibody. After the one hour incubation, the wells are washed free of unbound material.

B.2. The detection antibody is incubated with the plate for one hour to permit formation of immune complexes with all captured antigen. The concentration of detection antibody is sufficient to react with the maximum level of IFN-α which has been bound by the capture antibody. The plate is then washed again to remove any unbound detection antibody.

B.3. The peroxidase substrate is added to the plate and incubated for fifteen minutes to allow color development to take place.

B.4. The “stop” solution is added to the plate, and the absorbance is read at 450 nm as well as 490 nm in the VMax microplate spectrophotometer. The absorbance of the colored product at 490 nm is much lower than that at 450 nm, but the absorbance at each wavelength is still proportional to concentration of product. The two readings ensure that the absorbance is linearly related to the amount of bound IFN-α over the working range of the VMax instrument (we routinely restrict the range from 0 to 2.5 OD, although the instrument is reported to be accurate over a range from 0 to 3.0 OD). The amount of IFN-α in the samples is determined by interpolation between the OD values obtained for the different standards included in the ELISA. Samples with OD readings outside the range obtained for the standards are rediluted and run in a repeat ELISA.

### RESULTS

Measurement of Transepithelial Resistance by TER Assay:

After the final assay time points, membranes were placed in individual wells of a 24 well culture plate in 0.3 mL of clean medium and the transepithelial electrical resistance (TER) was measured using the EVOM Epithelial Voltohmmeter and an Endohm chamber (World Precision Instruments, Sarasota, Fla.). The top electrode was adjusted to be close to, but not in contact with, the top surface of the membrane. Tissues were removed, one at a time, from their respective wells and basal surfaces were rinsed by dipping in clean PBS. Apical surfaces were gently rinsed twice with PBS. The tissue unit was placed in the Endohm chamber, 250 μL of PBS added to the insert, the top electrode replaced and the resistance measured and recorded. Following measurement, the PBS was decanted and the tissue insert was returned to the culture plate. All TER values are reported as a function of the surface area of the tissue.

The final numbers were calculated as:

TER of cell membrane=(Resistance (R) of Insert with membrane−R of blank Insert)×Area of membrane (0.6 cm²).

The effect of pharmaceutical formulations comprising interferon-α and intranasal delivery-enhancing agents on TER measurements across the EpiAirway™ Cell Membrane (mucosal epithelial cell layer) is shown in Tables 5, 6, and 7. A decrease in TER value relative to the control value (control=approximately 1000 ohms-cm² ; normalized to 100.) indicates a decrease in cell membrane resistance and an increase in mucosal epithelial cell permeability.

Exemplary formulations for enhanced intranasal delivery of interferon-α decrease cell membrane resistance and increase permeability of mucosal epithelial cells by in vitro TER assay. Stable pharmaceutical formulations comprising an intranasal effective amount of interferon-α and one or more intranasal delivery-enhancing agents are indicated in Table 5. An increase in permeability of mucosal epithelial cells occurred in exemplary formulations containing intranasal delivery-enhancing agents, for example, sodium turocholate (2% w/v), poly-GuD (0.56% w/v, pH=4.2), chitosan (0.5% w/v), or EDTA disodium (0.5% w/v).

Exemplary formulations F-2, F-2-R, F-9, F-3, F-13. , and F-8 showed the greatest decrease in cell membrane resistance by TER assay indicating an increase in mucosal epithelial cell permeability. The results of TER measurements of the mucosal epithelial cell layer treated are shown in Table 6.

PEG-interferon-α (31 kD) is a high molecular weight form of IFN-α that is useful in a sustained release formulation. Exemplary Formulation F-14 (comprising PEG-interferon-α and intranasal delivery-enhancing agents of the present invention) showed an increase in mucosal epithelial cell permeability as measured by the TER assay that is 49-fold higher than PEG-interferon-α alone See Table 7. Exemplary Formulation F-14 showed an increase in mucosal epithelial cell permeability as measured by ELISA assay that is 56-fold higher than PEG-interferon-α alone See Table 9. Formulation F-14 shows no significant cytotoxicity by LDH assay or MTT assay See Table 11.

The exemplary formulations demonstrate enhanced intranasal delivery of interferon-α to the blood serum or central nervous system. The results indicate that these exemplary formulations when contacted with a mucosal epithelium yield significant increases in mucosal epithelial cell permeability to interferon-α.

TABLE 5
Influence of Pharmaceutical Formulations Comprising Interferon-
α-2b and Intranasal Delivery-Enhancing Agents on TER of
EpiAirway ™ Cell Membrane

| Pharmaceutical | % Permeated |
|---|---|
| Formulations | by TER Assay |
| No Treatment (Control-1) | 100 |
| N-Caproic Acid Sodium (0.138% w/v) | 88.83 |
| Pluronic 127 (0.3% w/v) | 90.99 |
| Chitosan (0.5% w/v) | 15.65 |
| Alpha-Cyclodextrin (5% w/v) | 36.19 |
| Gamma Cyclodextrin (1% w/v) | 90.98 |
| No Treatment (Control-2) | 100 |
| Sodium Nitroprusside, 0.3% | 67.43 |
| SNAP (0.5 W/V %) | 69.64 |
| Arginine (10% w/v) | 34.46 |
| Palmitoyl-DL-Carnitine (0.0003% w/v) | 91.78 |
| No Treatment (Control-3) | 100 |
| Palmitoyl-DL-Carnitine (0.02% w/v) | 100.56 |
| Didecanyol-l-Alpha-Phosphatidylcholine | 6.82 |
| (2.5% w/v) | |
| No Treatment (Control-4) | 100 |
| Chitosan (0.5% w/v)-2 | 7.94 |
| Poly-GuD 0.56% (w/v)-pH = 6.4 | 50.36 |
| No Treatment (Control-4) | 100 |
| Chitosan (0.5% w/v)-3 | 4.49 |
| EDTA (0.5% w/v) | 5.75 |
| Phorpol 6 × 10⁻⁶) | 100 |
| Sodium Turocholate (2.0 w/v) | 10.63 |
| Poly-GuD 0.56% (w/v)-pH = 4.2 | 6.98 |

TABLE 6
Influence of Pharmaceutical Formulations Comprising Interferon-
α-2a and Intranasal Delivery-Enhancing Agents on TER of
EpiAirway Cell Membrane

| Pharmaceutical | |
|---|---|
| Formulation | % TER |
| No treatment | 100 |
| F1 Control (Intron-A; IFN-α-2b) | 100 |
| F5 (Intron-A; IFN-α-2b, Carbopol 934P, Bacitracin, Oleic) | 100 |
| F6 (Intron-A; IFN-α-2b, Carbopol 934P, Bacitracin, EDTA) | 93.6 |
| F4 (Intron-A; IFN-α-2b, Chitosan, MBCD, EDTA) | 49.6 |
| F10 (Intron-A; IFN-α-2b, HPMC, STC, LPDC) | 22.3 |
| F2 R (Intron-A; IFN-α-2b, Poly(Gud), SDC, MBCD, EDTA) | 15.1 |
| F9 (Intron-A; IFN-α-2b, Oleic, Pectin) | 13.2 |
| F3 (Intron-A; IFN-α-2b, Chitosan, SDC) | 6.3 |
| F13 (Intron-A; IFN-α-2b, HPMC, Poly-L-Arginine, SNAP, | 8.3 |
| SCA) | |
| F8 (Intron-A; IFN-α-2b, Carbopol 934P, SDC) | 4.9 |
| F2 (Intron-A; IFN-α-2b, Chitosan, SDC, MBCD, EDTA) | 0.9 |

TABLE 7
Influence of Pharmaceutical Formulations Comprising PEG-
Interferon-α-2a and Intranasal Delivery-Enhancing Agents on
TER of EpiAirway Cell Membrane

| Pharmaceutical | |
|---|---|
| Formulation | % TER |
| No Treatment | 100 |
| Control (PEG-Intron A; IFN-α-2b) | 88 |
| F14 (PEG-Intron-A; IFN-α-2b, BAC, DDPC, MBCD, EDTA) | 1.8 |

Permeation Kinetics as Measured by ELISA Assay:

The effect of pharmaceutical formulations comprising interferon-α-2b and intranasal delivery-enhancing agents on the permeation of interferon-α-2b across the EpiAirway™ Cell Membrane (mucosal epithelial cell layer) is measured as described above. The results are shown in Tables 8 and 9. Permeation of interferon-α-2b across the EpiAirway™ Cell Membrane is measured by ELISA assay.

For the exemplary intranasal formulations of the present invention, the greatest increase in interferon-α-2b permeation occurred in Formulation F-2 (135-fold increase in premeation) and Formulation F-2-R (37-fold increase in permeation) compared to Intron A formulation control. See Table 9. Further exemplary formulations are Formulations F-8, F-3, F-10 and F-4 showing increased mucosal epithelial cell permeability of 34-fold, 25-fold, 25-fold, and 3-fold, respectively compared to Intron A formulation control. Further exemplary formulations comprising didecanyol-1-α-phosphatidylcholine, chitosan, poly-GuD, EDTA, or sodium turocholate show increased mucosal epithelial cell permeability compared to Intron A formulation control. See Table 8. Exemplary Formulation F-14 showed an increase in mucosal epithelial cell permeability as measured by ELISA assay that is 56-fold higher than PEG-interferon-α alone See Table 9.

TABLE 8
Influence of Pharmaceutical Formulations Comprising Interferon-
α-2b and Intranasal Delivery-Enhancing Agents on Permeation of
Interferon-α-2b through EpiAirway Cell Membrane as Measured by
ELISA Assay

| | % Permeated | | | | |
|---|---|---|---|---|---|
| Pharmaceutical | Time | Time | Time | Time | |
| Formulations | 0 min | 15 min | 30 min | 60 min | 120 min |
| N-Caproic Acid Sodium | 0.0000 | 0.0000 | 0.0001 | 0.0005 | 0.0051 |
| (0.138% w/v) | | | | | |
| Pluronic-127 (0.3% w/v) | 0.0000 | 0.0000 | 0.0000 | 0.0003 | 0.0039 |
| Chitosan (0.5% w/v) | 0.0000 | 0.0000 | 0.0010 | 0.0340 | 0.2447 |
| Alpha - Cyclodextrin | 0.0000 | 0.0012 | 0.0364 | 0.0813 | 0.1804 |
| (5% w/v) | | | | | |
| Gamma Cyclodexrin | 0.0000 | 0.0000 | 0.0000 | 0.0002 | 0.0025 |
| (1% w/v) | | | | | |
| Sodium Nitroprusside, | 0.0000 | 0.0000 | 0.0001 | 0.0001 | 0.0007 |
| 0.3% | | | | | |
| SNAP (0.5% w/v) | 0.00000 | 0.00072 | 0.00096 | 0.00097 | 0.00102 |
| Arginine (10% w/v) | 0.0000 | 0.0124 | 0.0550 | 0.1275 | 0.2200 |
| Palmitoyl-DL-Carnitine | 0.0000 | 0.0008 | 0.0008 | 0.0011 | 0.0049 |
| (0.0003% w/v) | | | | | |
| Palmitoyl-DL-Carnitine | 0.00000 | 0.00007 | 0.00009 | 0.00014 | 0.00021 |
| (0.02% w/v) | | | | | |
| Didecanyol-1-Alpha | 0.0000 | 0.0051 | 0.1090 | 0.3371 | 0.6217 |
| Phosphatidylcholine | | | | | |
| (2.5% w/v) | | | | | |
| Chitosan (0.5% w/v)-2 | 0.0000 | 0.0008 | 0.0018 | 0.0711 | 0.3406 |
| Poly-GuD (0.56% w/v) | 0.0000 | 0.0008 | 0.0059 | 0.0180 | 0.0461 |
| pH = 6.4 | | | | | |
| Chitosan (0.5% w/v)-3 | 0.0000 | 0.0000 | 0.0042 | 0.0130 | 0.7870 |
| EDTA (0.5% w/v) | 0.0000 | 0.0000 | 0.0000 | 0.0123 | 0.5163 |
| Phorpol (6 × 10⁻⁶% w/v) | 0.0000 | 0.0000 | 0.0000 | 0.0000 | 0.0020 |
| Sodium Turocholate | 0.0000 | 0.4053 | 0.8205 | 1.6684 | 2.6868 |
| (2.0% w/v) | | | | | |
| Poly-GuD 0.56% (w/v) | 0.0000 | 0.0000 | 0.0054 | 0.1095 | 0.8920 |
| pH = 4.2 | | | | | |

TABLE 9
Influence of Pharmaceutical Formulations Comprising Interferon-
α-2b and Intranasal Delivery-Enhancing Agents on Permeation of
Interferon-α-2b through EpiAirway Cell Membrane as Measured by
ELISA Assay

| Pharmaceutical | % Permeated at Time-intervals (min) | | | | | |
|---|---|---|---|---|---|---|
| Formulations | 0 min | 15 min | 30 min | 60 min | 120 min | Fold Increase |
| Intron - A (IFN-α-2b) | 0 | 0.000 | 0.003 | 0.011 | 0.022 | 1 |
| F-2 | 0 | 0.064 | 0.434 | 1.443 | 3.013 | 135 |
| (IFN-α-2b, Chitosan, | | | | | | |
| MBCD, SDC) | | | | | | |
| F3 | 0 | 0.013 | 0.049 | 0.179 | 0.562 | 25 |
| (IFN-α-2b, Chitosan, | | | | | | |
| SDC), concentration (60 MIU/mL), | | | | | | |
| 230.4 ug/mL | | | | | | |
| F4 | 0 | 0.001 | 0.006 | 0.036 | 0.064 | 3 |
| (IFN-α-2b, Chitosan, | | | | | | |
| MBCD, EDTA) | | | | | | |
| F5 | 0 | 0.009 | 0.011 | 0.023 | 0.061 | 3 |
| (IFN-α-2b, Carbopol | | | | | | |
| 934P, Bacitracin, Oleic | | | | | | |
| Acid) | | | | | | |
| F6 | 0 | 0.000 | 0.001 | 0.010 | 0.059 | 3 |
| (IFN-α-2b, Carbopol | | | | | | |
| 934P, Bacitracin, EDTA) | | | | | | |
| F8 | 0 | 0.016 | 0.054 | 0.210 | 0.761 | 34 |
| (IFN-α-2b, Carbopol | | | | | | |
| 934, SDC) | | | | | | |
| F9 | 0 | 0.004 | 0.032 | 0.086 | 0.183 | 8 |
| (IFN-α-2b, Olic, Pectin)) | | | | | | |
| F10 | 0 | 0.025 | 0.123 | 0.324 | 0.559 | 25 |
| (IFN-α-2b, HPMC, STD- | | | | | | |
| Colate, Lisophosphatidyl | | | | | | |
| Choline) | | | | | | |
| F13 | 0 | 0.002 | 0.018 | 0.093 | 0.318 | 14 |
| (IFN-α-2b, HPMC, Poly- | | | | | | |
| L-Arginine, SNAP, | | | | | | |
| Sodium Caproic Acid) | | | | | | |
| F2-R | 0 | 0.0040 | 0.0408 | 0.1499 | 0.4275 | 37 |
| (IFN-α-2b, Poly (GuD), | | | | | | |
| Anti-Inflammatory | | | | | | |
| PEG-Intron A | 0 | 0.0 | 0.003 | 0.004 | 0.005 | 1 |
| (interferon-α-2b) | | | | | | |
| F14 | 0 | 0.02 | 0.08 | 0.15 | 0.28 | 56 |
| (PEG-Intron-A; IFN-α- | | | | | | |
| 2b, BAC, αPCD, MBCD, | | | | | | |
| EDTA) | | | | | | |

MTTAssay:

The MTT assays were performed using MTT-100, MatTek kits 300 mL of the MTT solution was added into each well. Tissue inserts were gently rinsed with clean PBS and placed in the MTT solution. The samples were incubated at 37° C. for 3 hours. After incubation the cell culture inserts were then immersed with 2.0 mL of the extractant solution per well to completely cover each insert. The extraction plate was covered and sealed to reduce evaporation. Extraction proceeds overnight at RT in the dark. After the extraction period was complete, the extractant solution was mixed and pipetted into a 96-well microtiter plate. Triplicates of each sample were loaded, as well as extractant blanks. The optical density of the samples was then measured at 550 nm on a plate reader (Molecular Devices).

The MTT assay on an exemplary formulation for enhanced mucosal delivery of IFN-α (e.g., Formulations F-2 and F-2-R) show results that indicate that there is minimal toxic effect of these exemplary embodiments on viability of the mucosal epithelial tissue. The results for intranasal delivery enhancing agent, IFN-α in combination with poly-GuD (0.56% w/v, pH=6.4), indicate that there is minimal toxic effect of this exemplary embodiment on viability of the mucosal epithelial tissue. Furthermore, exemplary formulations F-3, F-8, F-9, F-4, F-5, F-6 F-10, F-13 indicate that there is minimal toxic effect of this exemplary embodiment on viability of the mucosal epithelial tissue.

LDHAssay:

The LDH assay on exemplary formulations of the present invention for enhanced mucosal delivery of interferon-α are shown in Tables 10 and 11. The results show that there is minimal toxic effect of an exemplary embodiment, IFN-α in combination with chitosan (0.5% w/v) or poly-GuD (0.56% w/v, pH=6.4), on viability of the mucosal epithelial tissue See Table 10. The results show that there is minimal toxic effect of an exemplary embodiment, Formulations F-2 and F-2-R, on viability of the mucosal epithelial tissue. See Table 11. Exemplary Formulation F-14 showed no significant cytotoxicity by LDH assay or MTT assay See Table 11.

TABLE 10
Influence of Pharmaceutical Formulations Comprising Interferon-α-
2b and Intranasal Delivery-Enhancing Agents on the Viability of
EpiAirway cell membrane as shown by % Dead Cells (LDH Assay)

| Formulation with | % Dead Cells at Time Points (min) | | | | |
|---|---|---|---|---|---|
| Interferon-α-2b | 0 | 15 | 30 | 60 | 120 |
| Control (No Treatment) | 0.000 | 0.395 | 0.441 | 0.511 | 0.650 |
| Pluronic-127 (0.3% w/v) | 0 | 0.325 | 0.325 | 0.325 | 0.372 |
| Chitosan (0.5% w/v) | 0 | 0.627 | 0.720 | 1.602 | 3.506 |
| Alpha - Cyclodextrin | 0 | 2.206 | 2.415 | 2.508 | 2.624 |
| (5% w/v) | | | | | |
| Gamma Cyclodexrin | 0 | 0.720 | 0.789 | 0.859 | 0.975 |
| (1% w/v) | | | | | |
| N-Caproic Acid Sodium | 0 | 0.395 | 0.395 | 0.418 | 0.488 |
| (0.138% w/v) | | | | | |
| Palmitoyl-DL-Carnitine | 0 | 0.836 | 0.952 | 1.091 | 1.277 |
| (0.02% w/v) | | | | | |
| Didecanyol-1-Alpha- | 0 | 0.673 | 0.836 | 1.115 | 1.602 |
| Phosphatidylcholine | | | | | |
| (2.5% w/v) | | | | | |
| Chitosan (0.5% w/v)-2 | 0 | 0.093 | 0.116 | 0.557 | 1.579 |
| Poly-GuD 0.56% (w/v) | 0 | 0.046 | 0.046 | 0.046 | 0.093 |
| pH = 6.4 | | | | | |
| Chitosan (0.5% w/v)-3 | 0 | 0.139 | 0.255 | 1.045 | 2.670 |
| EDTA (0.5% w/v) | 0 | 0.209 | 0.302 | 0.673 | 1.672 |
| Phorpol (6 × 10⁻⁶% w/v) | 0 | 0.186 | 0.395 | 0.580 | 0.673 |
| Sodium Turocholate (2.0 w/v) | 0 | 1.207 | 2.020 | 4.203 | 7.105 |
| Poly-GuD 0.56% (w/v) | 0 | 0.232 | 0.441 | 1.393 | 4.226 |
| PH = 4.2 | | | | | |

TABLE 11
Influence of Pharmaceutical Formulations Comprising Interferon-α-
2b and Intranasal Delivery-Enhancing Agents on the Viability of
EpiAirway cell membrane as shown by % Dead Cells (LDH Assay)

| | % Dead Cells at Time-Intervals (min) | | | | |
|---|---|---|---|---|---|
| Formulation | 0 | 15 | 30 | 60 | 120 |
| Control (No Treatment) | 0 | 1.95 | 2.09 | 2.35 | 2.88 |
| Intron-A (IFN-α-2b) | 0 | 0.84 | 0.93 | 1.65 | 4.27 |
| F2 (IFN-α-2b, Chitosan, MBCD, | 0 | 1.56 | 3.90 | 5.76 | 8.10 |
| SDC) | | | | | |
| F2-R (IFN-α-2b, Poly(GuD), | 0 | 0.116 | 0.627 | 0.789 | 0.813 |
| Anti-Inflam.) | | | | | |
| F3 (IFN-α-2b, Chitosan, MB-CD, | 0 | 0.95 | 1.95 | 4.74 | 8.66 |
| EDTA) | | | | | |
| F4 (IFN-α-2b, Chitosan, SDC) | 0 | 2.25 | 2.76 | 3.07 | 3.34 |
| F5 (IFN-α-2b, Carbopol 934P, | 0 | 0.42 | 0.58 | 0.79 | 1.04 |
| Bacitracin, Oleic) | | | | | |
| F6 (IFN-α-2b, Carbopol 934P, | 0 | 0.63 | 0.86 | 1.00 | 1.18 |
| Bacitracin, EDTA) | | | | | |
| F8 (IFN-α-2b, Carbopol 934, | 0 | 1.09 | 3.60 | 10.77 | 23.71 |
| SDC) | | | | | |
| F9 (IFN-α-2b, Olic, Pectin)) | 0 | 0.56 | 1.11 | 2.04 | 3.09 |
| F13 (Interferon-α-2b) | 0 | 0.55 | 0.64 | 0.78 | 1.96 |
| F14 | 0 | 0.88 | 2.8 | 5.9 | 9.9 |
| (PEG-Intron-A; IFN-α-2b, BAC, | | | | | |
| αPCD, MBCD, EDTA) | | | | | |

### EXAMPLE 5

### Formulation F2-R of the Present Invention In Combination With Triamcinolone Acetonide Corticosteroid Improves Cell Viability

The present example provides an in vitro study to determine the permeability and reduction in epithelial mucosal inflammation of an intranasally administered interferon-α, for example, human interferon-α, in combination with a steroid composition, for example, triamcinolone acetonide, and further in combination with one or more intranasal delivery-enhancing agents. The study involves determination of epithelial cell permeability by TER assay and reduction in epithelial mucosal inflammation as measured by cell viability in an MTT assay by application of an embodiment comprising interferon-α and triamcinolone acetonide.

Formulation F2-R (Intron A® (Interferona-α2b), poly (GuD), acetic acid, benzalkonium chloride, sodium deoxycholate, methyl-β-cyclodextrin, EDTA, sodium hydroxide; see Table 4 above) is combined in a formulation with triamcinolone acetonide at a dosage of 0.5, 2.0, 5.0, or 50 μg. Normal dose of triamcinolone acetonide, (Nasacort®, Aventis Pharmaceuticals) for seasonal allergic rhinitis, is 55 μg per spray. Formulation F2-R in combination with triamcinolone acetonide corticosteroid improves cell viability as measured by the MTT assay, while maintaining epithelial cell permeability as measured by TER and ELISA assays.

According to the methods and formulations of the invention, measurement of permeability of Formulation F2-R in the presence or absence of triamcinolone acetonide is performed by transepithelial electrical resistance (TER) assays in an EpiAirway™ cell membrane. TER assays of Formulation F2-R plus triamcinolone acetonide at a concentration of 0.5, 2.0, 5.0, or 50 μg per spray indicate that interferon-α permeability did not decrease and was equal to permeability of Formulation F2-R alone. Formulation F2-R plus triamcinolone acetonide at a triamcinolone acetonide concentration between 0 and 50 μg per spray is typically, at least 10-fold greater than permeability of interferon-α in an IntronA® control.

According to the methods and formulations of the invention, measurement of permeability of Formulation F2-R in the presence or absence of triamcinolone acetonide is performed by ELISA assay in an EpiAirway™ cell membrane. Similar to the TER assay above, ELISA assay of Formulation F2-R plus triamcinolone acetonide at a concentration of 0.5, 2.0, 5.0, or 50 μg per spray indicate that interferon-α permeability did not decrease and was equal to permeability of Formulation F2-R alone. Formulation F2-R plus triamcinolone acetonide at a triamcinolone acetonide concentration between 0 and 50 μg per spray is typically greater than permeability of an IntronA® control (interferon-α).

According to the methods and formulations of the invention, MTT assay measured cell viability of Formulation F2-R in the presence or absence of triamcinolone acetonide. Typically, addition of triamcinolone acetonide (at a concentration of 0.5, 2.0, 5.0, or 50 μg per spray) to Formulation F2-R improves cell viability compared to Formulation F2-R in the absence of triamcinolone acetonide.

Addition of triamcinolone acetonide to Formulation F2-R increases cell viability and maintains epithelial permeability as measured by TER assay comparable to Formulation F2-R in the absence of triamcinolone acetonide.

Reduction in epithelial mucosal inflammation of an intranasally administered interferon-α is accomplished with an intranasal formulation of interferon-α in combination with one or more steroid or corticosteroid compound(s) typically high potency compounds or formulations, but also in certain cases medium potency, or low potency compounds or formulations. Overall potency (equivalent dosages) of high, medium, and low potency steroids are given. An intranasal formulation of interferon-α in combination with one or more steroid or corticosteroid compound(s) is useful for treatment of steroid myopathy due to chronic steroid use, for example, in treatment of viral infection, such as acute or chronic hepatitis B or hepatitis C, or tumor disease. Typically, an intranasal formulation of interferon-α in combination with a high potency steroid composition includes, but is not limited to, betamethasone (0.6 to 0.75 mg dosage), or dexamethasone (0.75 mg dosage). In an alternative formulation, an intranasal formulation of interferon-α in combination with a medium potency steroid composition includes, but is not limited to, methylprednisolone (4 mg dosage), triamcinolone (4 mg dosage), or prednisolone (5 mg dosage). In a further alternative formulation, an intranasal formulation of interferon-α in combination with a low potency steroid composition includes, but is not limited to hydrocortisone (20 mg dosage) or cortisone (25 mg dosage).

### EXAMPLE 6

### Bioavailability and Bioactivity of Three Different Doses of Nasal Interferon-α (IFN-α) Administered to Healthy Male Volunteers: Comparison with Subcutaneous Administration

STUDY SYNOPSIS.

The present example provides a non-blinded study to determine the uptake of intranasally administered interferon-α in combination with one or more intranasal delivery-enhancing agents into the blood serum in healthy male volunteers. The study involves administration of an intranasal effective amount of an exemplary formulation of the invention, Formulation F-2, as described above, to evaluate the absorption and tolerance of the interferon-α intranasal formulation by the subjects.

REFERENCE PRODUCT.

The nasal reference products were reconstituted using Intron A® (Interferon α-2b powder for injection at 50 MIU per vial; Schering Corp.; 50 MIU=192 μg of Interferon α-2b, recombinant) to provide an interferon-α-2b reference dose of 3 MIU/0.10 mL.

TEST FORMULATION (F-2) PRODUCT.

Test Products 1, 2 and 3 of Protocol 2, below, contain Formulation F-2 of the present invention (Intron A®, Interferon-α-2b, powder for injection at 50 MIU per vial; Schering Corp.; 50 MU=192 μg of recombinant Interferon α-2b, chitosan( CHITOCLEAR® 95% DAC from Primex, Inc.), acetic acid, benzalkonium chloride, sodium deoxycholate (from Sigma Aldrich), methyl-β-cyclodextrin (from Sigma Aldrich), EDTA at a pH of 5±0.2; see Table 4 in Example 3) to provide interferon-α-2b doses of 3 MIU/0.10 mL, 6 MIU/0.10 mL and 12 MIU/0.10 mL.

PROTOCOL.

Twelve healthy male subjects are enrolled in the step and each receives all 3 test product in a cross over fashion. The test products are administered in the following dose escalation manner:

Protocol 1:

Reference (Control/Intron A®) Dosage: Subcutaneous=5 MIU (Subjects 1-12)

Test Product (Formulation F-2) Dosage: Intranasal=5 MIU (One week wash out period; Subjects 1-12)

Test Product (Formulation F-2) Dosage: Intranasal=10 MIU (One week wash out period; Subjects 1-12)

The intranasal product formulation is manufactured under GMP conditions. Storage conditions is at 5° C.

Ten healthy male subjects plus 2 healthy male subjects from Protocol 1 were used in Protocol 2. Each receive all 3 test products. The 2 patients from Protocol 1 will serve as a comparison to the previous step.

The protocol involved three doses of the same test product in a dose escalation manner. This dose escalation manner is to ensure safety. Subjects were dosed in the following sequence:

Protocol 2:

Test Product 1 Dosage: Intranasal; Formulation F-2=3 MIU

Test Product 2 Dosage: Intranasal; Formulation F-2=6 MIU

Test Product 3 Dosage: Intranasal; Formulation F-2 =12 MIU

Reference (Control/Intron A®) Dosage: Subcutaneous;=3 MIU

Each subject received Formulation F-2 as an intranasal dose at 3 MIU, 6 MIU or 12 MIU dosage concentration. Each subject simultaneously received Intron A as a subcutaneous dose at 3 MIU.

The absorption and tolerance results of all products tested were tabulated and analyzed for Cₘₐₓ, Tₘₐₓ, and bioavailability (Area under concentration curve, AUC).

For nasal and injectable preparations in Protocols 1 and 2, 7 mL blood samples were drawn at 0 (prior to dose), 10, 20, 30, 45, 60, 75, 90, 120, 180, 240, 360, and 480 minutes post dosing into appropriate vacutainers.

The blood samples were centrifuged and the serum assayed for drug concentration using ELISA Method. The kit is as follows: Human Interferon Alpha for Human Serum Kit; Product Number 41110; Supplier: PBL Biomedical Laboratories, NJ.

TRIAL DESIGN:

This is a single dose, parallel group study to evaluate absorption, tolerance and pharmacodynamic parameters of Interferon-α-2b by two routes of administration: subcutaneous and intranasally. The study involves twelve healthy male subjects randomly assigned six per group (6 subjects subcutaneous and 6 intranasal). The objective of the study is to evaluate the absorption, tolerance and pharmacodynamic parameters of intranasal administration of Interferon-α-2b by formulations of the present invention versus subcutaneous administration of interferon-α-2b (IntronA®, Schering, Corp.). Each subject visits the clinical site ten times within a 6 month period. These visits consist of a screening visit, one dosing visit and eight safety monitoring visits.

SUBJECTS.

This study involves twelve healthy male subjects for the initial screening of a potential intranasal formulation.

TREATMENT PLAN, DOSAGE.

Before dosing, all subjects were given an orientation of the proper dosing technique and general conduct of the study. When receiving the intranasal dosage formulations, the subjects were seated and instructed to gently blow their nose before dosing. During dosing, the other nostril must be closed with the forefinger. They were also instructed to tilt their heads slightly back for dosing and to return their heads to an upright position while sniffing in gently immediately following dosing. Subjects must avoid additional sniffing and must remain in a seated position with head upright for 5 minutes after dosing. Subjects must inform the staff if they sneeze or if the product drips out of their nose.

The blood samples were collected in 7 mL vacutainers and centrifuged at room temperature for not less than 8 minutes at 1,500 rpm after at least 30 minutes of blood draw. At least 1.2 mL of serum was pipetted into the first of two polypropylene tubes, with the remainder pipetted into the second tube. Both tubes were frozen promptly and stored at −10° C. for no more than 30 days until shipment for analysis. When instructed by the study monitor, the first samples (containing at least 1.2 mL of serum) were placed in test tube racks packed on dry ice sufficient for 2 days and shipped (with a complete inventory of samples sent) for delivery via overnight mail to the analytical laboratory. The second sample was retained by the Investigator until the study monitor notifies him/her of the appropriate disposition.

Serum drug concentrations were measured using a suitable analytical method. The concentration at each sampling time and the appropriate pharmacokinetic parameters were reported.

MONITORING OF SUBJECTS.

Demographic data, subject initials, gender, age, race and statement of non-smoking status were recorded. A complete medical history and physical examination including electrocardiogram, vital signs, height and weight, and the following laboratory tests were conducted at screening and when the subject completes the study. Blood chemistry, hematology, urinalysis, drug screens are performed on each subject.

ABSORPTION DATA EVALUATION.

All absorption data was plotted for individual subjects as well as for the averaged data. The Cₘₐₓ, tₘₐₓ and the bioavailability (measured as area under the individual serum interferon-αconcentration vs. time curves, AUC) of the test products were evaluated. The goal was to compare the aforementioned pharmacokinetic parameters for formulations, Formulation F-2, as described above, with Intron A, interferon-α-2b, administered subcutaneously.

STATISTICS: Determination of AUC.

The areas under the individual serum interferon-αconcentration vs. time curves (AUC) were calculated according to the linear trapezoidal rule and with addition of the residual areas. A decrease of 23% or an increase of 30% between two dosages would be detected with a probability of 90% (type II error β=10%). The rate of absorption was estimated by comparison of the time (tₘₐₓ) to reach the maximum concentration (Cₘₐₓ). Both Cₘₐₓ and tₘₐₓ were analyzed using non-parametric methods. Comparisons of the pharmacokinetics of sc, iv and intranasal interferon-αadministrations were performed by analysis of variance (ANOVA). For pairwise comparisons a Bonferroni-Holmes sequential procedure was used to evaluate significance. The dose-response relationship between the three nasal doses was estimated by regression analysis. P<0.05 was considered significant. Results are given as mean values +/−SEM.

RESULTS.

Due to its unique characteristics, the intranasal administration of pharmaceutical formulations of the present invention comprising interferon-αand one or more intranasal delivery-enhancing agents offers many advantages in terms of providing absorption of macromolecular drugs which are either not absorbed or variably absorbed after oral administration or absorbed more slowly following intramuscular or subcutaneous injection.

TABLE 12
Pharmacokinetic and Pharmacodynamic Parameters Measured as
Plasma concentrations of Interferon-α-2b in human subjects
expressed as Cₘₐₓ, tₘₐₓ, and AUC (0-3 h and 0-4 h),
comparing intranasal (IN) administration of interferon-α to
subcutaneous (SC) injection of interferon-α

| Interferon-α Formulation | | | AUC |
|---|---|---|---|
| and Dose | Cₘₐₓ (IU/mL) | tₘₐₓ (hr) | ng/mL · hr |
| 3 MIU, SC, | | | |
| (Control/Intron A ®) | | | |
| 0 to 3 hour | 4.0 | 18 to 36 | 1150 |
| 0 to 4 hour | 6.8 | | |
| 6 MIU, IN, | | | |
| F-2 formulation | | | |
| 0 to 6 hour | 2.6 | 1.4 | 610 |
| 12 MIU, IN, | | | |
| F-2 formulation | | | |
| 0 to 6 hour | 3.8 | 1.08 | 1,239 |

Table 12 provides pharmacokinetic data for intranasal delivery of interferon-α-2b in a pharmaceutical formulation of the present invention (e.g., Formulation F-2) compared to subcutaneous delivery of Intron A® control(interferon-α-2b; Schering Corporation).

The results exemplify bioavailability of interferon-α achieved by the methods and formulations herein, e.g., as measured by area under the concentration curve (AUC) in blood serum, CNS, CSF or in another selected physiological compartment or target tissue. Bioavailability of interferon-α will be, for example, AUC_{0-6 hr} for interferon-α of approximately 400 ng·hr /mL of blood plasma or CSF, AUC_{0-6 hr} for interferon-α of approximately 700 ng·hr /mL of blood plasma or CSF, or AUC_{0-6 hr} for interferon-α up to approximately 1300 ng·hr/mL of blood plasma or CSF.

The results exemplify bioavailability of interferon-α achieved by the methods and formulations herein. For example, maximum concentration of interferon-α in the blood serum (Cₘₐₓ) at 3 hours post dosing was 4.0 IU/mL for subcutaneous delivery of Intron A (at 3 MIU dose) compared to 3.8 IU/mL for intranasal delivery of Formulation F-2 (at 12 MIU dose). Similar Cₘₐₓ values were obtained for the intranasal formulation of the present invention and the subcutaneous formulation of the marketed product.

For example, time to maximum serum concentration of interferon-α in the blood serum (tₘₐₓ) is 15- to 30-fold faster for intranasal delivery of the formulation of the present invention (e.g., Formulation F-2) compared to subcutaneous delivery of Intron A (interferon-α-2b). tₘₐₓ for Formulation F-2 is 1.3 hours compared to a tₘₐₓ of 18 to 36 hours for subcutaneous administration of Intron A.

Elimination rate of interferon-α from the intranasal delivery site of Formulation F-2 of the present invention is consistent with data for the elimination rate of interferon-α from the subcutaneous delivery site of Intron A (interferon-α-2b). Formulation F2 elimination constant is 3.9 hours for 3 MIU or 12 MIU dose by intranasal administration. Intron A elimination constant is 3 to 12 hours for 5 MIU dose by subcutaneous injection.

The results indicate that significant plasma levels (Cₘₐₓ) of interferon-α are achieved following intranasal administration of a pharmaceutical formulation of interferon-α in combination with one or more intranasal delivery-enhancing agents of the present invention. The time to maximum serum concentration (tₘₐₓ) by intranasal delivery is accelerated 15- to 30 fold to achieve similar blood plasma levels when compared to subcutaneous injection. The rate of delivery of interferon-α by intranasal administration of pharmaceutical formulations of the present invention (as measured by Cₘₐₓ and tₘₐₓ) is significantly increased when compared to subcutaneous injection of interferon-α.

The potential to deliver and maintain consistent therapeutic blood levels of interferon-α by pharmaceutical formulations of the present invention provide a distinct advantage over existing formulations for subcutaneous administration. A distinct advantage exists for maintaining consistent therapeutic blood levels of interferon-α by repeated intranasal administration within a 1 to 2 hour time frame in which maximum concentration in the blood serum is achieved, as compared to subcutaneous administration which requires 4 hours or longer to reach maximum concentration in the blood serum.

Although the foregoing invention has been described in detail by way of example for purposes of clarity of understanding, it will be apparent to the artisan that certain changes and modifications are comprehended by the disclosure and may be practiced without undue experimentation within the scope of the appended claims, which are presented by way of illustration not limitation.

What is claimed is:

1. A stable pharmaceutical composition comprising one or more interferon-α compound(s) formulated for mucosal delivery to a mammalian subject wherein said composition following mucosal administration to said subject yields enhanced mucosal delivery of said one or more interferon-α compound(s), and wherein said composition is effective to alleviate one or more symptom(s) of said viral infection or tumor disease in said subject without unacceptable adverse side effects.

2. The pharmaceutical composition of claim 1, further comprising one or more mucosal delivery-enhancing agent(s).

3. The pharmaceutical composition of claim 2, wherein said composition is formulated for nasal mucosal delivery to a mammalian subject.

4. The pharmaceutical composition of claim 4, wherein said composition is formulated as an intranasal spray or powder.

5. The pharmaceutical composition of claim 1, wherein said composition is effective following mucosal administration to alleviate one or more symptom(s) of chronic or acute hepatitis B infection or chronic or acute hepatitis C infection in said subject without unacceptable adverse side effects

6. The pharmaceutical composition of claim 1, wherein said composition is effective following mucosal administration to alleviate one or more symptom(s) of condyloma acuminata, hairy cell leukemia, Kaposi's sarcoma, chronic myelogenous leukemia (CML), B and T cell lymphoma, midgut carcinoid tumors, metastasizing renal cell carcinoma, malignant melanoma, follicular lymphoma, or myeloma in said subject without unacceptable adverse side effects.

7. The pharmaceutical composition of claim 1, further comprising a plurality of different interferon-α compounds.

8. The pharmaceutical composition of claim 1, wherein said composition following mucosal administration to said subject yields enhanced mucosal delivery of said one or more interferon-α compound(s) characterized by: (i) a peak concentration (Cₘₐₓ) of said interferon-α compound(s) in a CNS tissue or fluid or in a blood plasma of said subject that is 15% or greater as compared to a peak concentration of said interferon-α compounds in CNS or blood plasma following subcutaneous injection of an equivalent concentration or dose of said interferon-α compound(s) to said subject; (ii) an area under concentration curve (AUC) of said interferon-α compound(s) in a central nervous system (CNS) tissue or fluid or in a blood plasma of the subject that is 25% or greater compared to an AUC of interferon-α in CNS or blood plasma following subcutaneous injection of an equivalent concentration or dose of said interferon-α compound(s) to said subject; or (iii) a time to maximal concentration (tₘₐₓ) of said interferon-α in a central nervous system (CNS) tissue or fluid or in a blood plasma of the subject between about 0.1 to 1.0 hours.

9. The pharmaceutical composition of claim 1, wherein said composition following mucosal administration to said subject yields a peak concentration (Cₘₐₓ) of said interferon-α compound(s) in said CNS tissue or fluid or in a blood plasma of said subject that is 25% or greater as compared to a peak concentration of said interferon-α compound(s) in said CNS tissue or fluid or blood plasma following subcutaneous injection of an equivalent concentration or dose of said interferon-α compound(s) to said subject.

10. The pharmaceutical composition of claim 9, wherein said composition following mucosal administration to said subject yields a peak concentration (Cₘₐₓ) of said interferon-α compound(s) in said CNS tissue or fluid or in a blood plasma of said subject that is 50% or greater as compared to a peak concentration of said interferon-α compound(s) in said CNS or blood plasma following subcutaneous injection of an equivalent concentration or dose of said interferon-α compound(s) to said subject.

11. The pharmaceutical composition of claim 1, wherein said composition following mucosal administration to said subject yields an area under concentration curve (AUC) of said interferon-α compound(s) in said CNS tissue or fluid or in a blood plasma of the subject that is 25% or greater compared to an AUC of said interferon-α compound(s) in said CNS or blood plasma following subcutaneous injection of an equivalent concentration or dose of said interferon-α compound(s) to said subject.

12. The pharmaceutical composition of claim I 1, wherein said composition following mucosal administration to said subject yields an area under concentration curve (AUC) of said interferon-α compound(s) in said CNS tissue or fluid or in a blood plasma of the subject that is 50% or greater compared to an AUC of said interferon-α compound(s) in said CNS or blood plasma following subcutaneous injection of an equivalent concentration or dose of said interferon-α compound(s) to said subject.

13. The pharmaceutical composition of claim 1, wherein said composition following mucosal administration to said subject yields a time to maximal plasma concentration (tₘₐₓ) of said interferon-α compound(s) in said CNS tissue or fluid or in a blood plasma of the subject between about 0.1 to 1.0 hours.

14. The pharmaceutical composition of claim 13, wherein said composition following mucosal administration to said subject yields a time to maximal plasma concentration (tₘₐₓ) of said interferon-α compound(s) in said CNS tissue or fluid or in a blood plasma of the subject between about 0.2 to 0.5 hours.

15. The pharmaceutical composition of claim 1, wherein said composition following mucosal administration to said subject yields a peak concentration of said interferon-α compound(s) in said CNS tissue or fluid of the subject that is 10% or greater compared to a peak concentration of said interferon-α compound(s) in a blood plasma of the subject.

16. The pharmaceutical composition of claim 15, wherein said composition following mucosal administration to said subject yields a peak concentration of said interferon-α compound(s) in said CNS tissue or fluid of the subject that is 20% or greater compared to a peak concentration of said interferon-α compound(s) in a blood plasma of the subject.

17. The pharmaceutical composition of claim 16, wherein said composition following mucosal administration to said subject yields a peak concentration of said interferon-α compound(s) in said CNS tissue or fluid of the subject that is 40% or greater compared to a peak concentration of said interferon-α compound(s) in a blood plasma of the subject.

18. The pharmaceutical composition of claim 1, wherein said interferon-α compound(s) formulated for intranasal delivery to said subject in combination with said one or more intranasal delivery-enhancing agent(s) is effective following intranasal administration to alleviate one or more symptom(s) of viral infection or tumor in said subject without unacceptable adverse side effects.

19. The pharmaceutical composition of claim 2, wherein said mucosal delivery-enhancing agent(s) is/are selected from:
(a) an aggregation inhibitory agent;
(b) a charge-modifying agent;
(c) a pH control agent;
(d) a degradative enzyme inhibitory agent;
(e) a mucolytic or mucus clearing agent;
(f) a ciliostatic agent;
(g) a membrane penetration-enhancing agent selected from (i) a surfactant, (ii) a bile salt, (ii) a phospholipid additive, mixed micelle, liposome, or carrier, (iii) an alcohol, (iv) an enamine, (v) an NO donor compound, (vi) a long-chain amphipathic molecule (vii) a small hydrophobic penetration enhancer; (viii) sodium or a salicylic acid derivative; (ix) a glycerol ester of acetoacetic acid (x) a cyclodextrin or beta-cyclodextrin derivative, (xi) a medium-chain fatty acid, (xii) a chelating agent, (xiii) an amino acid or salt thereof, (xiv) an N-acetylamino acid or salt thereof, (xv) an enzyme degradative to a selected membrane component, (ix) an inhibitor of fatty acid synthesis, or (x) an inhibitor of cholesterol synthesis; or (xi) any combination of the membrane penetration enhancing agents recited in (i)-(x);
(h) a modulatory agent of epithelial junction physiology;
(i) a vasodilator agent;
(j) a selective transport-enhancing agent; and
(k) a stabilizing delivery vehicle, carrier, support or complex-forming species with which the interferon-α is effectively combined, associated, contained, encapsulated or bound resulting in stabilization of the interferon-α for enhanced nasal mucosal delivery, wherein the formulation of said interferon-α with said one or more intranasal delivery-enhancing agents provides for increased bioavailability of the interferon-α in a blood plasma of said subject.

20. The pharmaceutical composition of claim 19, further comprising a plurality of mucosal delivery-enhancing agents.

21. The pharmaceutical composition of claim 19, comprising one or more intranasal delivery-enhancing agents.

22. The pharmaceutical composition of claim 21, further comprising a plurality of intranasal delivery-enhancing agents.

23. The pharmaceutical composition of claim 2, wherein said mucosal delivery-enhancing agent(s) is/are selected from the group consisting of citric acid, sodium citrate, propylene glycol, glycerin, L-ascorbic acid, sodium metabisulfite, EDTA disodium, benzalkonium chloride, sodium hydroxide and mixtures thereof.

24. The pharmaceutical composition of claim 1, further comprising one or more sustained release-enhancing agent(s).

25. The pharmaceutical composition of claim 24, wherein the sustained release-enhancing agent is polyethylene glycol (PEG) in combination with interferon-α.

26. The pharmaceutical composition of claim 1, wherein the interferon-α is human interferon-α or a biologically active analog, fragment, or derivative thereof.

27. The pharmaceutical composition of claim 1, wherein said interferon-α is formulated in an effective dosage unit of between about 30 and 250 μg.

28. The pharmaceutical composition of claim 1, further comprising one or more steroid or corticosteroid compound(s), wherein said composition is effective following mucosal administration to alleviate one or more symptom(s) of inflammation, nasal irritation, rhinitis, or allergy without unacceptable adverse side effects.

29. The pharmaceutical formulation of claim 1, which is pH adjusted to between about pH 3.0-6.0.

30. The pharmaceutical formulation of claim 1, which is pH adjusted to between about pH 3.0-5.0.

31. The pharmaceutical formulation of claim I, which is pH adjusted to between about pH 4.0-5.0.

32. The pharmaceutical formulation of claim 1, which is pH adjusted to about pH 4.0-4.5.

33. The pharmaceutical formulation of claim 2, wherein said mucosal delivery-enhancing agent is a permeabilizing peptide that reversibly enhances mucosal epithelial paracellular transport by modulating epithelial junctional structure and/or physiology in a mammalian subject, wherein said peptide effectively inhibits homotypic binding of an epithelial membrane adhesive protein selected from a junctional adhesion molecule (JAM), occludin, or claudin.

34. A method for treating or preventing a viral or tumor disease or condition in a mammalian subject amenable to treatment by therapeutic administration of a interferon-α compound comprising administering to a mucosal surface of said subject a pharmaceutical composition comprising an effective amount of one or more interferon-α compound(s) formulated for mucosal delivery in combination with one or more mucosal delivery-enhancing agent(s) in an effective dosage regimen to alleviate one or more symptom(s) of said viral infection or tumor in said subject without unacceptable adverse side effects.

35. The method of claim 34, wherein said interferon-α compound(s) is/are formulated for intranasal delivery to said subject in combination with one or more intranasal delivery-enhancing agent(s), and wherein said method employs an intranasal effective dosage regimen to alleviate one or more symptom(s) of said viral infection or tumor in said subject without unacceptable adverse side effects.

36. The method of claim 35, wherein said interferon-α compound(s) is/are provided in a multiple dosage unit kit or container for repeated self-dosing by said subject.

37. The method of claim 35, wherein said interferon-α compound(s) is/are repeatedly administered through an intranasal effective dosage regimen that involves multiple administrations of said interferon-α compound(s) to said subject during a daily or weekly schedule to maintain a therapeutically effective baseline level of interferon-α during an extended dosing period.

38. The method of claim 37, wherein said interferon-α compound(s) is/are self-administered by said subject in a nasal formulation between two and six times daily to maintain a therapeutically effective baseline level of interferon-α during an 8 hour to 24 hour extended dosing period.

39. The method of claim 34, which yields a peak concentration (Cₘₐₓ) of said interferon-α in a blood plasma or cerebral spinal fluid (CNS) of said subject following mucosal administration that is 25% or greater as compared to a peak concentration of interferon-α in blood plasma or CNS following subcutaneous injection of an equivalent concentration or dose of interferon-a to said subject.

40. The method of claim 39, which yields a peak concentration (Cₘₐₓ) of said interferon-α in a blood plasma or cerebral spinal fluid (CNS) of said subject following mucosal administration that is 50% or greater as compared to a peak concentration of interferon-α in blood plasma or CNS following subcutaneous injection of an equivalent concentration or dose of interferon-α to said subject.

41. The method of claim 34, which yields an area under concentration curve (AUC) of said interferon-α in a blood plasma or cerebral spinal fluid (CNS) of the subject following mucosal administration that is 25% or greater compared to an AUC of interferon-α in blood plasma or CNS following subcutaneous injection of an equivalent concentration or dose of interferon-α to said subject.

42. The method of claim 41, which yields an area under concentration curve (AUC) of said interferon-α in a blood plasma or cerebral spinal fluid (CNS) of the subject following mucosal administration that is 50% or greater compared to an AUC of interferon-α in blood plasma or CNS following subcutaneous injection of an equivalent concentration or dose of interferon-α to said subject.

43. The method of claim 34, which yields a time to maximal plasma concentration (tₘₐₓ) of said interferon-α in a blood plasma or cerebral spinal fluid (CNS) of the subject following mucosal administration of between about 0.1 to 1.0 hours.

44. The method of claim 43, which yields a time to maximal plasma concentration (tₘₐₓ) of said interferon-α in a blood plasma or cerebral spinal fluid (CNS) of the subject following mucosal administration of between 0.2 to 0.5 hours.

45. The method of claim 34, which yields a peak concentration of said interferon-α in a central nervous system (CNS) tissue or fluid of the subject following mucosal administration that is 10% or greater compared to a peak concentration of said interferon-α in a blood plasma of the subject.

46. The method of claim 45, which yields a peak concentration of said interferon-α in a central nervous system (CNS) tissue or fluid of the subject following mucosal administration that is 20% or greater compared to a peak concentration of said interferon-α in a blood plasma of the subject.

47. The method of claim 45, which yields a peak concentration of said interferon-α in a central nervous system (CNS) tissue or fluid of the subject following mucosal administration that is 40% or greater compared to a peak concentration of said interferon-α in a blood plasma of the subject.

48. The method of claim 34, wherein said mucosal delivery-enhancing agent(s) is/are selected from:
(a) an aggregation inhibitory agent;
(b) a charge-modifying agent;
(c) a pH control agent;
(d) a degradative enzyme inhibitory agent;
(e) a mucolytic or mucus clearing agent;
(f) a ciliostatic agent;
(g) a membrane penetration-enhancing agent selected from (i) a surfactant, (ii) a bile salt, (ii) a phospholipid additive, mixed micelle, liposome, or carrier, (iii) an alcohol, (iv) an enamine, (v) an NO donor compound, (vi) a long-chain amphipathic molecule (vii) a small hydrophobic penetration enhancer; (viii) sodium or a salicylic acid derivative; (ix) a glycerol ester of acetoacetic acid (x) a cyclodextrin or beta-cyclodextrin derivative, (xi) a medium-chain fatty acid, (xii) a chelating agent, (xiii) an amino acid or salt thereof, (xiv) an N-acetylamino acid or salt thereof, (xv) an enzyme degradative to a selected membrane component, (ix) an inhibitor of fatty acid synthesis, or (x) an inhibitor of cholesterol synthesis; or (xi) any combination of the membrane penetration enhancing agents recited in (i)-(x);
(h) a modulatory agent of epithelial junction physiology;
(i) a vasodilator agent;
(j) a selective transport-enhancing agent; and
(k) a stabilizing delivery vehicle, carrier, support or complex-forming species with which the interferon-α is effectively combined, associated, contained, encapsulated or bound resulting in stabilization of the interferon-α for enhanced nasal mucosal delivery, wherein the formulation of said interferon-α with said one or more intranasal delivery-enhancing agents provides for increased bioavailability of the interferon-α in a blood plasma of said subject.

49. The method of claim 48, wherein said pharmaceutical composition further comprises a plurality of mucosal delivery-enhancing agents.

50. The method of claim 34, wherein said pharmaceutical composition comprises one or more intranasal delivery-enhancing agents.

51. The method of claim 50, wherein said pharmaceutical composition comprises a plurality of intranasal delivery-enhancing agents.

52. The method of claim 34, wherein said mucosal delivery-enhancing agent(s) is/are selected from the group consisting of citric acid, sodium citrate, propylene glycol, glycerin, L-ascorbic acid, sodium metabisulfite, EDTA disodium, benzalkonium chloride, sodium hydroxide and mixtures thereof.

53. The method of claim 34, wherein said pharmaceutical composition further comprises one or more sustained release-enhancing agent(s).

54. The method of claim 53, wherein the sustained release-enhancing agent is polyethylene glycol (PEG).

55. The method of claim 34, wherein the interferon-α is human interferon-α or a biologically active analog, fragment, or derivative thereof.

56. The method of claim 34, wherein said interferon-α is formulated in an effective dosage unit of between about 30 and 250 μg.

57. The method of claim 34, which is effective to alleviate one or more symptom(s) of chronic or acute hepatitis B infection or chronic or acute hepatitis C infection in said subject without unacceptable adverse side effects.

58. The method of claim 34, which is effective to alleviate one or more symptom(s) of condyloma acuminata, hairy cell leukemia, Kaposi's sarcoma, chronic myelogenous leukemia (CML), B and T cell lymphoma, midgut carcinoid tumors, metastasizing renal cell carcinoma, malignant melanoma, follicular lymphoma, and myeloma in said subject without unacceptable adverse side effects.

59. The method of claim 34, wherein said pharmaceutical composition comprises a plurality of different interferon-α compounds.

60. A pharmaceutical composition suitable for intranasal administration of interferon alpha comprised water, interferon alpha, chitosan, and methyl-beta-cyclodextrin.

61. The pharmaceutical composition of claim 60 wherein the composition has a pH of about 4-6.

62. The pharmaceutical composition of claim 61 wherein the pH is about 5.0.
